SEQUENCE LISTING (71 sequences)

SEQ ID NO: 1 - protein, 457 aa, Thermococcus hydrothermalis
  SIGNAL 1..25
MARKVLVALLVFLVVLSVSAVPAKAETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 2 - protein, 460 aa, Pyrococcus furiosus
MNIKKLTPLLTLLLFFIVLASPVSAAKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 3 - protein, 549 aa, Geobacillus stearothermophilus
  SIGNAL 1..34
MLTFHRIIRKGWMFLLAFLLTALLFCPTGQPAKAAAPFNGTMMQYFEWYLPDDGTLWTKVANEANNLSSLGITALWLPPAYKGTSRSDVGYGVYDLYDLGEFNQKGAVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKSWGKWYVNTTNIDGFRLDAVKHIKFSFFPDWLSDVRSQTGKPLFTVGEYWSYDINKLHNYIMKTNGTMSLFDAPLHNKFYTASKSGGTFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIAWSITTRPWTDEFVRWTEPRLVAWP

SEQ ID NO: 4 - protein, 515 aa, Geobacillus stearothermophilus
AAPFNGTMMQYFEWYLPDDGTLWTKVANEANNLSSLGITALWLPPAYKGTSRSDVGYGVYDLYDLGEFNQKGAVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKSWGKWYVNTTNIDGFRLDAVKHIKFSFFPDWLSDVRSQTGKPLFTVGEYWSYDINKLHNYIMKTNGTMSLFDAPLHNKFYTASKSGGTFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIAWSITTRPWTDEFVRWTEPRLVAWP

SEQ ID NO: 5 - protein, 548 aa, Geobacillus stearothermophilus
  SIGNAL 1..34
MLTFHRIIRKGWMFLLAFLLTASLFCPTGRHAKAAAPFNGTMMQYFEWYLPDDGTLWTKVANEANNLSSLGITALSLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDGLKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTNPAKRCSHGRPWFKPLAYAFILTRQEGYPCVFYGDYYGIPQYNIPSLKSKIDPLLIARRDYAYGTQHDYLDHSDIIGWTREGVTEKPGSGLAALITDGAGRSKWMYVGKQHAGKVFYDLTGNRSDTVTINSDGWGEFKVNGGSVSVWVPRKTTVSTIARPITTRPWTGEFVRWHEPRLVAWP

SEQ ID NO: 6 - protein, 549 aa, Geobacillus stearothermophilus
  SIGNAL 1..34
MLTFHRIIRKGWMFLLAFLLTVSLFCPTGQPAKAAAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 7 - protein, 476 aa, Thermococcus gammatolerans
MGVVPALSQGGMEMRRYTRVLILLMALFLLAGLYYPSASAAKYSELEQGGVIMQAFYWDVPAGGIWWDTIRQKIPEWYDAGISAIWIPPASKGMGGAYSMGYDPYDYFDLGEFYQKGTVETRFGSKEELVNMISTAHRYGIKVIADIVINHRAGGDLEWNPYVGDYTWTDFSQVASGKYKAHYMDFHPNNYSTSDEGTFGGFPDIDHLVPFNKYWLWASDESYAAYLRSIGVDAWRFDYVKGYGAWVVKDWLSWWGGWAVGEYWDTDVNALLNWAYDSGAKVFDFPLYYKMDEAFDNKNIPALVYAIQNGGTVVSRDPFKAVTFVANHDTNIIWNKYPAYAFILTYEGQPVIFYRDYEEWLNKDKLNNLIWIHEHLAGGSTKILYYDDDELIFMREGYGDRPGLITYINLGSGWAERWVNVGSKFAGYTIHEYTGNLGGWVDRYVYYNGWVKLTAPPHDPANGYYGYSVWSYAGVG

SEQ ID NO: 8 - protein, 457 aa, Thermococcus thioreducens
MARKVTVALLVLLVVLSLSAVPAKAETLENGGVIMQAFYWDVPMGGIWWDTIAQKIPDWASAGISAIWIPPASKGMSGGYSMGYDPYDYFDLGEYYQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASNESYAAYLRSIGIDAWRFDYVKGYAPWVVKDWLNWWGGWAVGEYWDTNVDALLNWAYASGAKVFDFPLYYKMDEAFDNNNIPALVDALRYGQTVVSRDPFKAVTFVANHDTDIIWNKYPAYAFILTYEGQPMIFYRDYEEWLNKDRLKNLIWIHDHLAGGSTDIVYYDSDELIFVRNGYGSKPGLITYINLGSSKAGRWVYVPKFAGSCIHEYTGNLGGWVDKWVDSSGWVYLEAPAHDPANGQYGYSVWSYCGVG

SEQ ID NO: 9 - protein, 461 aa, Thermococcus eurythermalis
MKPAKLLVFVLVVSILAGLYAQPAGAAKYLELEEGGVIMQAFYWDVPSGGIWWDTIRQKIPEWYDAGISAIWIPPASKGMGGAYSMGYDPYDFFDLGEYDQKGTVETRFGSKQELVNMINTAHAYGIKVIADIVINHRAGGDLEWNPFVNDYTWTDFSKVASGKYTANYLDFHPNEVKCCDEGTFGGFPDIAHEKSWDQYWLWASNESYAAYLRSIGVDAWRFDYVKGYGAWVVKDWLDWWGGWAVGEYWDTNVDALLNWAYSSDAKVFDFPLYYKMDAAFDNKNIPALVEALKNGGTVVSRDPFKAVTFVANHDTDIIWNKYPAYAFILTYEGQPTIFYRDYEEWLNKDRLKNLIWIHDHLAGGSTDIVYYDNDELIFVRNGYGDKPGLITYINLGSSKAGRWVYVPKFAGACIHEYTGNLGGWVDKWVDSSGWVYLEAPAHDPANGYYGYSVWSYCGVG

SEQ ID NO: 10 - protein, 435 aa, Thermococcus gammatolerans
KYSELEQGGVIMQAFYWDVPAGGIWWDTIRQKIPEWYDAGISAIWIPPASKGMGGAYSMGYDPYDYFDLGEFYQKGTVETRFGSKEELVNMISTAHRYGIKVIADIVINHRAGGDLEWNPYVGDYTWTDFSQVASGKYKAHYMDFHPNNYSTSDEGTFGGFPDIDHLVPFNKYWLWASDESYAAYLRSIGVDAWRFDYVKGYGAWVVKDWLSWWGGWAVGEYWDTDVNALLNWAYDSGAKVFDFPLYYKMDEAFDNKNIPALVYAIQNGGTVVSRDPFKAVTFVANHDTNIIWNKYPAYAFILTYEGQPVIFYRDYEEWLNKDKLNNLIWIHEHLAGGSTKILYYDDDELIFMREGYGDRPGLITYINLGSGWAERWVNVGSKFAGYTIHEYTGNLGGWVDRYVYYNGWVKLTAPPHDPANGYYGYSVWSYAGVG

SEQ ID NO: 11 - protein, 432 aa, Thermococcus thioreducens
ETLENGGVIMQAFYWDVPMGGIWWDTIAQKIPDWASAGISAIWIPPASKGMSGGYSMGYDPYDYFDLGEYYQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASNESYAAYLRSIGIDAWRFDYVKGYAPWVVKDWLNWWGGWAVGEYWDTNVDALLNWAYASGAKVFDFPLYYKMDEAFDNNNIPALVDALRYGQTVVSRDPFKAVTFVANHDTDIIWNKYPAYAFILTYEGQPMIFYRDYEEWLNKDRLKNLIWIHDHLAGGSTDIVYYDSDELIFVRNGYGSKPGLITYINLGSSKAGRWVYVPKFAGSCIHEYTGNLGGWVDKWVDSSGWVYLEAPAHDPANGQYGYSVWSYCGVG

SEQ ID NO: 12 - protein, 440 aa, Thermococcus eurythermalis
QPAGAAKYLELEEGGVIMQAFYWDVPSGGIWWDTIRQKIPEWYDAGISAIWIPPASKGMGGAYSMGYDPYDFFDLGEYDQKGTVETRFGSKQELVNMINTAHAYGIKVIADIVINHRAGGDLEWNPFVNDYTWTDFSKVASGKYTANYLDFHPNEVKCCDEGTFGGFPDIAHEKSWDQYWLWASNESYAAYLRSIGVDAWRFDYVKGYGAWVVKDWLDWWGGWAVGEYWDTNVDALLNWAYSSDAKVFDFPLYYKMDAAFDNKNIPALVEALKNGGTVVSRDPFKAVTFVANHDTDIIWNKYPAYAFILTYEGQPTIFYRDYEEWLNKDRLKNLIWIHDHLAGGSTDIVYYDNDELIFVRNGYGDKPGLITYINLGSSKAGRWVYVPKFAGACIHEYTGNLGGWVDKWVDSSGWVYLEAPAHDPANGYYGYSVWSYCGVG

SEQ ID NO: 13 - protein, 432 aa, Thermococcus hydrothermalis
ETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 14 - protein, 434 aa, Pyrococcus furiosus
KYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 15 - protein, 436 aa, Thermococcus gammatolerans
MKYSELEQGGVIMQAFYWDVPAGGIWWDTIRQKIPEWYDAGISAIWIPPASKGMGGAYSMGYDPYDYFDLGEFYQKGTVETRFGSKEELVNMISTAHRYGIKVIADIVINHRAGGDLEWNPYVGDYTWTDFSQVASGKYKAHYMDFHPNNYSTSDEGTFGGFPDIDHLVPFNKYWLWASDESYAAYLRSIGVDAWRFDYVKGYGAWVVKDWLSWWGGWAVGEYWDTDVNALLNWAYDSGAKVFDFPLYYKMDEAFDNKNIPALVYAIQNGGTVVSRDPFKAVTFVANHDTNIIWNKYPAYAFILTYEGQPVIFYRDYEEWLNKDKLNNLIWIHEHLAGGSTKILYYDDDELIFMREGYGDRPGLITYINLGSGWAERWVNVGSKFAGYTIHEYTGNLGGWVDRYVYYNGWVKLTAPPHDPANGYYGYSVWSYAGVG

SEQ ID NO: 16 - protein, 436 aa, Pyrococcus furiosus
MSKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 17 - protein, 433 aa, Thermococcus thioreducens
METLENGGVIMQAFYWDVPMGGIWWDTIAQKIPDWASAGISAIWIPPASKGMSGGYSMGYDPYDYFDLGEYYQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASNESYAAYLRSIGIDAWRFDYVKGYAPWVVKDWLNWWGGWAVGEYWDTNVDALLNWAYASGAKVFDFPLYYKMDEAFDNNNIPALVDALRYGQTVVSRDPFKAVTFVANHDTDIIWNKYPAYAFILTYEGQPMIFYRDYEEWLNKDRLKNLIWIHDHLAGGSTDIVYYDSDELIFVRNGYGSKPGLITYINLGSSKAGRWVYVPKFAGSCIHEYTGNLGGWVDKWVDSSGWVYLEAPAHDPANGQYGYSVWSYCGVG

SEQ ID NO: 18 - protein, 441 aa, Thermococcus eurythermalis
MQPAGAAKYLELEEGGVIMQAFYWDVPSGGIWWDTIRQKIPEWYDAGISAIWIPPASKGMGGAYSMGYDPYDFFDLGEYDQKGTVETRFGSKQELVNMINTAHAYGIKVIADIVINHRAGGDLEWNPFVNDYTWTDFSKVASGKYTANYLDFHPNEVKCCDEGTFGGFPDIAHEKSWDQYWLWASNESYAAYLRSIGVDAWRFDYVKGYGAWVVKDWLDWWGGWAVGEYWDTNVDALLNWAYSSDAKVFDFPLYYKMDAAFDNKNIPALVEALKNGGTVVSRDPFKAVTFVANHDTDIIWNKYPAYAFILTYEGQPTIFYRDYEEWLNKDRLKNLIWIHDHLAGGSTDIVYYDNDELIFVRNGYGDKPGLITYINLGSSKAGRWVYVPKFAGACIHEYTGNLGGWVDKWVDSSGWVYLEAPAHDPANGYYGYSVWSYCGVG

SEQ ID NO: 19 - protein, 433 aa, Thermococcus hydrothermalis
METLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 20 - protein, 436 aa, Pyrococcus furiosus
MAKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 21 - protein, 435 aa, Pyrococcus furiosus
MKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 22 - protein, 435 aa, Pyrococcus furiosus
MKYSELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 23 - protein, 435 aa, Thermococcus hydrothermalis
MKYETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 24 - protein, 434 aa, Thermococcus hydrothermalis
MSETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 25 - protein, 597 aa, synthetic construct
  SIGNAL 1..19
MRFPSIFTAVLFAASSALAETLENGGVIMQAFYWDVPGGGIWNDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVGAEAAAKALEAEAAAKEAAAKEAAAKEAAAKAAANVTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 26 - protein, 486 aa, synthetic construct
  SIGNAL 1..19
MRFPSIFTAVLFAASSALAKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVGEAAKEAAKEAAKAANARAIPGALGLAGAVMMLL

SEQ ID NO: 27 - protein, 517 aa, Geobacillus stearothermophilus
MSAAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 28 - protein, 534 aa, Geobacillus stearothermophilus
MSAAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWPKRPAAATKKAGQAKKKK

SEQ ID NO: 29 - protein, 772 aa, synthetic construct
  SIGNAL 1..19
MRFPSIFTAVLFAASSALAAAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWPEAAKEAAKEAAKALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAM

SEQ ID NO: 30 - protein, 451 aa, Thermococcus hydrothermalis
MSETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVGKRPAAATKKAGQAKKKK

SEQ ID NO: 31 - protein, 19 aa, Saccharomyces cerevisiae
MRFPSIFTAVLFAASSALA

SEQ ID NO: 32 - protein, 12 aa, synthetic construct
EAAKEAAKEAAK

SEQ ID NO: 33 - protein, 34 aa, synthetic construct
AEAAAKALEAEAAAKEAAAKEAAAKEAAAKAAAN

SEQ ID NO: 34 - protein, 226 aa, Saccharomyces cerevisiae
ALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAM

SEQ ID NO: 35 - protein, 112 aa, Saccharomyces cerevisiae
VTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 36 - protein, 21 aa, Saccharomyces cerevisiae
AANARAIPGALGLAGAVMMLL

SEQ ID NO: 37 - protein, 17 aa, Xenopus laevis
KRPAAATKKAGQAKKKK

SEQ ID NO: 38 - protein, 514 aa, Geobacillus stearothermophilus
AAPFNGTMMQYFEWYLPDDGTLWTKVANEANNLSSLGITALSLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDGLKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTNPAKRCSHGRPWFKPLAYAFILTRQEGYPCVFYGDYYGIPQYNIPSLKSKIDPLLIARRDYAYGTQHDYLDHSDIIGWTREGVTEKPGSGLAALITDGAGRSKWMYVGKQHAGKVFYDLTGNRSDTVTINSDGWGEFKVNGGSVSVWVPRKTTVSTIARPITTRPWTGEFVRWHEPRLVAWP

SEQ ID NO: 39 - protein, 515 aa, Geobacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 40 - protein, 512 aa, Bacillus licheniformis
MKQQKRLYARLLTLLFALIFLLPHSAAAAANLNGTLMQYFEWYTPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEYLIKAWTHFHFPGRGSTYSDFKWHWYHFDGTDWDESRKLNRIYKFQGKAWDWEVSSENGNYDYLMYADIDYDHPDVVAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 41 - protein, 512 aa, Bacillus licheniformis; identical to SEQ ID NO: 40
  SIGNAL 1..29

SEQ ID NO: 42 - protein, 436 aa, synthetic construct
MAKYSELEKGGVIMQAFYWDVPSGGIWWDTIRQKIPEWYDAGISAIWIPPASKGMGGAYSMGYDPYDFFDLGEYDQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASQESYAAYLRSIGIDAWRFDYVKGYAPWVVKDWLNWWGGWAVGEYWDTNVDAVLNWAYSSGAKVFDFALYYKMDEAFDNKNIPALVSALQNGQTVVSRDPFKAVTFVANHDTDIIWNKYPAYAFILTYEGQPTIFYRDYEEWLNKDKLKNLIWIHENLAGGSTDIVYYDNDELIFVRNGYGDKPGLITYINLGSSKAGRWVYVPKFAGACIHEYTGNLGGWVDKYVYSSGWVYLEAPAYDPANGQYGYSVWSYCGVG

SEQ ID NO: 43 - protein, 513 aa, synthetic construct
AAPFNGTMMQYFEWYLPDDGTLWTKVANEANNLSSLGITALWLPPAYKGTSRSDVGYGVYDLYDLGEFNQKGAVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTENGNYDYLMYADLDMDHPEVVTELKSWGKWYVNTTNIDGFRLDAVKHIKFSFFPDWLSDVRSQTGKPLFTVGEYWSYDINKLHNYIMKTNGTMSLFDAPLHNKFYTASKSGGTFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIAWSITTRPWTDEFVRWTEPRLVAWP

SEQ ID NO: 44 - protein, 513 aa, synthetic construct
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 45 - protein, 513 aa, synthetic construct
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTEFGNYDYLMYADLDMDHPEVVTELKNWGKWYVNTTNIDGFRLDAVKHIKFAFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 46 - protein, 513 aa, synthetic construct; identical to SEQ ID NO: 45

SEQ ID NO: 47 - protein, 513 aa, synthetic construct
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTEFGNYDYLMYADLDMDHPEVVTELKNWGKWYVNTTNIDGFRLDAVKHIKFAFFRDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 48 - protein, 513 aa, synthetic construct
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWYWYHFDGVDWDESRKLSRIYKFRGKTWDWEVDTEFGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 49 - protein, 513 aa, synthetic construct
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWYWYHFDGVDWDESRKLKRIYKFRGKTWDWEVDTEFGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 50 - protein, 513 aa, synthetic construct
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWYWYHFDGVDWDESRKLSRIYKFRGKTWDWEVDPEFGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 51 - protein, 513 aa, synthetic construct
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWYWYHFDGVDWDESRKLSRIYKFRGKTWDWEVDTEFGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 52 - protein, 534 aa, synthetic construct; identical to SEQ ID NO: 28
  INIT_MET 1

SEQ ID NO: 53 - protein, 533 aa, synthetic construct
SAAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWPKRPAAATKKAGQAKKKK

SEQ ID NO: 54 - protein, 597 aa, synthetic construct; identical to SEQ ID NO: 25
  SIGNAL 1..19

SEQ ID NO: 55 - protein, 578 aa, synthetic construct
ETLENGGVIMQAFYWDVPGGGIWNDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVGAEAAAKALEAEAAAKEAAAKEAAAKEAAAKAAANVTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 56 - protein, 451 aa, synthetic construct
  INIT_MET 1
MSETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSQIHEYTGNLGGWIDKWVDSSGRVYLEAPAHDPANGQYGYSVWSYVGVGKRPAAATKKAGQAKKKK

SEQ ID NO: 57 - protein, 433 aa, synthetic construct
SETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSQIHEYTGNLGGWIDKWVDSSGRVYLEAPAHDPANGQYGYSVWSYVGVG

SEQ ID NO: 58 - protein, 542 aa, synthetic construct
  SIGNAL 1..92
MRQVWFSWIVGLFLCFFNVSSAAPVNTTTEDETAQIPAEAVIGYSDLEGDFDVAVLPFSNSTNNGLLFINTTIASIAAKEEGVSLEKREAEASETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSNTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSQIHEYTGNLGGWIDKWVDSSGRVYLEAPAHDPANGQYGYSVWSYVGVGKRPAAATKKAGQAKKKK

SEQ ID NO: 59 - protein, 433 aa, synthetic construct
SETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSNTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSQIHEYTGNLGGWIDKWVDSSGRVYLEAPAHDPANGQYGYSVWSYVGVG

SEQ ID NO: 60 - protein, 451 aa, synthetic construct; identical to SEQ ID NO: 30

SEQ ID NO: 61 - protein, 534 aa, synthetic construct
  SIGNAL 1..19
MRFPSIFTAVLFAASSALAAAPFNGTMMQYFEWYLPDDGTLWTKVANEANNLSSLGITALWLPPAYKGTSRSDVGYGVYDLYDLGEFNQKGAVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKSWGKWYVNTTNIDGFRLDAVKHIKFSFFPDWLSDVRSQTGKPLFTVGEYWSYDINKLHNYIMKTNGTMSLFDAPLHNKFYTASKSGGTFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIAWSITTRPWTDEFVRWTEPRLVAWP

SEQ ID NO: 62 - protein, 534 aa, synthetic construct
  SIGNAL 1..19
MRFPSIFTAVLFAASSALAAAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 63 - protein, 532 aa, synthetic construct
  SIGNAL 1..19
MRFPSIFTAVLFAASSALAAAPFNGTMMQYFEWYLPDDGTLWTKVANEANNLSSLGITALWLPPAYKGTSRSDVGYGVYDLYDLGEFNQKGAVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTENGNYDYLMYADLDMDHPEVVTELKSWGKWYVNTTNIDGFRLDAVKHIKFSFFPDWLSDVRSQTGKPLFTVGEYWSYDINKLHNYIMKTNGTMSLFDAPLHNKFYTASKSGGTFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIAWSITTRPWTDEFVRWTEPRLVAWP

SEQ ID NO: 64 - protein, 532 aa, synthetic construct
  SIGNAL 1..19
MRFPSIFTAVLFAASSALAAAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 65 - protein, 435 aa, Thermococcus hydrothermalis; identical to SEQ ID NO: 23

SEQ ID NO: 66 - protein, 456 aa, synthetic construct
  SIGNAL 1..19
MRFPSIFTAVLFAASSALAAPAKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 67 - protein, 433 aa, Thermococcus hydrothermalis
SETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 68 - protein, 544 aa, synthetic construct
  SIGNAL 1..30
MRSKKLWISLLFALTLIFTMAFSNMSAQAAAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 69 - protein, 544 aa, synthetic construct
  SIGNAL 1..30
MRSKKLWISLLFALTLIFTMAFSNMSAQAAAPFNGTMMQYFEWYLPDDGTLWTKVANEANNLSSLGITALWLPPAYKGTSRSDVGYGVYDLYDLGEFNQKGAVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKSWGKWYVNTTNIDGFRLDAVKHIKFSFFPDWLSDVRSQTGKPLFTVGEYWSYDINKLHNYIMKTNGTMSLFDAPLHNKFYTASKSGGTFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIAWSITTRPWTDEFVRWTEPRLVAWP

SEQ ID NO: 70 - protein, 434 aa, synthetic construct
MSETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSNTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSQIHEYTGNLGGWIDKWVDSSGRVYLEAPAHDPANGQYGYSVWSYVGVG

SEQ ID NO: 71 - protein, 514 aa, synthetic construct
MAAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

(19) United States
(12) Patent Application Publication
(10) Pub. No.: US 2025/0179458 A1
(43) Pub. Date: Jun. 5, 2025

(54) BACTERIAL AND ARCHAEAL ALPHA-AMYLASES

(71) Applicant: Danstar Ferment AG, Zug (CH)

(72) Inventors: Charles F. Rice, Lebanon, NH (US); Christopher J. Freeman, Lebanon, NH (US); Hannah Green, Lebanon, NH (US); Aaron Argyros, Lebanan, NH (US); Alexandra-Elena Panaitiu, Lebanon, NH (US); Ryan Skinner, Lebanon, NH (US); Anne K. Woodbrey, Lebanon, NH (US)

(21) Appl. No.: 18/844,795

(22) PCT Filed: Mar. 9, 2023
(86) PCT No.: PCT/IB2023/052263

§ 371 Date: Sep. 6, 2024

Related U.S. Application Data
(60) Provisional application No. 63/318,294, filed on Mar. 9, 2022.

(51) Int. Cl. C12N 9/28; C12N 1/16

(52) U.S. Cl. CPC C12N 9/2417; C12N 1/165; C07K 2319/00; C12Y 302/01001

(57) ABSTRACT

The present disclosure concerns an enzyme combination comprising at least one archaeal alpha-amylase and at least one bacterial alpha-amylase that can be used for the hydrolysis of a starchy biomass as well as the production of a fermentation product. The present disclosure also concerns variant polypeptides having alpha-amylase activity exhibiting higher enzymatic activity, lower dependence on the presence of a metallic ion, higher thermostability and/or higher resistance to chelation.

### CROSS-REFERENCE TO RELATED APPLICATION(S) AND DOCUMENT(S)

This application claims priority from U.S. provisional patent application 63/318,294 filed on Mar. 9, 2022 and herewith incorporated in its entirety. This application also comprises a sequence listing in electronic form which is also incorporated in its entirety.

### TECHNOLOGICAL FIELD

The present disclosure concerns alpha-amylases (from bacterial or archaeal origin) for hydrolyzing starch, recombinant microbial host cell making them as well as processes using them.

### BACKGROUND

In the biofuel industry, α-amylases are used in the liquefaction of starch-containing material to reduce viscosity (to facilitate downstream processing) and initiate hydrolysis of starch. The liquefaction process relies on a combination of high temperatures to gelatinize the starch and the exogenous addition of enzymes (which are generally thermostable) to hydrolyze starch molecules into shorter dextrins. The liquefied mash can then cooled and inoculated with a fermenting yeast optionally with the exogenous addition of purified enzymes such as, for example, glucoamylases which will further break down the dextrin into utilizable glucose molecules.

It would be highly desirable to be provided an enzyme preparation having improved α-amylase activity as well as enzymes having improved biological properties for the liquefaction of starch.

### BRIEF SUMMARY

The present disclosure concerns α-amylases which can be used to liquefy starch prior to the saccharification/fermentation processes. The present disclosure concerns an enzyme combination comprising at least two distinct α-amylases which can be used to liquefy starch prior to the saccharification/fermentation processes. The enzyme combination comprises at least one α-amylase from an archaeal origin and at least one α-amylase from a bacterial origin. The present disclosure also concerns bacterial/archaeal α-amylase variants having improved properties such as lower dependence on the presence of a metallic ion, improved thermostability and/or improved resistance towards chelation.

According to a first aspect, the present disclosure provides an enzyme combination comprising at least one archaeal alpha-amylase and at least one bacterial alpha-amylase. In an embodiment, the at least one archaeal alpha-amylase comprises a polypeptide derived from Thermococcus sp., such as, for example, from Thermococcus hydrothermalis. In some further embodiments, the at least one archaeal alpha-amylase comprises a polypeptide having the amino acid sequence of SEQ ID NO: 1, 13, 19, 23, 24, 25, 30, 54, 55, 56, 57, 58, 59, 60, 65, 67, or 70, or a variant of the polypeptide having the amino acid sequence of SEQ ID NO: 1, 13, 19, 23, 24, 25, 30, 54, 55, 56, 57, 58, 59, 60, 65, 67, or 70 exhibiting alpha-amylase activity. In an embodiment, the at least one archaeal alpha-amylase is the variant of the amino acid sequence of SEQ ID NO: 13, has at least 70% identity and less than 100% identity to the amino acid sequence of SEQ ID NO: 13, and is less dependent on the presence of a metallic ion, more thermostable and/or more resistant to chelation than the polypeptide having the amino acid sequence of SEQ ID NO: 13. For example the variant can have, at a position corresponding position 123 of the amino acid sequence of SEQ ID NO: 13, which is different from a tyrosine residue. In such embodiment, the variant can have, at the position corresponding position 123 of the amino acid sequence of SEQ ID NO: 13, an asparagine residue. In another example, the variant can have, at a position corresponding position 385 of the amino acid sequence of SEQ ID NO: 13, an amino acid residue which is different from a cysteine residue. In such embodiment, the variant can have, at the position corresponding position 385 of the amino acid sequence of SEQ ID NO: 13, a glutamine residue. In a further example, the variant can have, at a position corresponding position 429 of the amino acid sequence of SEQ ID NO: 13, an amino acid residue which is different from a cysteine residue. In such embodiment, the variant can have, at the position corresponding position 429 of the amino acid sequence of SEQ ID NO: 13, a valine residue. In an embodiment, the at least one archaeal alpha-amylase comprises a polypeptide derived from Pyrococcus sp., such as, for example, from the at least one archaeal alpha-amylase comprises a polypeptide derived from Pyrococcus furiosus. In a further embodiment, the at least one archaeal alpha-amylase comprises a polypeptide having the amino acid sequence of SEQ ID NO: 2, 14, 16, 20, 21, 22, 26, or 66, or a variant of the polypeptide having the amino acid sequence of SEQ ID NO: 2, 14, 16, 20, 21, 22, 26, or 66 exhibiting alpha-amylase activity. In still another embodiment, the enzyme combination comprises at least two archaeal alpha-amylases. In an embodiment, the at least one bacterial alpha-amylase comprises a polypeptide derived from Geobacillus sp., such as, for example, from Geobacillus stearothermophilus. In a further embodiment, the at least one bacterial alpha-amylase comprises a polypeptide having the amino acid sequence of any one of SEQ ID NO: 3, 4, 5, 6, 27, 28, 29, 38, 39, 43, 44, 45, 46, 47, 48, 49, 50, 51, 52, 53, 61, 62, 63, 64, 68, 69, or 71, or a variant of the polypeptide having the amino acid sequence of any one of 3, 4, 5, 6, 27, 28, 29, 38, 39, 43, 44, 45, 46, 47, 48, 49, 50, 51, 52, 53, 61, 62, 63, 64, 68, 69, or 71 exhibiting alpha-amylase activity. In an embodiment, the at least one bacterial alpha-amylase is the variant of the amino acid sequence of SEQ ID NO: 39, has at least 70% identity and less than 100% identity with the amino acid sequence of SEQ ID NO: 39, and is less dependent on the presence of a metallic ion, more thermostable and/or more resistant to chelation than the polypeptide having the amino acid sequence of SEQ ID NO: 39. In an embodiment, the variant has a deletion at a position corresponding to position 181 and/or position 182 of SEQ ID NO: 39. In still another embodiment, the variant has at least one of the following substitution: at a position corresponding to position 157 of SEQ ID NO: 39, an amino acid residue different than an arginine residue; at a position corresponding to position 173 of SEQ ID NO: 39, an amino acid residue different than a serine residue; at a position corresponding to position 184 of SEQ ID NO: 39, an amino acid residue different than an alanine residue; at a position corresponding to position 191 of SEQ ID NO: 39, an amino acid residue different than a threonine residue; at a position corresponding to position 193 of SEQ ID NO: 39, an amino acid residue different than an asparagine residue; at a position corresponding to position 242 of SEQ ID NO: 39, an amino acid residue different than a serine residue; at a position corresponding to position 245 of SEQ ID NO: 39, an amino acid residue different than a proline residue; or at a position corresponding to position 281 of SEQ ID NO: 39, an amino acid residue different than an aspartic acid residue. In an example, the variant has at least one: at a position corresponding to position 157 of SEQ ID NO: 39, a tyrosine residue; at a position corresponding to position 173 of SEQ ID NO: 39, a lysine residue at a position corresponding to position 184 of SEQ ID NO: 39, a threonine residue; at a position corresponding to position 191 of SEQ ID NO: 39, a proline residue; at a position corresponding to position 193 of SEQ ID NO: 39, a phenylalanine residue; at a position corresponding to position 242 of SEQ ID NO: 39, an alanine residue; at a position corresponding to position 245 of SEQ ID NO: 39, an arginine residue; or at a position corresponding to position 281 of SEQ ID NO: 39, an asparagine residue. In another embodiment, the enzyme combination further comprises a component of an inactivated microbe. In still another embodiment, the at least one archaeal alpha-amylase and/or the at least one bacterial alpha-amylase is provided in a substantially purified form.

According to a second aspect, the present disclosure provides a recombinant microbial host cell capable of expressing the enzyme combination defined herein. In an embodiment, the recombinant microbial host cell is a yeast host cell. In another embodiment, the recombinant microbial host cell is from the genus Saccharomyces sp., and, in additional embodiments, from the species Saccharomyces cerevisiae. In yet another embodiment, the recombinant microbial host cell of claim is from the genus Komagataella sp. and, in additional embodiments, from the species Komagataella phaffii. In an embodiment, the recombinant microbial host cell is a bacterial host cell. In yet another embodiment, the recombinant microbial host is from the genus Bacillus sp. and, in additional embodiments, from the species Bacillus subtilis.

According to a third aspect, the present disclosure provides an inactivated microbial product comprising the enzyme combination defined herein and a component of the recombinant microbial host cell defined herein.

According to a fourth aspect, the present disclosure provides a population of recombinant microbial host cells comprising a first subpopulation of recombinant microbial host cells capable of expressing the at least one archaeal alpha-amylase defined herein and a second subpopulation of recombinant microbial host cells capable of expressing the at least one bacterial alpha-amylase defined herein. In an embodiment, the first and/or the second subpopulation of recombinant microbial host cells comprises recombinant yeast host cells. In yet another embodiment, the first and/or the second subpopulation of recombinant microbial host cells comprises cells from the genus Saccharomyces sp., and, in a further embodiment. cells from the species Saccharomyces cerevisiae. In yet another embodiment, the first and/or the second subpopulation of recombinant microbial host cells comprises cells from the genus Komagataella sp. and, in further embodiments, from the species Komagataella phaffii. In an embodiment, the first and/or the second subpopulation of recombinant microbial host cells comprises recombinant bacterial host cells. In yet another embodiment, the first and/or the second subpopulation of recombinant microbial host cells comprises cells from the genus Bacillus sp. and, in further embodiments, from the species Bacillus subtilis.

According to a fifth embodiment, the present disclosure provides an inactivated microbial host cell product comprising the enzyme combination defined herein and a component of the first and/or second subpopulation of recombinant microbial host cells defined herein.

According to a sixth embodiment, the present disclosure provides a kit for the liquefaction of a biomass. The kit comprises the at least one archaeal alpha-amylase as described herein and the at least one bacterial alpha-amylase described herein. In an embodiment, the at least one archaeal alpha-amylase is provided: in a substantially purified form; by the recombinant microbial host cell described herein; by the inactivated microbial product described herein; and/or by the first subpopulation of recombinant microbial host cells described herein. In another embodiment, the at least one bacterial alpha-amylase is provided: in a substantially purified form; by the recombinant microbial host cell described herein; by the inactivated microbial product described herein; and/or by the second subpopulation of recombinant microbial host cells described herein.

According to a seventh aspect, the present disclosure provides an hydrolyzed liquefaction medium comprising the enzyme combination defined herein, the recombinant microbial host cell defined herein, the inactivated microbial product defined herein and/or the population of recombinant microbial host cells defined herein.

According to an eighth aspect, the present disclosure provides a process for making an hydrolyzed liquefaction medium. The process comprises (i) contacting an untreated liquefaction medium with the at least one archaeal alpha-amylase defined herein and the at least at least one bacterial alpha-amylase defined herein and (ii) hydrolyzing the untreated liquefaction medium to generate the hydrolyzed liquefaction medium. In an embodiment, step (i) comprises contacting the untreated liquefaction medium with the enzyme combination defined herein, the recombinant microbial host cell defined herein, the inactivated microbial product defined herein, the population of recombinant microbial host cells defined herein, and/or the kit described herein. In another embodiment, the process further comprises heating the untreated liquefaction medium at a liquefaction temperature and for a liquefaction time period to generate the hydrolyzed liquefaction medium. In yet another embodiment, the liquefaction temperature is at least 50° C. In yet a further embodiment, the liquefaction time period is at least 60 minutes. In an embodiment, the untreated liquefaction medium comprises corn. In still another embodiment, the hydrolyzed liquefaction medium is a gelatinized corn mash. In some embodiments, the process can be used for increasing the dextrose equivalent and/or decreasing the viscosity of the hydrolyzed liquefaction medium when compared to a control hydrolyzed liquefaction medium obtained with only one of an archaeal alpha-amylase or a bacterial alpha-amylase.

According to a ninth aspect, the present disclosure provides a process for making a fermented product. The process comprises contacting the hydrolyzed liquefaction medium defined herein or obtainable or obtained by the process defined herein with a fermenting yeast under a condition to allow the conversion of the hydrolyzed liquefaction medium into a fermentation product. In an embodiment, the fermentation product is an alcohol, such as, for example, ethanol. In still another embodiment, the hydrolyzed liquefaction medium is a gelatinized corn mash. In some embodiments, the process is for improving the yield of a fermentation (which can, in some embodiments, be normalized to fermentation solids), when compared to a control process contacting a control liquefaction medium obtained with only one of a control archaeal alpha-amylase or a control bacterial alpha-amylase.

According to a tenth aspect, the present disclosure provides a variant polypeptide having alpha-amylase activity, wherein the variant polypeptide has at least 70% identity and less than 100% identity to the amino acid sequence of SEQ ID NO: 13, and is less dependent of the presence of a metallic ion, more thermostable and/or less dependent on the presence of a metallic ion than the polypeptide consisting of the amino acid sequence of SEQ ID NO: 13. In an embodiment, the variant polypeptide has one or more amino acid residue substitution. For example, the variant polypeptide can have, at a position corresponding position 123 of the amino acid sequence of SEQ ID NO: 13, an amino acid residue which is different from a tyrosine residue. In such embodiment, the variant polypeptide can have, at the position corresponding position 123 of the amino acid sequence of SEQ ID NO: 13, an asparagine residue. In still another embodiment, the variant polypeptide can have, at a position corresponding position 385 of the amino acid sequence of SEQ ID NO: 13, an amino acid residue which is different from a cysteine residue. In such embodiment, the variant polypeptide can have, at the position corresponding position 385 of the amino acid sequence of SEQ ID NO: 13, a glutamine residue. In yet another example, the variant polypeptide can have, at a position corresponding position 429 of the amino acid sequence of SEQ ID NO: 13, an amino acid residue which is different from a cysteine residue. In such embodiment, the variant polypeptide can have, at the position corresponding position 429 of the amino acid sequence of SEQ ID NO: 13, a valine residue. In an embodiment, the present disclosure provides a recombinant microbial host cell capable of expressing the variant polypeptide described herein. In an embodiment, the recombinant microbial host cell comprises a recombinant yeast host cell. In yet another embodiment, the recombinant microbial host cell comprises a cell from the genus Saccharomyces sp., and, in a further embodiment, from the species Saccharomyces cerevisiae. In yet another embodiment, the recombinant microbial host cell comprises a cell from the genus Komagataella sp. and, in further embodiments, from the species Komagataella phaffii. In an embodiment, the recombinant microbial host cell comprises a recombinant bacterial host cells. In yet another embodiment, the recombinant microbial host cell comprises a cell from the genus Bacillus sp. and, in further embodiments, from the species Bacillus subtilis. In some embodiments, the present disclosure provides an inactivated microbial product comprising the variant polypeptide defined herein (optionally in combination with at least one bacterial alpha-amylase) and a component of the recombinant microbial host cell defined herein. In additional embodiments, the present disclosure provides an hydrolyzed liquefaction medium comprising the variant polypeptide defined herein (optionally in combination with at least one bacterial alpha-amylase), the recombinant microbial host cell defined herein, and/or the inactivated microbial product defined. In still yet further embodiments, the present disclosure provides a process for making an hydrolyzed liquefaction medium. The process comprises (i) contacting an untreated liquefaction medium with the variant polypeptide defined (optionally in combination with at least one bacterial alpha-amylase) herein and (ii) hydrolyzing the untreated liquefaction medium to generate the hydrolyzed liquefaction medium. In an embodiment, step (i) comprises contacting the untreated liquefaction medium with the substantially purified variant polypeptide defined herein, the recombinant microbial host cell defined herein, the inactivated microbial product defined herein. In another embodiment, the process further comprises heating the untreated liquefaction medium at a liquefaction temperature and for a liquefaction time period to generate the hydrolyzed liquefaction medium. In yet another embodiment, the liquefaction temperature is at least 50° C. In yet a further embodiment, the liquefaction time period is at least 60 minutes. In an embodiment, the untreated liquefaction medium comprises corn. In still another embodiment, the hydrolyzed liquefaction medium is a gelatinized corn mash. In some embodiments, the process can be used for increasing the dextrose equivalent and/or decreasing the viscosity of the hydrolyzed liquefaction medium when compared to a control hydrolyzed liquefaction medium obtained with the archaeal alpha-amylase consisting of the amino acid sequence of SEQ ID NO: 13. In yet additional embodiments, the present disclosure provides a process for making a fermented product. The process comprises contacting the hydrolyzed liquefaction medium defined herein or obtainable or obtained by the process defined herein with a fermenting yeast under a condition to allow the conversion of the hydrolyzed liquefaction medium into a fermentation product. In an embodiment, the fermentation product is an alcohol, such as, for example, ethanol. In still another embodiment, the hydrolyzed liquefaction medium is a gelatinized corn mash. In some embodiments, the process is for improving the yield of a fermentation (which can, in some embodiments, be normalized to the fermentation solids), when compared to a control process contacting a control liquefaction medium obtained with the archaeal alpha-amylase consisting of the amino acid sequence of SEQ ID NO: 13.

According to an eleventh aspect, the present disclosure provides a variant polypeptide having alpha-amylase activity, wherein the variant polypeptide has at least 70% identity and less than 100% identity with the amino acid sequence of SEQ ID NO: 39, and is less dependent on the presence of a metallic ion, more thermostable and/or more resistant to chelation than the polypeptide consisting of the amino acid sequence of SEQ ID NO: 39. In an embodiment, the variant polypeptide can have one or more amino acid residue deletion. In such embodiment, the variant polypeptide can have a deletion at a position corresponding to position 181 and/or position 182 of SEQ ID NO: 39. Alternatively or in combination, the variant polypeptide can have one or more amino acid residue substitution. For example, the variant polypeptide can have at least one of: at a position corresponding to position 157 of SEQ ID NO: 39, an amino acid residue different than an arginine residue; at a position corresponding to position 173 of SEQ ID NO: 39, an amino acid residue different than a serine residue; at a position corresponding to position 184 of SEQ ID NO: 39, an amino acid residue different than an alanine residue; at a position corresponding to position 191 of SEQ ID NO: 39, an amino acid residue different than an threonine residue; at a position corresponding to position 193 of SEQ ID NO: 39, an amino acid residue different than a asparagine residue; at a position corresponding to position 242 of SEQ ID NO: 39, an amino acid residue different than a serine residue; at a position corresponding to position 245 of SEQ ID NO: 39, an amino acid residue different than a proline residue; or at a position corresponding to position 281 of SEQ ID NO: 39, an amino acid residue different than an aspartic acid residue. In such embodiment, the variant polypeptide can have at least one of: at a position corresponding to position 157 of SEQ ID NO: 39, a tyrosine residue; at a position corresponding to position 173 of SEQ ID NO: 39, a lysine residue; at a position corresponding to position 184 of SEQ ID NO: 39, a threonine residue; at a position corresponding to position 191 of SEQ ID NO: 39, a proline residue; at a position corresponding to position 193 of SEQ ID NO: 39, a phenylalanine residue; at a position corresponding to position 242 of SEQ ID NO: 39, an alanine residue; at a position corresponding to position 245 of SEQ ID NO: 39, an arginine residue; or at a position corresponding to position 281 of SEQ ID NO: 39, an asparagine residue. In an embodiment, the present disclosure provides a recombinant microbial host cell capable of expressing the variant polypeptide described herein. In an embodiment, the recombinant microbial host cell comprises a recombinant yeast host cell. In yet another embodiment, the recombinant microbial host cell comprises a cell from the genus Saccharomyces sp., and, in a further embodiment, from the species Saccharomyces cerevisiae. In yet another embodiment, the recombinant microbial host cell comprises a cell from the genus Komagataella sp. and, in further embodiments, from the species Komagataella phaffii. In an embodiment, the recombinant microbial host cell comprises a recombinant bacterial host cells. In yet another embodiment, the recombinant microbial host cell comprises a cell from the genus Bacillus sp. and, in further embodiments, from the species Bacillus subtilis. In some embodiments, the present disclosure provides an inactivated microbial product comprising the variant polypeptide defined herein (optionally in combination with at least one archaeal alpha-amylase) and a component of the recombinant microbial host cell defined herein. In additional embodiments, the present disclosure provides an hydrolyzed liquefaction medium comprising the variant polypeptide defined herein (optionally in combination with at least one archaeal alpha-amylase), the recombinant microbial host cell defined herein, and/or the inactivated microbial product defined. In still yet further embodiments, the present disclosure provides a process for making an hydrolyzed liquefaction medium. The process comprises (i) contacting an untreated liquefaction medium with the variant polypeptide defined (optionally in combination with at least one archaeal alpha-amylase) herein and (ii) hydrolyzing the untreated liquefaction medium to generate the hydrolyzed liquefaction medium. In an embodiment, step (i) comprises contacting the untreated liquefaction medium with the substantially purified variant polypeptide defined herein, the recombinant microbial host cell defined herein, the inactivated microbial product defined herein. In another embodiment, the process further comprises heating the untreated liquefaction medium at a liquefaction temperature and for a liquefaction time period to generate the hydrolyzed liquefaction medium. In yet another embodiment, the liquefaction temperature is at least 50° C. In yet a further embodiment, the liquefaction time period is at least 60 minutes. In an embodiment, the untreated liquefaction medium comprises corn. In still another embodiment, the hydrolyzed liquefaction medium is a gelatinized corn mash. In some embodiments, the process can be used for increasing the dextrose equivalent and/or decreasing the viscosity of the hydrolyzed liquefaction medium when compared to a control hydrolyzed liquefaction medium obtained with the bacterial alpha-amylase consisting of the amino acid sequence of SEQ ID NO: 39. In yet additional embodiments, the present disclosure provides a process for making a fermented product. The process comprises contacting the hydrolyzed liquefaction medium defined herein or obtainable or obtained by the process defined herein with a fermenting yeast under a condition to allow the conversion of the hydrolyzed liquefaction medium into a fermentation product. In an embodiment, the fermentation product is an alcohol, such as, for example, ethanol. In still another embodiment, the hydrolyzed liquefaction medium is a gelatinized corn mash. In some embodiments, the process is for improving the yield of a fermentation (which can, in some embodiments, be normalized to fermentation solids), when compared to a control process contacting a control liquefaction medium obtained with the bacterial alpha-amylase consisting of the amino acid sequence of SEQ ID NO: 39.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 provides the size profile of corn liquefactions (30% solids) using either archaeal or bacterial amylases. Size exclusion chromatography results (refractive index detector (RID) signal) are shown in function of the corn liquefaction time (minutes).

FIG. 2 provides the dextrose equivalent (in %) of liquefaction medium in function of the different enzyme preparations used during the process.

FIG. 3 provides the viscosity profiles (in cP at 85° C.) of 35 g liquefaction medium in function of the different enzyme preparations used during the process and time (in seconds).

FIG. 4 provides the final ethanol yield (% weight/volume) per solids of lab-scale fermentations in function of the different enzyme preparations used to provide the liquefied mash.

FIG. 5 provides the viscosity profiles (in cP at 85° C.) of 35 g liquefaction medium in function of the different enzyme preparations used during the process and time (in seconds).

FIG. 6 provides the dextrose equivalent (in %) of liquefaction medium in function of the different enzyme preparations used during the process.

FIG. 7 provides the size profile of corn liquefactions (33% solids) using archaeal, bacterial amylases or combinations thereof. Size exclusion chromatography results (refractive index detector (RID) signal) are shown in function of the corn liquefaction time (minutes) and types of preparations used.

FIG. 8 provides the final ethanol yield (% weight/volume) per solids of lab-scale fermentations in function of the weight percentage of the different enzyme preparations used to provide the liquefied mash.

FIG. 9 provides the final ethanol yield (% weight/volume) of lab-scale fermentations in function of the weight percentage of the different enzyme preparations used to provide the liquefied mash.

FIG. 10 provides alpha-amylase activity (in Ceralpha Units/ml of sample) of various enzyme blends preparations.

FIG. 11 provides alpha-amylase activity (on gelatinized starch) of enzymes expressed in Saccharomyces cerevisiae following an incubation at 85° C. Prior to adding substrate, supernatant was pre-incubated at room temperature or 85° C. Results are provided as absorbance (at 540 nm) when the alpha-amylase activity was determined following an incubation at room temperature (gray bars) and at 85° C. (black bars) of enzymes expressed by the different strains listed.

FIGS. 12A and B provides the relative alpha-amylase activity (on gelatinized starch) of enzymes expressed in Saccharomyces cerevisiae.

FIG. 12A. The results are provided following an incubation at 85° C. in the absence and in the presence of 1, 2 or 5 mM EGTA. Results are provided as the relative activity (%) in function of the amount of EGTA used (1 mM EGTA: black bars; 2 mM EGTA: grey bars; and 5 mM EGTA: white bars).

FIG. 12B. The results are provided following an incubation at 85° C. in the absence and in the presence of EGTA and/or CaCl₂). Results are provided as the relative activity (%) in function of the amount of EGTA/CaCl₂) used (1 mM EGTA/no CaCl₂): black bars; 1 mM EGTA/1 mM CaCl₂): diagonally-hatched bars; and 1 mM CaCl₂)/no EGTA: grey bars).

FIG. 13 provides alpha-amylase activity (on gelatinized starch) of enzymes expressed in Saccharomyces cerevisiae following an incubation at a temperature at 85° C. Prior to adding substrate, whole cell culture lysate was pre-incubated at temperatures between 75° C. and 100° C.

FIG. 14 provides the relative alpha-amylase activity (on gelatinized starch) of enzymes expressed in Saccharomyces cerevisiae following an incubation at 85° C. in the absence and in the presence of 1 mM EGTA.

FIG. 15 provides alpha-amylase activity (on gelatinized starch) of enzymes expressed in Komagataella phaffii following an incubation at a temperature at 85° C. Results are shown as the absorbance at 540 nm in function of the strain used to generate the enzyme.

FIGS. 16A and 16B provides alpha-amylase activity (on gelatinized starch) of enzymes expressed in Saccharomyces cerevisiae or Bacillus subtilis following an incubation at a temperature at 85° C. Results are shown as the absorbance at 540 nm in function of the strain used to generate the enzyme (or the control strain).

FIG. 16A. The results are provided for archaeal alpha-amylases expressed in Saccharomyces cerevisiae or Bacillus subtilis strains.

FIG. 16B. The results are provided for bacterial alpha-amylases expressed in Saccharomyces cerevisiae or Bacillus subtilis strains. Prior to adding substrate, supernatant was pre-incubated at room temperature (gray bars) or at 85° C. (black bars).

### DETAILED DESCRIPTION

The present disclosure provides alpha-amylases particularly suited for the liquefaction of starch that is present in a biomass. In some embodiments, the present disclosure provides variant polypeptides of archaeal and bacterial alpha-amylases having improved biological properties. These variant polypeptides can be used on their own or in the compositions/preparations/combinations described herein.

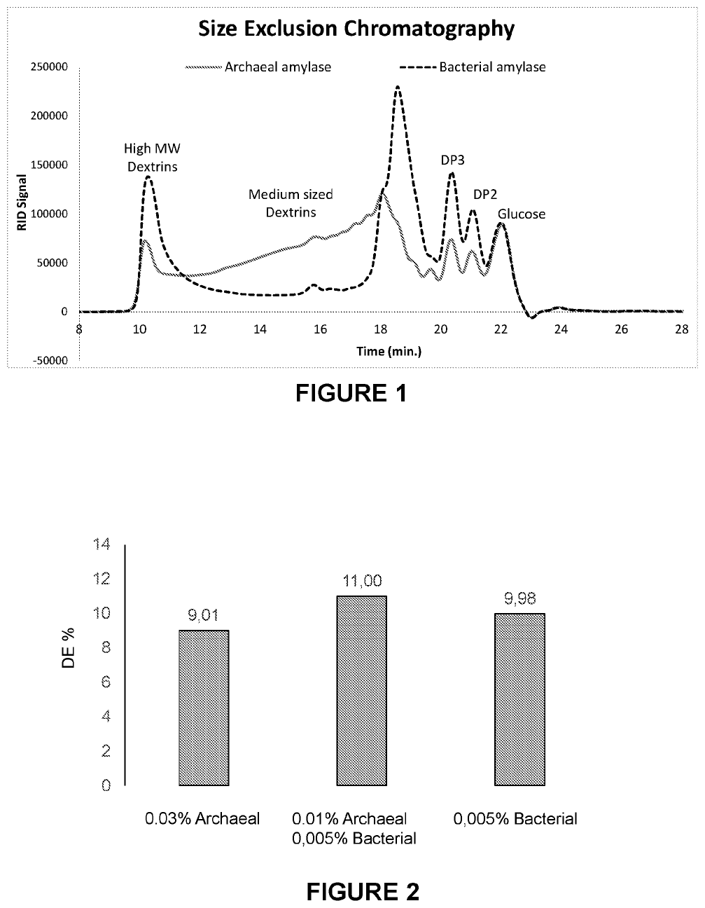

In other embodiments, the present disclosure concerns compositions or preparations combining at least two distinct α-amylases: at least one from an archaeal origin (e.g., an archaeal alpha-amylase being derived from an archaea) and at least one from a bacterial origin (e.g., a bacterial alpha-amylase being derived from a bacteria). Without wishing to be bound to theory, the enzyme combinations described herein achieve, during liquefaction, an optimized dextrin size profile which allow achieving, during liquefaction, a reduction in viscosity and, during a subsequent fermentation, a higher yield. FIG. 1 provides size exclusion chromatograms of corn biomass being liquefied using an archaeal alpha-amylase and another being liquefied using a bacterial alpha-amylase. As shown in FIG. 1, archaeal alpha-amylases have an increased ability (when compared to bacterial alpha-amylases) to hydrolyze high molecular weight dextrins into medium sized molecular weight dextrins. Such medium sized molecular weight dextrins are more efficiently hydrolyzed, during the fermentation, by glucoamylases and can thus further increase the fermentation yield by reducing the high molecular weight starch that is less enzyme accessible. The reduction of high molecular weight starch also prevents retrogradation upon cooling, thereby reducing the resistant starch which by definition is resistant to enzymatic action. The archaeal and bacterial amylases combines the improved saccharification of archaeal amylases with the viscosity reducing benefits of bacterial amylases. As also shown in FIG. 1, bacterial alpha-amylases have improved exo-activity (when compared to archaeal alpha-amylases) which enhances their ability to disrupt swollen starch granules during liquefaction, thereby enhancing the viscosity reduction kinetics which is evidenced by the higher levels of low DP sugars (such as DP4, DP3, DP2 and glucose) which is understood to reduce viscosity during the fermentation.

Polypeptides having α-amylase activity (also referred to as α-amylases; EC 3.2.1.1) are capable of hydrolyzing starch to maltose and dextrins. By acting at random locations along the starch chain, α-amylases break down long-chain carbohydrates, ultimately yielding, dextrins. maltotriose, maltose and smaller chain dextrins from amylose, or maltose, glucose and “limit dextrin” from amylopectin. While alpha-amylases are usually known for exhibiting endoenzyme activity (e.g., the ability to cleave interior glucose α-1,4 bonds), some alpha-amylases can exhibit exoenzyme activity (e.g., the ability to cleave exterior/terminal glucose α-1,4 bonds) and even debranching activity (e.g., the ability to cleave glucose α-1,6 bonds).

Archaeal alpha-amylases usually exhibit a strict endoenzyme activity. In some embodiments, at least 90, 91, 92, 93, 94, 95, 96, 97, 98 or 99% of the alpha-amylase activity associated with the archaeal alpha-amylase is endoenzyme activity. In some further embodiments, 100% of the alpha-amylase activity associated with the archaeal alpha-amylase is endozyme activity. In yet another embodiment, no more than 10, 9, 8, 7, 6, 5, 4, 3, 2, or 1% of the alpha-amylase activity associate with the archaeal alpha-amylase is exoenzyme activity. In yet additional embodiments, the archaeal alpha-amylase does not exhibit any measurable exoenzyme activity.

Archaeal alpha-amylases usually include a single starch-binding domain (e.g., a combination of amino acid residues responsible for physically locating with the starch molecule that is intended or being hydrolyzed with the catalytic domain of the alpha-amylase). Archaeal alpha-amylases also usually include a single surface binding site (e.g., a combination of amino acid residues responsible for physically locating with the starch molecule that is intended or being hydrolyzed outside the catalytic domain of the alpha-amylase). Archaeal alpha-amylases also usually exhibit a debranching activity. Archaeal alpha-amylases can exhibit temperature stability at temperatures equal to or higher than 100° C. Archaeal alpha-amylases can exhibit pH stability over a range of pH between 4.5 and 10. While archaeal alpha-amylases are usually slower than bacterial alpha-amylases to reduce the viscosity of a starch slurry, they usually generate a more distributed dextrin profile than bacterial alpha-amylases (as shown in FIG. 1 for example).

In an embodiment, archaeal alpha-amylases, after liquefaction (which can be, in some embodiments, initially conducted on a liquefaction medium comprising a corn slurry having 30-33% solids), generate an hydrolyzed liquefaction medium comprising less than 50%, less than 40%, less than 30% or even less than 20% of DP1, DP2 and DP3 dextrins, when compared to an hydrolyzed liquefaction medium obtained under similar conditions with bacterial alpha-amylases. In some embodiments, archaeal alpha-amylases, after liquefaction (which can be, in some embodiments, initially conducted on a liquefaction medium comprising a corn slurry having 30-33% solids), generate an hydrolyzed liquefaction medium comprising between 50% and 20% less DP1, DP2 and DP3 dextrins, when compared an hydrolyzed liquefaction medium obtained under similar conditions with bacterial alpha-amylases.

In an embodiment, archaeal alpha-amylases, after liquefaction (which can be, in some embodiments, initially conducted on a liquefaction medium comprising a corn slurry having 30-33% solids), generate an hydrolyzed liquefaction medium comprising 95, 94, 93, 92, 90, 89, 88, 87, 86, 85% or less dextrose equivalent when compared to an hydrolyzed liquefaction medium obtained under similar conditions with bacterial alpha-amylases.

In an embodiment, archaeal alpha-amylases, during the first 5-20 minutes of the liquefaction process (which can be, in some embodiments, initially conducted on a liquefaction medium comprising a corn slurry having 30-33% solids), generate an hydrolyzed liquefaction medium having a maximal viscosity at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2 or higher than the maximal viscosity of an hydrolyzed liquefaction medium obtained under similar conditions (including using a similar dose) with bacterial alpha-amylases.

Bacterial alpha-amylases usually exhibit both an exozyme and an endoenzyme activity. In some embodiments, at least 50, 55, 60, 65, 70, 75, 80, 85, 90, 91, 92, 93, 94 or 95% of the alpha-amylase activity associated with the bacterial alpha-amylase is endoenzyme activity. In some embodiments, at least 5, 6, 7, 8, 9, 10, 15, 20, 25, 30, 35, 40, 45 or 50% of the alpha-amylase activity associated with the bacterial alpha-amylase is exoenzyme activity. In some further embodiments, no more than 50, 45, 40, 35, 30, 25, 20, 15, 10, 9, 8, 7, 6, or 5% of the alpha-amylase activity associated with the bacterial alpha-amylase is exoenzyme activity. Bacterial alpha-amylases usually include more than one starch-binding domain (e.g., a combination of amino acid residues, responsible for physically locating with the starch molecule that is intended or being hydrolyzed with the catalytic domain of the alpha-amylase). In some embodiments, the bacterial alpha-amylases has at least 2, 3, 4 or more starch-binding domains. Bacterial alpha-amylases also usually include more than one single surface binding site (e.g., a combination of amino acid residues, responsible for physically locating with the starch molecule that is intended or being hydrolyzed outside the catalytic domain of the alpha-amylase). In some embodiments, bacterial alpha-amylases has at least 2, 3, 4 or more surface binding sites. Bacterial alpha-amylases also usually fail to exhibit a debranching activity (e.g., the ability to cleave glucose α-1,6 bonds). Bacterial alpha-amylases can exhibit temperature stability at temperatures equal to or lower than 95° C. Bacterial alpha-amylases can exhibit pH stability over a range of pH between 5 and 7. While bacterial alpha-amylases are usually faster than bacterial alpha-amylases to reduce the viscosity of a starch slurry, they usually generate a biphasic dextrin profile (e.g., pools of small dextrins and high-molecular weight starches) when compared to the profile obtained with the archaeal alpha-amylases.

In an embodiment, bacterial alpha-amylases, after liquefaction (which can be, in some embodiments, initially conducted on a liquefaction medium comprising a corn slurry having 30-33% solids), generate an hydrolyzed liquefaction medium comprising more than 20%, more than 30%, more than 40% or even more than 50% of DP1, DP2 and DP3 dextrins, when compared to an hydrolyzed liquefaction medium obtained under similar conditions with archaeal alpha-amylases. In some embodiments, bacterial alpha-amylases, after liquefaction (which can be, in some embodiments, initially conducted on a liquefaction medium comprising a corn slurry having 30-33% solids), generate an hydrolyzed liquefaction medium comprising between 20% and 50% more DP1, DP2 and DP3 dextrins, when compared an hydrolyzed liquefaction medium obtained under similar conditions with archaeal alpha-amylases.

In an embodiment, bacterial alpha-amylases, after liquefaction (which can be, in some embodiments, initially conducted on a liquefaction medium comprising a corn slurry having 30-33% solids), generate an hydrolyzed liquefaction medium comprising at least 5, 6, 7, 8, 9, 10, 11, 12, 13, 14, 15% or more dextrose equivalent when compared to an hydrolyzed liquefaction medium obtained under similar conditions with archaeal alpha-amylases. In some embodiments, bacterial alpha-amylases, after liquefaction (which can be, in some embodiments, initially conducted on a liquefaction medium comprising a corn slurry having 30-33% solids), generate an hydrolyzed liquefaction medium comprising between 5 and 15% dextrose equivalent when compared to an hydrolyzed liquefaction medium obtained under similar conditions with archaeal alpha-amylases

In an embodiment, bacterial alpha-amylases, after liquefaction (which can be, in some embodiments, initially conducted on a liquefaction medium comprising a corn slurry having 30-33% solids), generate an hydrolyzed liquefaction medium having a maximal viscosity at least 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 or lower than the maximal viscosity of an hydrolyzed liquefaction medium obtained under similar conditions with archaeal alpha-amylases.

Irrespective of their origin, polypeptides exhibiting alpha-amylase activity can be identified by various ways by the person skilled in the art. For example, the alpha-amylase activity of a polypeptide can be determined directly by measuring the amount of reducing sugars generated by the polypeptide in an assay in which raw or gelatinized starch is used as the starting material. The α-amylase activity of a polypeptide can be measured indirectly, for example, by measuring the amount of reducing sugars generated by the polypeptide in an assay in which starch (raw or gelatinized) is used as the starting material. The determination of alpha-amylase activity can be performed, in some embodiments, after a heat challenge to determine the stability of an enzyme at a certain temperature. The determination of alpha-amylase activity can be performed, in some embodiments, in the absence and in the presence of a chelating agent (EGTA, TPEN, DTPA or phytic acid for example) to determine the relative alpha-amylase activity of an enzyme.

In some embodiments, the present disclosure concerns the use of a combination of at least two distinct α-amylases: one or more α-amylase from an archaea (e.g., an archaeal α-amylase) and one or more α-amylase from a bacteria (e.g., a bacterial α-amylase). The one or more archaeal α-amylases can comprise a known archaeal α-amylase, or be a variant of an archaeal α-amylase (exhibiting α-amylase activity). The one or more bacterial α-amylase can comprise a known bacterial α-amylase, or be a variant of a bacterial α-amylase (exhibiting α-amylase activity).

A variant archaeal or bacterial α-amylase comprises at least one amino acid difference (substitution, addition or deletion) when compared to the amino acid sequence of the wild-type archaeal/bacterial α-amylase. The variant archaeal/bacterial α-amylase has at least 50%, 60%, 70%, 80%, 85%, 90%, 91%, 92%, 93%, 94%, 95%, 96%, 97%, 98% or 99% identity to the amino acid sequence of the wild-type archaeal/bacterial α-amylase. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press. NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant archaeal/bacterial α-amylase also exhibits alpha-amylase activity. In an embodiment, the variant archaeal/bacterial α-amylase exhibits at least 50%, 60%, 70%, 80%, 90%, 91%, 92%, 93%, 94%, 95%, 96%, 97%, 98%, 99%, 100%, 110%, 120%, 130%, 140%, 150% or more of alpha-amylase activity when compared to the wild-type corresponding archaeal/bacterial α-amylase. In some embodiment, when the wild-type archaeal/bacterial α-amylase exhibits dependence to a metallic ion, the variant archaeal or bacterial α-amylase also exhibits dependence to a metallic ion, but to a lesser degree. In some embodiments, when the wild-type archaeal/bacterial α-amylase exhibits stability at elevated temperatures (e.g., a thermostable wild-type archaeal/bacterial α-amylase), the variant archaeal or bacterial α-amylase also exhibits α-amylase activity after having been exposed to elevated temperatures (such as, for example, a temperature of about 60° C., 65° C., 70° C., 75° C., 80° C., 85° C., 90° C., 95° C., 99° C., or more). In some embodiment, when the wild-type archaeal/bacterial α-amylase exhibits susceptibility towards chelation, the variant archaeal or bacterial α-amylase also can exhibit some susceptibility towards chelation, but to a lesser degree.

The variant archaeal/bacterial α-amylase described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant archaeal/bacterial α-amylase can be also be a conservative variant or an allelic variant. As used herein, a conservative variant refers to alterations in the amino acid sequence that do not adversely affect the biological functions of the archaeal or bacterial α-amylase (e.g., hydrolysis of starch). A substitution, insertion or deletion is said to adversely affect the polypeptide when the altered sequence prevents or disrupts a biological function associated with the archaeal or bacterial α-amylase (e.g., the hydrolysis of starch into maltose and dextrins). For example, the overall charge, structure or hydrophobic-hydrophilic properties of the polypeptide can be altered without adversely affecting a biological activity. Accordingly, the amino acid sequence can be altered, for example to render the polypeptide more hydrophobic or hydrophilic or adding a signal sequence, without adversely affecting the biological activities of the α-amylase.

In some embodiments, a variant (which may also be referred to as a fragment) of an archaeal/bacterial α-amylase comprises at least one less amino acid residue when compared to the amino acid sequence of the wild-type corresponding archaeal/bacterial α-amylase polypeptide or variant (described herein) and still possess the enzymatic activity of the full-length wild-type α-amylase (in an embodiment, at the same temperature as the full-length α-amylase). In some embodiments, a fragment can correspond to the archaeal/bacterial α-amylase or a variant thereof described herein to which the signal peptide sequence has been removed. The fragment can be, for example, a truncation of one or more amino acid residues at the amino terminus, the carboxy terminus or both termini of the archaeal/bacterial α-amylase or variant. Alternatively or in combination, the fragment can be generated from removing one or more internal amino acid residues. In an embodiment, the archaeal/bacterial alpha-amylase fragment has at least 100, 150, 200, 250, 300, 350, 400, 450 or more consecutive amino acids of the wild-type corresponding archaeal/bacterial-amylase or the variant.

The archaeal/bacterial α-amylase fragment has at least 50%, 60%, 70%, 80%, 85%, 90%, 91%, 92%, 93%, 94%, 95%, 96%, 97%, 98% or 99% identity to the amino acid sequence of the wild-type corresponding archaeal/bacterial α-amylase or the variant. In an embodiment, the fragment of the archaeal/bacterial α-amylase exhibits at least 50%, 60%, 70%, 80%, 90%, 91%, 92%, 93%, 94%, 95%, 96%, 97%, 98%, 99%, 100%, 110%, 120%, 130%, 140%, 150% or more of the activity of wild-type corresponding archaeal/bacterial α-amylase. In some embodiment, when the wild-type archaeal/bacterial α-amylase exhibits dependence to a metallic ion, the archaeal or bacterial α-amylase fragment also exhibits dependence to a metallic ion, but to a lesser degree. In some embodiments, when the wild-type archaeal/bacterial α-amylase exhibits stability at elevated temperatures (e.g., a thermostable wild-type archaeal/bacterial-amylase), the archaeal or bacterial α-amylase fragment also exhibits α-amylase activity after having been exposed to elevated temperatures (such as, for example, a temperature of about 60° C., 65° C., 70° C., 75° C., 80° C., 85° C., 90° C., 95° C., 99° C., or more). In some embodiment, when the wild-type archaeal/bacterial α-amylase exhibits susceptibility towards chelation, the archaeal or bacterial α-amylase fragment also can exhibit some susceptibility towards chelation, but to a lesser degree.

The alpha-amylases, their associated variants as well as the combinations described herein can be used in combination (or admixed with), in some embodiments, one or more additional lytic enzyme (e.g., an enzyme involved in the cleavage or hydrolysis of its substrate). For example, the enzyme combination can include one or more thermostable lytic enzymes. In still another embodiment, the lytic enzyme can be a glycoside hydrolase. In the context of the present disclosure, the term “glycoside hydrolase” refers to an enzyme involved in carbohydrate digestion, metabolism and/or hydrolysis, including amylases, cellulases. hemicellulases, cellulolytic and amylolytic accessory enzymes, inulinases, levanases, trehalases, pectinases, xylanases, xylosidases, arabinofuranosidases, galactosidases, endoglucanases and/or pentose sugar utilizing enzymes. In another embodiment, the lytic enzyme can be a protease. In the context of the present disclosure, the term “protease” refers to an enzyme involved in protein digestion, metabolism and/or hydrolysis. In yet another embodiment, the enzyme can be an esterase. In the context of the present disclosure, the term “esterase” refers to an enzyme involved in the hydrolysis of an ester from an acid or an alcohol, including phosphatases such as phytases.

### Archaeal Alpha-Amylase

The enzyme combination of the present disclosure comprises at least one archaeal alpha-amylase which is intended to be used with at least one bacterial alpha-amylase during the liquefaction of a biomass. The present disclosure also provides a variant polypeptide which has been obtained from a wild-type parental archaeal alpha-amylse. As used in the context of the present disclosure, an archaeal alpha-amylase refers to a polypeptide having alpha-amylase activity which is natively expressed in an archaea. The archaeal alpha-amylase is considered “derived” from an archaea because the nucleic acid sequence encoding it was obtained or copied from an archaea and was optionally modified prior to its introduction in the heterologous nucleic acid (intended to be introduced in the recombinant microbial host cell).

The nucleic acid molecule encoding the archaeal α-amylase is necessarily heterologous with respect to the recombinant microbial host cell, it is either not natively present in the recombinant microbial host cell or is present at a non-native location in the recombinant microbial host cell. In an embodiment, the archaeal α-amylase can exhibit activity at elevated temperatures (e.g., be thermostable).

The at least one archaeal α-amylase comprises a polypeptide having α-amylase activity and be derived from any archaea. In an embodiment, the archaeal α-amylase can be derived from the genus Thermococcus sp. In yet a further embodiment, the archaeal α-amylase can be derived from the species Thermococcus hydrothermalis. In such embodiment, the archaeal α-amylase can have the amino acid sequence of SEQ ID NO: 1, 13, 19, 23, 24, 25, 30, 54, 55, 56, 57, 58, 59, 60, 65, 67, or 70, or be a variant thereof (including a fragment thereof) having α-amylase activity. In yet a further embodiment, the archaeal α-amylase can be derived from the species Thermococcus gammatolerans. In such embodiment, the archaeal α-amylase can have the amino acid sequence of SEQ ID NO: 7, 10 or 15, or be a variant thereof (including a fragment thereof) having α-amylase activity. In yet a further embodiment, the archaeal α-amylase can be derived from the species Thermococcus thioreducens. In such embodiment, the archaeal α-amylase can have the amino acid sequence of SEQ ID NO: 8, 11 or 17, or be a variant thereof (including a fragment thereof) having α-amylase activity. In yet a further embodiment, the archaeal α-amylase can be derived from the species Thermococcus eurythermalis. In such embodiment, the archaeal α-amylase can have the amino acid sequence of SEQ ID NO: 9, 12 or 18, or be a variant thereof (including a fragment thereof) having α-amylase activity or be a fragment thereof having α-amylase activity.

In an embodiment, the archaeal α-amylase can be derived from the genus Pyrococcus sp. In yet a further embodiment, the archaeal α-amylase can be derived from the species Pyrococcus furiosus. In such embodiment, the archaeal α-amylase can have the amino acid sequence of SEQ ID NO: 2, 14, 16, 20, 21, 22, 26, or 66, or be a variant thereof (including a fragment thereof) having α-amylase activity.

In yet another embodiment, the enzymatic combination of the present disclosure comprises at least two distinct archaeal α-amylases, and in a further embodiment, each archaeal-amylase being derived from a different genus and/or species. In still another specific embodiment, the enzymatic combination of the present disclosure comprises a first archaeal α-amylase derived from a Thermococcus sp. and a second archaeal α-amylase derived from a Pyrococcus sp. In still a further embodiment, the enzymatic combination of the present disclosure comprises a first archaeal α-amylase derived from Thermococcus hydrothermalis and a second archaeal α-amylase derived from a Pyrococcus furiosus. In yet a further embodiment, the enzymatic combination of the present disclosure comprises a first archaeal α-amylase having the amino acid sequence of SEQ ID NO: 1 (a variant thereof or a fragment thereof) and a second archaeal α-amylase having the amino acid sequence of SEQ ID NO: 2 (a variant thereof or a fragment thereof). In embodiments when the enzymatic combination comprises at least two distinct archaeal α-amylases, it can be provided with one or more bacterial α-amylases.

In yet another embodiment, the enzymatic combination of the present disclosure comprises at least three distinct archaeal α-amylases. In still another specific embodiment, the enzymatic combination of the present disclosure comprises a first and a second archaeal α-amylase derived from a Thermococcus sp. and a third archaeal α-amylase derived from a Pyrococcus sp. In still a further embodiment, the enzymatic combination of the present disclosure comprises a first and a second archaeal α-amylase derived from Thermococcus hydrothermalis and a third archaeal α-amylase derived from a Pyrococcus furiosus. In yet a further embodiment, the enzymatic combination of the present disclosure comprises a first archaeal α-amylase having the amino acid sequence of SEQ ID NO: 25 (or a variant thereof). a second archaeal α-amylase having the amino acid sequence of SEQ ID NO: 30 (or a variant thereof) and a third archaeal α-amylase having the amino acid sequence of SEQ ID NO: 26 (or a variant thereof). In embodiments when the enzymatic combination comprises at least three distinct archaeal α-amylases, it can be provided with one or more bacterial α-amylases.

The enzymatic combination of the present disclosure includes at least one archaeal α-amylase. optionally with more than one bacterial α-amylases. In some embodiments, the enzymatic combination of the present disclosure can comprise at least two, three, four or five archaeal α-amylases, optionally with more than one bacterial α-amylases.

In yet another embodiment, the enzymatic combination of the present disclosure comprises a first archaeal α-amylase and a second bacterial α-amylase. In still another specific embodiment, the enzymatic combination of the present disclosure comprises the first archaeal α-amylase derived from a Thermococcus sp. and the second bacterial α-amylase derived from a Geobacillus sp. In still a further embodiment, the enzymatic combination of the present disclosure comprises the first archaeal α-amylase derived from Thermococcus hydrothermalis and the second α-amylase derived from Geobacillus stearothermophilus. In yet a further embodiment, the enzymatic combination of the present disclosure comprises a first archaeal α-amylase having the amino acid sequence of SEQ ID NO: 25 (or a variant thereof) and a second bacterial α-amylase having the amino acid sequence of SEQ ID NO: 28 or 29 (or a variant thereof).

In an embodiment, the archaeal alpha-amylases of the present disclosure can be provided from a recombinant microbial host cell having expressed it. In such embodiment, the archaeal alpha-amylase can be provided in association with the recombinant microbial host cell having expressed it (e.g., a cell-associated form for example). In some embodiments, the recombinant microbial host cell can be living, inactivated (at least in part) or completely inactivated (dead). Alternatively or in combination, the archaeal alpha-amylase can be provided in a secreted form with the recombinant microbial host cell having expressed it.

In an embodiment, the archaeal alpha-amylase (or combination thereof) can be provided as a product derived from a recombinant microbial host cell and can be referred to as a microbial product. In such embodiment, the microbial product comprises, besides the archaeal alpha-amylase (or variant thereof), a component of the recombinant microbial host cell having expressed it. The “component of the recombinant microbial host cell” can be an intracellular component and/or a component associated with the microbial host cell's wall or membrane. The “component of the recombinant microbial host cell” can include a protein, a peptide or an amino acid, a carbohydrate and/or a lipid. The “component of the recombinant microbial host cell” can include a microbial host cell organelle. The “component of the recombinant microbial host cell” can be a microbial extract, such as, for example, a bacterial extract, a fungal extract or a yeast extract. The composition comprising the archaeal alpha-amylase (or combination thereof) and the component of the recombinant microbial host cell can be an inactive composition (e.g., none of the recombinant microbial host cells are alive), a semi-active or inactivated composition (e.g., some of the recombinant microbial host cells are alive) or an active composition (e.g., most of the recombinant microbial host cells are alive). The composition can be a liquid or a solid (e.g., dried, frozen and/or lyophilized) product. Inactivated yeast products include, but are not limited to a yeast extract. Active/semi-active yeast products include, but are not limited to, a cream yeast, an instant dried yeast or an active-dried yeast. Inactivated bacterial products, include but are not limited to a bacterial extract. An active/semi-active bacterial products include, but are not limited to, bacterial concentrates. Inactivated fungal products, include but are not limited to a fungal extract. An active/semi-active fungal products include, but are not limited to, fungal concentrates. In some embodiments, the yeast product is a yeast extract produced from recombinant yeast host cells expressing the at least one archaeal alpha-amylase. In some additional embodiments, the bacterial product is a bacterial extract produced from the recombinant microbial host cells expressing the at least one archaeal alpha-amylase. In some additional embodiments, the fungal product is a fungal extract produced from the recombinant microbial host cells expressing the at least one archaeal alpha-amylase.

In an embodiment, the archaeal alpha-amylase is provided as a product derived from a yeast and can be referred to as an archaeal alpha-amylase yeast product. In additional embodiments, the archaeal alpha-amylase yeast product can be provided as an inactivated yeast product which can, in some additional embodiments, be obtained by homogenizing a yeast having expressed the archaeal alpha-amylase (or a combination of archaeal alpha-amylases). In further embodiments, between 0.01-0.03% of dry cell weight/weight of dry corn solids (dcw/w) the archaeal alpha-amylase yeast product can be added to a liquefaction medium (such as a slurry). In additional embodiments, no more than 0.03, 0.02 or 0.01% of dcw/w of the archaeal alpha-amylase yeast product can be added to the liquefaction medium (such as the slurry).

In an embodiment, the at least one archaeal alpha-amylase can be provided in a semi-purified or in a substantially purified form. As used in the context of the present disclosure, the expression “semi-purified form” refers to the fact that the at least one archaeal alpha-amylase has been physically dissociated, at least in part, from the components of the recombinant microbial host cell expressing same. The expression “substantially purified form” refers to the fact that the at least one archaeal alpha-amylases has been physically dissociated from the majority of the components of the recombinant microbial host cell expressing same. In an embodiment, a composition comprising archaeal alpha-amylase(s) in a substantially purified form is at least 90%, 95%, 96%, 97%, 98% or 99% pure. In some embodiments, the composition comprising archaeal alpha-amylase(s) lacks a detectable amount of deoxyribonucleic acids from the microbial host cell used to express it. In some embodiments, an archaeal alpha-amylase provided in a substantially purified form may be provided as part of a mixture which can include additional polypeptides having alpha-amylase activity and/or other lytic activity.

In another embodiment, the archaeal alpha-amylases is provided in a substantially purified form. In some embodiments, the archaeal alpha-amylase is provided as a commercial preparation and is not obtained from the metabolism of a yeast host cell. In some embodiments, 0.03% weight of enzyme/weight of dry corn solids (w/w) the archaeal alpha-amylase preparation can be added to a liquefaction medium (such as a slurry). In an embodiment, the archaeal alpha-amylase comprises at least one or a plurality of commercially available archaeal alpha-amylases, such as, for example, Syngenta Enogen Corn™. In alternative embodiments, the archaeal alpha-amylase preparation exclude the Fuelzyme products (such as, for example, BASF Fuelzyme™ and BASF Fuelzyme 650™, and the polypeptide having the amino acid sequence of SEQ ID NO: 42).

### Bacterial Alpha-Amylase

As used in the context of the present disclosure, a bacterial α-amylase refers to a polypeptide having alpha-amylase activity which is natively expressed in a bacteria. The bacterial α-amylase is considered “derived” from a bacteria because the nucleic acid sequence encoding it was obtained or copied from a bacteria and was optionally modified prior to its introduction in the heterologous nucleic acid (intended to be introduced in the recombinant microbial host cell). The nucleic acid molecule encoding the bacterial α-amylase is necessarily heterologous with respect to the recombinant microbial host cell, it is either not natively present in the recombinant microbial host cell or is present at a non-native location in the recombinant microbial host cell. In an embodiment, the at least one bacterial α-amylase can exhibit activity at elevated temperatures (e.g., be thermostable).

The at least one bacterial α-amylase comprises a polypeptide having α-amylase activity and be derived from any bacteria. In an embodiment, the bacterial α-amylase can be derived from the genus Geobacillus sp. In yet a further embodiment, the archaeal α-amylase can be derived from the species Geobacillus stearothermophilus. In such embodiment, the bacterial α-amylase can have the amino acid sequence of SEQ ID NO: 3, 4, 5, 6, 27, 28, 29, 38, 39, 43, 44, 45, 46, 47, 48, 49, 50, 51, 52, 53, 61, 62, 63, 64, 68, 69, or 71, or be a variant thereof (including a fragment thereof) having α-amylase activity.

The combination of the present disclosure comprises at least one bacterial alpha-amylase. In yet another embodiment, the enzymatic combination of the present disclosure comprises at least two distinct bacterial α-amylases. In such embodiment, each bacterial α-amylase can be derived from a different genus and/or species. In yet another embodiment, the enzymatic combination of the present disclosure comprises at least three or more distinct bacterial α-amylases. In embodiments when the enzymatic combination comprises at least two distinct bacterial α-amylase, it can be provided with more than one archaeal α-amylase.

The enzymatic combination of the present disclosure includes at least one bacterial α-amylase, optionally with more than one archaeal α-amylases. In some embodiments, the enzymatic combination of the present disclosure can comprise at least two, three, four or five bacterial α-amylases, optionally with more than one archaeal α-amylase.

In yet another embodiment, the enzymatic combination of the present disclosure comprises a first archaeal α-amylase and a second bacterial α-amylase. In still another specific embodiment, the enzymatic combination of the present disclosure comprises the first archaeal α-amylase derived from a Thermococcus sp. and the second bacterial α-amylase derived from a Geobacillus sp. In still a further embodiment, the enzymatic combination of the present disclosure comprises the first archaeal α-amylase derived from Thermococcus hydrothermalis and the second α-amylase derived from Geobacillus stearothermophilus. In yet a further embodiment, the enzymatic combination of the present disclosure comprises a first archaeal α-amylase having the amino acid sequence of SEQ ID NO: 25 (or a variant) and a second bacterial α-amylase having the amino acid sequence of SEQ ID NO: 28 or 29 (or a variant thereof).

In an embodiment, the bacterial alpha-amylase (or combination thereof) can be provided in combination with a recombinant microbial host cell (as described herein) having expressed it. In such embodiment, the bacterial alpha-amylase can be provided in association with the recombinant microbial host cell having expressed it (e.g., in a tethered and/or intracellular form). Alternatively or in combination, the bacterial alpha-amylase can be provided in a secreted form with the recombinant microbial host cell having expressed it. In some embodiments, the recombinant microbial host cell can be living, inactivated (at least in part) or completely inactivated (dead).

In an embodiment, the bacterial alpha-amylase (or combination thereof) can be provided as a product derived from a recombinant microbial host cell and can be referred to as a microbial product. In such embodiment, the microbial product comprises, besides the bacterial alpha-amylase (or combination thereof), a component of the recombinant microbial host cell having expressed it. The “component of the recombinant microbial host cell” can be an intracellular component and/or a component associated with the microbial host cell's wall or membrane. The “component of the recombinant microbial host cell” can include a protein, a peptide or an amino acid, a carbohydrate and/or a lipid. The “component of the recombinant microbial host cell” can include a microbial host cell organelle. The “component of the recombinant microbial host cell” can be a microbial extract, such as, for example, a bacterial extract, a fungal extract or a yeast extract. The composition comprising the bacterial alpha-amylase (or combination thereof) and the component of the recombinant microbial host cell can be an inactive composition (e.g., none of the recombinant microbial host cells are alive), a semi-active or inactivated composition (e.g., some of the recombinant microbial host cells are alive) or an active composition (e.g., most of the recombinant microbial host cells are alive). The composition can be a liquid or a solid (e.g., dried, frozen and/or lyophilized) product. Inactivated yeast products include, but are not limited to a yeast extract. Active/semi-active yeast products include, but are not limited to, a cream yeast, an instant dried yeast or an active-dried yeast. Inactivated bacterial products, include but are not limited to a bacterial extract. An active/semi-active bacterial products include, but are not limited to, bacterial concentrates. Inactivated fungal products, include but are not limited to a fungal extract. An active/semi-active fungal products include, but are not limited to, fungal concentrates. In some embodiments, the yeast product is a yeast extract produced from recombinant yeast host cells expressing the at least one bacterial alpha-amylase. In some additional embodiments, the bacterial product is a bacterial extract produced from the recombinant microbial host cells expressing the at least one bacterial alpha-amylase. In some additional embodiments, the fungal product is a fungal extract produced from the recombinant microbial host cells expressing the at least one bacterial alpha-amylase.

In an embodiment, the bacterial alpha-amylase is provided as a product derived from a yeast and can be referred to as a bacterial alpha-amylase yeast product. In additional embodiments, the bacterial alpha-amylase yeast product can be provided as an inactivated yeast product which can, in some additional embodiments, be obtained by homogenizing a yeast expressing the bacterial alpha-amylase (or a combination of bacterial alpha-amylases). In further embodiments, between 0.01-0.03% of dry cell weight/weight of dry corn solids (dcw/w) the bacterial alpha-amylase yeast product can be added to a liquefaction medium (such as a slurry). In additional embodiments, no more than 0.03, 0.02 or 0.01% of dcw/w of the bacterial alpha-amylase yeast product can be added to the liquefaction medium (such as the slurry). In an embodiment, the at least one bacterial alpha-amylase can be provided in a semi-purified or in a substantially purified form. As used in the context of the present disclosure, the expression “semi-purified form” refers to the fact that the at least one bacterial alpha-amylase has been physically dissociated, at least in part, from the components of the recombinant microbial host cell expressing same. The expression “substantially purified form” refers to the fact that the at least one bacterial alpha-amylase has been physically dissociated from the majority of the components of the recombinant microbial host cell expressing same. In an embodiment, a composition comprising bacterial alpha-amylase(s) in a substantially purified form is at least 90%, 95%, 96%, 97%, 98% or 99% pure. In some embodiments, the composition comprising bacterial alpha-amylase(s) lacks a detectable amount of deoxyribonucleic acids from the microbial host cell used to express it. In some embodiments, a bacterial alpha-amylase provided in a substantially purified form may be provided as part of a mixture which can include additional polypeptides having alpha-amylase activity and/or other lytic activity.

In another embodiment, the bacterial alpha-amylases is provided as a commercial preparation and is not obtained from the metabolism of a yeast host cell. In some embodiments, 0.005-0.015% weight of enzyme/weight of dry corn solids (w/w) the bacterial alpha-amylase preparation can be added to a liquefaction medium (such as a slurry). In an embodiment, the bacterial alpha-amylase comprises at least one or a plurality of commercially available bacterial alpha-amylases, such as, for example, CTE Global AMYL-LP™, CTE Global AMYL-LP Strong™, CTE Global AMYL-LTP™, CTE Global AMYL-LTP+™, CTE Global AMYL-XT™ CTE Global AMYL-XTP+™, DSM Maxamyl HT Ultra™, DuPont Solvamyl ADV™, DuPont Specialty Blend™, DuPont Spezyme Alpha™, DuPont Spezyme CLT, DuPont Spezyme CL WB™, DuPont Spezyme HN™, DuPont Spezyme HT™, DuPont Spezyme HT WB™, DuPont Spezyme RSL™, Lallemand DistilaZyme™, Lallemand DistilaZyme™, Novozyme Flex™ Novozymes Avantec™, Novozymes Avantec Amp™, Novozymes BPX™, Novozymes Fortiva Hemi™, Novozymes Liquozyme™, Novozymes Liquozyme LpH™, Novozymes Liquozyme LpH+™, Novozymes Liquozyme SC 4X™, Novozymes Liquozyme SC DS™, Novozymes Fortiva Revo™, Novozymes Fortiva Revo HPI™, Novozymes Fortiva Revo HPX™, and Novozymes Fortiva Revo HTX™. In alternative embodiments, the bacterial alpha-amylase preparation excludes an alpha amylase derived from Bacillus licheniformis (such as, for example, any one of the Spezyme product). Alpha-amylases derived from Bacillus licheniformis include, without limitations, the polypeptides having the amino acid sequence of SEQ ID NO: 40 and 41.

### Variant Polypeptides Having Improved Alpha-Amylase Activity

The present disclosure provides variants of archaeal and bacterial alpha-amylase having improved properties when compared to their corresponding wild-type parental alpha-amylases. In an embodiment, the polypeptide variants of the present disclosure have higher alpha-amylase activity than their corresponding wild-type parental alpha-amylases. As is it known in the art, alpha-amylase activity can be determined directly (by determining the amount of starch that has been hydrolyzed) or indirectly (by determining the effects/use of the hydrolyzed of starch). In an embodiment, the alpha-amylase activity can be determined directly by using a non-specific assay (such as, for example the DNS (3,5-dinitrosalicylic acid) assay, or the Nelson-Somogyi (NS) assay)) or a specific assay (such as, for example, by using the Ceralpha method or the Phadebas® assay). In another embodiment, the alpha-amylase activity is determined indirectly by assessing the effects of the hydrolyzed starch in the liquefaction medium. This can be done, for example, by determining the viscosity of an hydrolyzed liquefaction medium (e.g., the more alpha-amylase activity, the less viscous the liquefaction medium will be), the amount of dextrose equivalent (DE) of the hydrolyzed liquefaction medium (e.g., the more alpha-amylase activity, the more DE will be present in the hydrolyzed liquefaction medium), the degree of polymerization (DP) profile (e.g., the more alpha-amylase activity, the less high-molecular weight dextrins/the more medium-sized and low-molecular weight dextrins will be present in the hydrolyzed liquefaction medium), and/or the amount of a fermentation product (e.g., ethanol) which can be obtained from an hydrolyzed liquefaction medium (e.g., the more alpha-amylase activity, the more ethanol can be obtained from the fermentation of an hydrolyzed liquefaction medium). In some embodiments, the polypeptide considered of having increased alpha-amylase activity has at least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300% or more α-amylase activity than the α-amylase activity of the corresponding wild-type parental α-amylase.

The present disclosure provides variant polypeptides having alpha-amylase activity exhibiting improved biological properties when compared to the wild-type parental polypeptide from which the variant polypeptide is derived. In one embodiment, the variant polypeptide has improved thermostability when compared to the wild-type parental polypeptide. As it is known in the art, the expression “thermodynamic stability” is understood to refer to the difference in Gibbs' free energy between a folded (or active) and an unfolded (or inactive) state. As it is also known in the art, the expression “kinetic stability” of a polypeptide is understood as the rate at which a polypeptide switched between an active to an inactive state. In some embodiments, the increase in thermodynamic stability/kinetic stability can be observed as an increase in thermostability of the polypeptide. The “thermostability” of a polypeptide can be assessed for example, by determining the residual activity after a heat challenge and/or the melting temperature of the polypeptide. The increase in thermodynamic stability/kinetic stability can be, in embodiments, an increase in specific enzyme activity (e.g., in μmol/mg/min). In some embodiments, the polypeptide exhibits an increase in residual activity of at least 0.5, 0.6, 0.7, 0.8, 0.9, 1, 2, 3, 4-fold or more after a heat challenge at temperatures of 85° C. to 100° C. for 5 min or more, when compared to a corresponding wild-type parental polypeptide. In some embodiments, the polypeptide exhibits an increase in melting temperature of at least of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6° C. or more when measured at pH 5.5, when compared to a corresponding wild-type parental polypeptide.

Alternatively or in combination, the variant polypeptide can be less dependent (than the corresponding wild-type parental alpha-amylase) on the presence of a metallic ion for the structure/function/stability of the enzyme. Some α-amylases are known to bind to metallic ion(s) (e.g., calcium and zinc for example) and such association between the polypeptide and the metallic ion(s) can have an influence on the structure, the biological function and/or the stability of the enzyme. In some embodiments, the variant polypeptides of the present disclosure are “less dependent on the presence of a metallic ion” because they are able to bind with greater affinity and/or avidity the metallic ion(s) and thus maintain their structure/function/stability. Additionally or in combination, the variant polypeptides of the present disclosure are “less dependent on the presence of a metallic ion” because they are able to bind with greater affinity and/or avidity the metallic ion(s) and thus resist chelation of its bound metallic ion(s) in the presence of a chelating agent. In an embodiment, the metallic ion is a calcium ion. In some further embodiments, each variant polypeptide is capable of binding (or is bound to) to at least two calcium ions. In another embodiment, the metallic ion is a zinc ion. In some further embodiments, each variant polypeptide is capable of binding (or is bound to) to at least two zinc ions. In some embodiments, the metallic ions comprise both a calcium ion and a zinc ion. In some further embodiments, each variant polypeptide is capable of binding (or is bound to) to one calcium ion and one zinc ion.

As it is also known in the art, “chelation” refers to the formation of multiple coordination bonds between an organic molecule (also known as a “chelator” or a “chelating agent”) and a metallic ion (such as, for example a calcium ion and/or a zinc ion) leading to sequestration of the metallic ion. While the variant polypeptide itself is, in theory, considered a chelating agent, in the context of the present disclosure, the expression “chelating agent” refers to a chemical or biological entity that is different from the variant polypeptide and is capable of forming multiple coordination bonds with the metallic ion. In some embodiments, the variant alpha-amylase has increased resistance towards one or more chelating agents. This can be especially useful since chelating agents such as phytic acid can be present in liquefaction medium. The ability of an α-amylase to be less dependent on the presence of a metallic ion (which can be conferred, at least in part, by its ability to resist chelation) can be determined, for example, by comparing the biological activity of an α-amylase in the presence and in the absence of a chelating agent.

The ratio or percentage of the α-amylase activity of a polypeptide in the absence vs. in the presence of a chelating agent is referred to as the “relative” α-amylase activity of such polypeptide. It is possible to determine the relative alpha-amylase activity of a test polypeptide susceptible of binding one or more calcium ions using a chelating agent specific to calcium ions, such as, for example, EGTA (Ethyleneglycol-bis(3-aminoethyl)-N, N,N′,N′-tetraacetic Acid, or egtazic acid). It is also possible to determine the relative alpha-amylase activity of a test polypeptide susceptible of binding one or more zinc ions using a chelating agent specific to zinc ions, such as, for example, TPEN (N,N,N′,N′-Tetrakis (2-pyridylmethyl)ethylenediamine) or DTPA (diethylenetriaminepentaacetic acid).

To determine if a test polypeptide is more resistant to chelation compared to its corresponding parental wild-type α-amylase, it is possible to compare the relative α-amylase activity of the test polypeptide with the relative α-amylase activity of the corresponding parental wild-type parental α-amylase in the absence/presence of a chelating agent (EGTA, TPEN or DTPA for example). If the relative α-amylase activity of the test polypeptide is closer to 1.0 (ratio) or 100% (percentage) than the relative α-amylase activity of the corresponding wild-type parental α-amylase, the test polypeptide will be considered to have higher resistance to chelation than the corresponding wild-type parental α-amylase. If the relative α-amylase activity of the test polypeptide is further from 1.0 (ratio) or 100% (percentage) than the relative α-amylase activity of the corresponding wild-type parental α-amylase, the test polypeptide will be considered to having a lower resistance to chelation than the corresponding wild-type parental α-amylase. If the relative α-amylase activity of the test polypeptide is substantially similar to the relative α-amylase activity of the corresponding wild-type parental α-amylase, the test polypeptide will be considered to having a substantially similar resistance towards chelation than the corresponding wild-type parental α-amylase. For example, if chelation is observed, addition of excess (unbound) metallic ion can be used to rescue the activity of the test alpha-amylase. The metallic ion can be provided, in some embodiments, in the form of a salt (CaCl₂) for example). In some embodiments, the test polypeptide is considered to have more resistance to chelation if it has at least 1, 2, 3, 4, 5, 6, 7, 8, 9, 10, 11, 12, 13, 14, 15% or more relative α-amylase activity than the relative α-amylase activity of the corresponding wild-type parental α-amylase.

The amino acid sequence of the variant polypeptide is different from the wild-type parental polypeptide, and thus the level of amino acid identity between the variant polypeptide and the wild-type parental polypeptide is necessarily less than 100%. The variant polypeptide has at least 50%, 60%, 70%, 80%, 85%, 90%, 91%, 92%, 93%, 94%, 95%, 96%, 97%, 98% or 99% (and less than 100%) identity to the amino acid sequence of the wild-type parental archaeal/bacterial α-amylase. The amino acid sequence of the variant polypeptide has at least one added, deleted or substituted amino acid residue when compared to the wild-type parental archaeal/bacterial α-amylase. In an embodiment, the wild-type parental is a bacterial alpha-amylase. In another embodiment, the wild-type parental polypeptide is an archaeal alpha-amylase.

The variant polypeptide also exhibits alpha-amylase activity. In an embodiment, the variant polypeptide exhibits more α-amylase activity and/or relative-amylase activity when compared to the wild-type parental archaeal/bacterial α-amylase when determined at elevated temperatures (such as, for example, a temperature of about 60° C., 61° C., 62° C., 63° C., 64° C. 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or more).

In an embodiment, the variant polypeptide includes at least one, and in some embodiments, a plurality of amino acid substitutions. An amino acid substitution refers to the replacement of one amino acid residue with another amino acid residue (when compared to the corresponding wild-type parental alpha-amylase). In some embodiments, the amino acid substitution is not a conservative one (e.g., the substituted amino acid residue has different biochemical properties than the replacing amino acid residue).

Alternatively or in combination, the variant polypeptide includes at least one, and in some embodiments a plurality, of amino acid additions. An amino acid addition refers to the inclusion of at least one amino acid residue to the corresponding wild-type parental alpha-amylase. Even though additions can be made throughout the amino acid sequence of the wild-type parental alpha-amylases, in some embodiments, the variant polypeptide can include one or more added amino acid residues at the N-terminus, and/or at the C-terminus of the wild-type parental alpha-amylase to generate the variant alpha-amylase. In a specific embodiment, the variant polypeptide can include a methionine residue (M), a methionine and serine residues (MS), or a methionine, lysine and tyrosine (MKY) residues at the N-terminus. In such embodiments, the initial methionine residue (M), methionine and serine residues (MS), or methionine, lysine and tyrosine (MKY) residues at the N-terminus can be cleaved from the pre-protein to provide the mature protein.

Alternatively or in combination, the improved polypeptide includes at least one, and in some embodiments a plurality of amino acid deletions. An amino acid deletion refers to the removal of at least one amino acid residues when compared to the corresponding wild-type alpha-amylase. The at least one amino acid deletion can be located at the N-terminus, the C-terminus or both at the N- and C-terminus. Alternatively or in combination, the at least one amino acid can be located inside the amino acid sequence of the variant polypeptide.

In an embodiment, the variant polypeptide having improved biological properties is obtained from modifying the amino acid sequence of a wild-type parental archaeal alpha-amylase. For example, the polypeptide having improved biological properties can be derived from a Thermococcus archaeal alpha-amylase, and in specific embodiments, from a Thermococcus hydrothermalis archaeal alpha-amylase. In a specific embodiments, the variant polypeptide having improved biological properties is obtained/derived from the amino acid sequence of

SEQ ID NO: 13. In such embodiments, the variant polypeptide having improved biological properties exhibits less dependence on the presence of a metallic ion, higher thermostability and/or higher resistance to chelation than the polypeptide having/consisting of the amino acid sequence of SEQ ID NO: 13. In addition, the variant polypeptide having improved biological properties has at least 70% identity the amino acid sequence of SEQ ID NO: 13, and less than 100% identity with respect to the amino acid sequence of SEQ ID NO: 13.

The polypeptide having improved properties can have, in some embodiments, an amino acid residue corresponding to position 116 of the amino acid sequence of SEQ ID NO: 13, which is different from a tyrosine residue (e.g., W116X₀, wherein X₀ is a natural amino acid residue which is not a tryptophan residue). For example, the amino acid residue corresponding to position 116 of the amino acid sequence of SEQ ID NO: 13, can be alanine (W116A), arginine (W116R), asparagine (W116N), aspartic acid (W116D), cysteine (W116C), glutamine (W116Q), glutamic acid (W116E), glycine (W116G), histidine (W116H), isoleucine (W1161). leucine (W116L), lysine (W116K), methionine (W116M), phenylalanine (W116F), proline (W116P), serine (W116S), threonine (W116T), tyrosine (W116Y), or valine (W116V). In another example, the amino acid residue corresponding to position 116 of the amino acid sequence of SEQ ID NO: 13, can be arginine (W116R), glutamic acid (W116E), lysine (W116K), or threonine (W116T).

The polypeptide having improved properties can have, in some embodiments, an amino acid residue corresponding to position 123 of the amino acid sequence of SEQ ID NO: 13, which is different from a tyrosine residue (e.g., Y123X₁, wherein X₁ is a natural amino acid residue which is not a tyrosine residue). For example, the amino acid residue corresponding to position 123 of the amino acid sequence of SEQ ID NO: 13, can be alanine (Y123A), arginine (Y123R), asparagine (Y123N), aspartic acid (Y123D), cysteine (Y123C), glutamine (Y123Q), glutamic acid (Y123E), glycine (Y123G), histidine (Y123H), isoleucine (Y1231), leucine (Y123L), lysine (Y123K), methionine (Y123M), phenylalanine (Y123F), proline (Y123P), serine (Y123S), threonine (Y123T), tryptophan (Y123W), or valine (Y123V). For example, the amino acid residue corresponding to position 123 of the amino acid sequence of SEQ ID NO: 13, can be asparagine (Y123N), aspartic acid (Y123D), glutamic acid (Y123E), or lysine (Y123K). In some embodiments, the amino acid residue corresponding position 123 of the amino acid sequence of SEQ ID NO: 13, can be asparagine (Y123N).

The variant polypeptide having improved properties can have, in some embodiments, an amino acid residue corresponding to position 385 of the amino acid sequence of SEQ ID NO: 13, which is different from a cysteine residue (e.g., C385X₂, wherein X₂ is a natural amino acid residue which is not a cysteine residue). In some embodiments, the amino acid residue corresponding to position 385 of the amino acid sequence of SEQ ID NO: 13 can be alanine (C385A), arginine (C385R), asparagine (C385N), aspartic acid (C385D), glutamine (C385Q), glutamic acid (C385E), glycine (C385G), histidine (C385H), isoleucine (C3851), leucine (C385L), lysine (C385K), methionine (C385M), phenylalanine (C385F), proline (C385P), serine (C385S), threonine (C385T), tryptophan (C385W), tyrosine (C385Y), or valine (C385V). In some embodiments, the amino acid residue corresponding to position 385 of the amino acid sequence of SEQ ID NO: 13 can be arginine (C385R), glutamine (C385Q), glutamic acid (C385E), lysine (C385K), threonine (C385T), or valine (C385V). In some embodiments, the amino acid residue corresponding position 385 of the amino acid sequence of SEQ ID NO: 13 can be glutamine (C385Q).

The variant polypeptide having improved properties can have, in some embodiments, an amino acid residue corresponding to position 429 of the amino acid sequence of SEQ ID NO: 13, which is different from a cysteine residue (e.g., C429X₃, wherein X₃ is a natural amino acid residue which is not a cysteine residue). In some embodiments, the amino acid residue corresponding to position 429 of the amino acid sequence of SEQ ID NO: 13 can be alanine (C429A), arginine (C429R), asparagine (C429N), aspartic acid (C429D), glutamine (C429Q), glutamic acid (C429E), glycine (C429G), histidine (C429H), isoleucine (C4291), leucine (C429L), lysine (C429K), methionine (C429M), phenylalanine (C429F), proline (C429P), serine (C429S), threonine (C429T), tryptophan (C429W), tyrosine (C429Y), or valine (C429V). In some embodiments, the amino acid residue corresponding to position 429 of the amino acid sequence of SEQ ID NO: 13 can be alanine (C429A), proline (C429P), threonine (C429T), or valine (C429V). In some embodiments, the amino acid residue corresponding to position 429 of the amino acid sequence of SEQ ID NO: 13 can be arginine (C429R), asparagine (C429N), aspartic acid (C429D), glutamine (C429Q), glutamic acid (C429E), glycine (C429G), histidine (C429H), isoleucine (C4291), leucine (C429L), lysine (C429K), methionine (C429M), phenylalanine (C429F), serine (C429S), tryptophan (C429W), or tyrosine (C429Y). In some embodiments, the amino acid residue corresponding position 429 of the amino acid sequence of SEQ ID NO: 13 can be valine (C429V).

In some embodiments, the variant polypeptide having improved properties can have, with respect to the amino acid sequence of SEQ ID NO: 13, a single amino acid substitution at one of the following position 123 (e.g., Y123X₁, wherein X₁ is a natural amino acid residue which is not a tyrosine residue), 385 (e.g., C385X₂, wherein X₂ is a natural amino acid residue which is not a cysteine residue) and 429 (e.g., C429X₃, wherein X₃ is a natural amino acid residue which is not a cysteine residue). In specific embodiments, the variant polypeptide having improved properties can have, with respect to the amino acid sequence of SEQ ID NO: 13, a single amino acid substitution at one of the following position 123 (e.g., Y123N), 385 (e.g., C385Q) and 429 (e.g., C429V).

In some embodiments, the variant polypeptide having improved properties can have, with respect to the amino acid sequence of SEQ ID NO: 13, two amino acid substitutions at positions 123 (e.g., Y123X₁, wherein X₁ is a natural amino acid residue which is not a tyrosine residue), 385 (e.g., C385X₂, wherein X₂ is a natural amino acid residue which is not a cysteine residue) and/or 429 (e.g., C429X₃, wherein X₃ is a natural amino acid residue which is not a cysteine residue). For example, the variant polypeptide having improved properties can have. with respect to the amino acid sequence of SEQ ID NO: 13, an amino acid substitutions at positions 123 and 385. In another example, the variant polypeptide having improved properties can have, with respect to the amino acid sequence of SEQ ID NO: 13, an amino acid substitutions at positions 123 and 429. In still another example, the variant polypeptide having improved properties can have, with respect to the amino acid sequence of SEQ ID NO: 13, an amino acid substitutions at positions 385 and 429. In such embodiment, the variant polypeptide can have the amino acid sequence of SEQ ID NO: 56 or 57. In specific embodiments, the polypeptide having improved properties can have, with respect to the amino acid sequence of SEQ ID NO: 13, at least two amino acid substitution at the following position 123 (e.g., Y123N), 385 (e.g., C385Q) and 429 (e.g., C429V). For example, the polypeptide having improved properties can have, with respect to the amino acid sequence of SEQ ID NO: 13, amino acid substitutions at positions 123 (e.g., Y123N), and 385 (e.g., C385Q). In still another example, the polypeptide having improved properties can have, with respect to the amino acid sequence of SEQ ID NO: 13, two amino acid substitutions at the positions 123 (e.g., Y123N), and 429 (e.g., C429V). In yet another example, the polypeptide having improved properties can have, with respect to the amino acid sequence of SEQ ID NO: 13, two amino acid substitutions at positions 385 (e.g., C385Q) and 429 (e.g., C429V). In such embodiment, the variant polypeptide can have the amino acid sequence of SEQ ID NO: 56 or 57.

In some embodiments, the variant polypeptide having improved properties can have, with respect to the amino acid sequence of SEQ ID NO: 13, an amino acid substitution at position 123 (e.g., Y123X₁, wherein X₁ is a natural amino acid residue which is not a tyrosine residue). optionally in combination with a further amino acid substitution at position 385 (e.g., C385X₂, wherein X₂ is a natural amino acid residue which is not a cysteine residue) and/or 429 (e.g., C429X₃, wherein X₃ is a natural amino acid residue which is not a cysteine residue). In specific embodiments, the variant polypeptide having improved properties can have, with respect to the amino acid sequence of SEQ ID NO: 13, an amino acid substitution at position 123 (e.g., Y123N), optionally in combination with another amino acid substitution at positions 385 (e.g., C385Q) and/or 429 (e.g., C429V). For example, the variant polypeptide having improved properties can have, with respect to the amino acid sequence of SEQ ID NO: 13, an amino acid substitution at position 123 (e.g., Y123N), in combination with another amino acid substitution at position 385 (e.g., C385Q). In another example, the variant polypeptide having improved properties can have, with respect to the amino acid sequence of SEQ ID NO: 13, an amino acid substitution at position 123 (e.g., Y123N), in combination with another amino acid substitution at position 429 (e.g., C429V).

In some embodiments, the variant polypeptide having improved properties can have, with respect to the amino acid sequence of SEQ ID NO: 13, three amino acid substitutions at positions 123 (e.g., Y123X₁, wherein X₁ is a natural amino acid residue which is not a tyrosine residue), 385 (e.g., C385X₂, wherein X₂ is a natural amino acid residue which is not a cysteine residue) and 429 (e.g C429X₃, wherein X₃ is a natural amino acid residue which is not a cysteine residue). In such specific embodiment, the variant polypeptide can have the amino acid sequence of SEQ ID NO: 58 or 59. In specific embodiment, the polypeptide having improved properties can have, with respect to the amino acid sequence of SEQ ID NO: 13, amino acid substitutions at positions 123 (e.g., Y123N), 385 (e.g., C385Q) and 429 (e.g., C429V)). In such specific embodiment, the variant polypeptide can have the amino acid sequence of SEQ ID NO: 58 or 59.

The variant polypeptide, derived from the wild-type parental archaeal alpha-amylase of SEQ ID NO: 13, can include one or more added amino acid residues at its N-terminus. In a specific embodiment, the variant polypeptide can include a methionine residue (M), a methionine and serine residues (MS), or a methionine, lysine and tyrosine (MKY) residues at its N-terminus.

In an embodiment, the variant polypeptide having improved biological properties is obtained/derived from modifying the amino acid sequence of a wild-type parental bacterial alpha-amylase. For example, the variant polypeptide having improved biological properties can be derived from a Geobacillus bacterial alpha-amylase, and in specific embodiments, from a Geobacillus stearothermophilus bacterial alpha-amylase. In a specific embodiment, the variant polypeptide having improved biological properties is obtained/derived from the amino acid sequence of SEQ ID NO: 39. In such embodiments, the variant polypeptide having improved biological properties exhibits less dependence on the presence of a metallic ion, higher thermostability and/or higher resistance to chelation than the polypeptide having/consisting of the amino acid sequence of SEQ ID NO: 39. In addition, the variant polypeptide having improved biological properties has at least 70% identity the amino acid sequence of SEQ ID NO: 39, and less than 100% identity with respect to the amino acid sequence of SEQ ID NO: 39.

The variant polypeptide having improved properties can have, in some embodiments, at least one deletion at position 181 (ΔI181) or at position 182 (ΔG182) of the amino acid sequence of SEQ ID NO: 39. In some embodiments, the variant polypeptide having improved properties can have a deletion at position 181 (ΔI181) of the amino acid sequence of SEQ ID NO: 39. In some embodiments, the variant polypeptide having improved properties can have a deletion at position 182 (ΔG182) of the amino acid sequence of SEQ ID NO: 39. In some further embodiments, the variant polypeptide having improved properties can have deletions at positions 181 (ΔI181) and 182 (ΔG182) of the amino acid sequence of SEQ ID NO: 39.

The variant polypeptide having improved properties can have, in some embodiments, an amino acid residue corresponding to position 157 of the amino acid sequence of SEQ ID NO: 39, which is different from an arginine residue (e.g., R157X₄, wherein X₄ is a natural amino acid residue which is not an arginine residue). For example, the amino acid residue corresponding to position 157 of the amino acid sequence of SEQ ID NO: 39, can be alanine (R157A), asparagine (R157N), aspartic acid (R157D), cysteine (R157C), glutamine (R157Q), glutamic acid (R157E), glycine (R157G), histidine (R157H), isoleucine (R1571), leucine (R157L), lysine (R157K), methionine (R157M), phenylalanine (R157F), proline (R157P), serine (R157S), threonine (R157T), tryptophan (R157W), tyrosine (R157Y), or valine (R157V). For example, the amino acid residue corresponding to position 157 of the amino acid sequence of SEQ ID NO: 39, can be asparagine (R157N), aspartic acid (R157D), histidine (R157H), or tyrosine (R157Y). In some embodiments, the amino acid residue corresponding to position 157 of the amino acid sequence of SEQ ID NO: 39, can be tyrosine (R157Y).

The variant polypeptide having improved properties can have, in some embodiments, an amino acid residue corresponding to position 173 of the amino acid sequence of SEQ ID NO: 39, which is different from a serine residue (e.g., S173X₅, wherein X₅ is a natural amino acid residue which is not a serine residue). For example, the amino acid residue corresponding to position 173 of the amino acid sequence of SEQ ID NO: 39, can be alanine (S173A), arginine (S173R), asparagine (S173N), aspartic acid (S173D), cysteine (S173C), glutamine (S173Q), glutamic acid (S173E), glycine (S173G), histidine (S173H), isoleucine (S1731), leucine (S173L), lysine (S173K), methionine (S173M), phenylalanine (S173F), proline (S173P), threonine (S173T), tryptophan (S173W), tyrosine (S173Y), or valine (S173V). For example, the amino acid residue corresponding to position 173 of the amino acid sequence of SEQ ID NO: 39, can be arginine (S173R), asparagine (S173N), glycine (S173G), lysine (S173K). proline (S173P), or threonine (S173T). In some embodiments, the amino acid residue corresponding to position 173 of the amino acid sequence of SEQ ID NO: 39, can be lysine (S173K).

The variant polypeptide having improved properties can have, in some embodiments, an amino acid residue corresponding to position 184 of the amino acid sequence of SEQ ID NO: 39, which is different from an alanine residue (e.g., A184X₆, wherein X₆ is a natural amino acid residue which is not an alanine residue). For example, the amino acid residue corresponding to position 184 of the amino acid sequence of SEQ ID NO: 39, can be arginine (A184R), asparagine (A184N), aspartic acid (A184D), cysteine (A184C), glutamine (A184Q), glutamic acid (A184E), glycine (A184G), histidine (A184H), isoleucine (A1841), leucine (A184L), lysine (A184K), methionine (A184M), phenylalanine (A184F), proline (A184P), serine (A184S), threonine (A184T), tryptophan (A184W), tyrosine (A184Y), or valine (A184V). For example, the amino acid residue corresponding to position 184 of the amino acid sequence of SEQ ID NO: 39, can be aspartic acid (A184D), glutamic acid (A184E), glycine (A184G), serine (A184S), or threonine (A184T). In some embodiments, the amino acid residue corresponding to position 184 of the amino acid sequence of SEQ ID NO: 39, can be threonine (A184T).

The variant polypeptide having improved properties can have, in some embodiments, an amino acid residue corresponding to position 191 of the amino acid sequence of SEQ ID NO: 39, which is different from a threonine residue (e.g., T191X₇, wherein X₇ is a natural amino acid residue which is not a threonine residue). For example, the amino acid residue corresponding to position 191 of the amino acid sequence of SEQ ID NO: 39, can be alanine (T191A), arginine (T191R), asparagine (T191N), aspartic acid (T191D), cysteine (T191C), glutamine (T191Q), glutamic acid (T191E), glycine (T191G), histidine (T191H), isoleucine (T1911), leucine (T191L), lysine (T191K), methionine (T191M), phenylalanine (T191F), proline (T191P), serine (T191S), tryptophan (T191W), tyrosine (T191Y), or valine (T191V). For example, the amino acid residue corresponding to position 191 of the amino acid sequence of SEQ ID NO: 39, can be aspartic acid (T191D), lysine (T191K), or proline (T191P). In some embodiments, the amino acid residue corresponding position 191 of the amino acid sequence of SEQ ID NO: 39, can be proline (T191P).

The variant polypeptide having improved properties can have, in some embodiments, an amino acid residue corresponding to position 193 of the amino acid sequence of SEQ ID NO: 39, which is different from an asparagine residue (e.g., N193X₈, wherein X₈ is a natural amino acid residue which is not an asparagine residue). For example, the amino acid residue corresponding to position 193 of the amino acid sequence of SEQ ID NO: 39, can be alanine (N193A), arginine (N193R), aspartic acid (N193D), cysteine (N193C), glutamine (N193Q), glutamic acid (N193E), glycine (N193G), histidine (N193H), isoleucine (N1931), leucine (N193L), lysine (N193K), methionine (N193M), phenylalanine (N193F), proline (N193P), serine (N193S), threonine (N193T), tryptophan (N193W), tyrosine (N193Y), or valine (N193V). For example, the amino acid residue corresponding to position 193 of the amino acid sequence of SEQ ID NO: 39, can be arginine (N193R), glutamine (N193Q), glutamic acid (N193E), or phenylalanine (N193F). In some embodiments, the amino acid residue corresponding position 193 of the amino acid sequence of SEQ ID NO: 39, can be phenylalanine (N193F).

The variant polypeptide having improved properties can have, in some embodiments, an amino acid residue corresponding to position 242 of the amino acid sequence of SEQ ID NO: 39, which is different from a serine residue (e.g., S242X₉, wherein X₉ is a natural amino acid residue which is not a serine residue). For example, the amino acid residue corresponding to position 242 of the amino acid sequence of SEQ ID NO: 39, can be alanine (S242A), arginine (S242R), asparagine (S242N), aspartic acid (S242D), cysteine (S242C), glutamine (S242Q), glutamic acid (S242E), glycine (S242G), histidine (S242H), isoleucine (S2421), leucine (S242L), lysine (S242K), methionine (S242M), phenylalanine (S242F), proline (S242P), threonine (S242T), tryptophan (S242W), tyrosine (S242Y), or valine (S242V). For example, the amino acid residue corresponding to position 242 of the amino acid sequence of SEQ ID NO: 39, can be alanine (S242A), aspartic acid (S242D), glutamic acid (S242E), or proline (S242P). In some embodiments, the amino acid residue corresponding position 242 of the amino acid sequence of SEQ ID NO: 39, can be alanine (S242A).

The variant polypeptide having improved properties can have, in some embodiments, an amino acid residue corresponding to position 245 of the amino acid sequence of SEQ ID NO: 39, which is different from a proline residue (e.g., P245X₁₀, wherein X₁₀ is a natural amino acid residue which is not a proline residue). For example, the amino acid residue corresponding to position 245 of the amino acid sequence of SEQ ID NO: 39, can be alanine (P245A), arginine (P245R), asparagine (P245N), aspartic acid (P245D), cysteine (P245C), glutamine (P245Q), glutamic acid (P245E), glycine (P245G), histidine (P245H), isoleucine (P2451), leucine (P245L), lysine (P245K), methionine (P245M), phenylalanine (P245F), serine (P245S), threonine (P245T), tryptophan (P245W), tyrosine (P245Y), or valine (P245V).

For example, the amino acid residue corresponding to position 245 of the amino acid sequence of SEQ ID NO: 39, can arginine (P245R), asparagine (P245N), histidine (P245H), or lysine (P245K). In some embodiments, the amino acid residue corresponding position 245 of the amino acid sequence of SEQ ID NO: 39, can be arginine (P245R).

The variant polypeptide having improved properties can have, in some embodiments, an amino acid residue corresponding to position 281 of the amino acid sequence of SEQ ID NO: 39, which is different from an aspartic acid residue (e.g., D281X₁₁, wherein X₁₁ is a natural amino acid residue which is not an aspartic acid residue). For example, the amino acid residue corresponding to position 281 of the amino acid sequence of SEQ ID NO: 39, can be alanine (D281A), arginine (D281R), asparagine (D281N), cysteine (D281C), glutamine (D281Q), glutamic acid (D281E), glycine (D281G), histidine (D281H), isoleucine (D2811), leucine (D281L), lysine (D281K), methionine (D281M), phenylalanine (D281F), proline (D281P), serine (D281S), threonine (D281T), tryptophan (D281W), tyrosine (D281Y), or valine (D281V). For example, the amino acid residue corresponding to position 281 of the amino acid sequence of SEQ ID NO: 39, can be asparagine (D281N), glutamic acid (D281E), or glycine (D281G). In some embodiments, the amino acid residue corresponding position 281 of the amino acid sequence of SEQ ID NO: 39, can be asparagine (D281N).

In some embodiments, the variant polypeptide having improved properties can have a deletion(s) at position(s) 181 and/or 182 (of the amino acid sequence of SEQ ID NO: 39) in combination with at least one substitution at the following position in the amino acid sequence of SEQ ID NO: 39: 157, 173, 184, 191, 193, 242, 245, or 281. In an example, the variant polypeptide having improved properties can have a deletion(s) at position(s) 181 and/or 182 (of the amino acid sequence of SEQ ID NO: 39) in combination with at least two substitutions at the following positions in the amino acid sequence of SEQ ID NO: 39: 157, 173, 184, 191, 193, 242, 245, or 281. In another example, the variant polypeptide having improved properties can have a deletion(s) at position(s) 181 and/or 182 (of the amino acid sequence of SEQ ID NO: 39) in combination with at least three substitutions at the following positions in the amino acid sequence of SEQ ID NO: 39: 157, 173, 184, 191, 193, 242, 245, or 281. In a further example, the variant polypeptide having improved properties can have a deletion(s) at position(s) 181 and/or 182 (of the amino acid sequence of SEQ ID NO: 39) in combination with at least four substitutions at the following positions in the amino acid sequence of SEQ ID NO: 39:157, 173, 184, 191, 193, 242, 245, or 281. In yet another example, the variant polypeptide having improved properties can have a deletion(s) at position(s) 181 and/or 182 (of the amino acid sequence of SEQ ID NO: 39) in combination with at least five substitutions at the following positions in the amino acid sequence of SEQ ID NO: 39: 157, 173, 184, 191, 193, 242, 245, or 281. In still another example, the variant polypeptide having improved properties can have a deletion(s) at position(s) 181 and/or 182 (of the amino acid sequence of SEQ ID NO: 39) in combination with at least six substitutions at the following positions in the amino acid sequence of SEQ ID NO: 39: 157, 173, 184, 191, 193, 242, 245, or 281. In yet a further example, the variant polypeptide having improved properties can have a deletion(s) at position(s) 181 and/or 182 (of the amino acid sequence of SEQ ID NO: 39) in combination with at least seven substitutions at the following positions in the amino acid sequence of SEQ ID NO: 39: 157, 173, 184, 191, 193, 242, 245, or 281. In still a further example, the variant polypeptide having improved properties can have a deletion(s) at position(s) 181 and/or 182 (of the amino acid sequence of SEQ ID NO: 39) in combination with substitutions at the following positions in the amino acid sequence of SEQ ID NO: 39: 157, 173, 184, 191, 193, 242, 245, and 281.

In some embodiments, the variant polypeptide having improved properties can have a deletion at position 181 (of the amino acid sequence of SEQ ID NO: 39) in combination with at least one substitution at the following position in the amino acid sequence of SEQ ID NO: 39: 157, 173, 184, 191, 193, 242, 245, or 281. In an example, the variant polypeptide having improved properties can have a deletion at position 181 (of the amino acid sequence of SEQ ID NO: 39) in combination with at least two substitutions at the following positions in the amino acid sequence of SEQ ID NO: 39: 157, 173, 184, 191, 193, 242, 245, or 281. In another example, the variant polypeptide having improved properties can have a deletion at position 181 (of the amino acid sequence of SEQ ID NO: 39) in combination with at least three substitutions at the following positions in the amino acid sequence of SEQ ID NO: 39: 157, 173, 184, 191, 193, 242, 245, or 281. In a further example, the variant polypeptide having improved properties can have a deletion at position 181 (of the amino acid sequence of SEQ ID NO: 39) in combination with at least four substitutions at the following positions in the amino acid sequence of SEQ ID NO: 39: 157, 173, 184, 191, 193, 242, 245, or 281. In yet another example, the variant polypeptide having improved properties can have a deletion at position 181 (of the amino acid sequence of SEQ ID NO: 39) in combination with at least five substitutions at the following positions in the amino acid sequence of SEQ ID NO: 39: 157, 173, 184, 191, 193, 242, 245, or 281. In still another example, the variant polypeptide having improved properties can have a deletion at position 181 (of the amino acid sequence of SEQ ID NO: 39) in combination with at least six substitutions at the following positions in the amino acid sequence of SEQ ID NO: 39: 157, 173, 184, 191, 193, 242, 245, or 281. In yet a further example, the variant polypeptide having improved properties can have a deletion at position 181 (of the amino acid sequence of SEQ ID NO: 39) in combination with at least seven substitutions at the following positions in the amino acid sequence of SEQ ID NO: 39: 157, 173, 184, 191, 193, 242, 245, or 281. In still a further example, the variant polypeptide having improved properties can have a deletion at position 181 (of the amino acid sequence of SEQ ID NO: 39) in combination with substitutions at the following positions in the amino acid sequence of SEQ ID NO: 39: 157, 173, 184, 191, 193, 242, 245, and 281.

In some embodiments, the variant polypeptide having improved properties can have a deletion at position 182 (of the amino acid sequence of SEQ ID NO: 39) in combination with at least one substitution at the following position in the amino acid sequence of SEQ ID NO: 39: 157, 173, 184, 191, 193, 242, 245, or 281. In an example, the variant polypeptide having improved properties can have a deletion at position 182 (of the amino acid sequence of SEQ ID NO: 39) in combination with at least two substitutions at the following positions in the amino acid sequence of SEQ ID NO: 39: 157, 173, 184, 191, 193, 242, 245, or 281. In another example, the variant polypeptide having improved properties can have a deletion at position 182 (of the amino acid sequence of SEQ ID NO: 39) in combination with at least three substitutions at the following positions in the amino acid sequence of SEQ ID NO: 39: 157, 173, 184, 191, 193, 242, 245, or 281. In a further example, the variant polypeptide having improved properties can have a deletion at position 182 (of the amino acid sequence of SEQ ID NO: 39) in combination with at least four substitutions at the following positions in the amino acid sequence of SEQ ID NO: 39: 157, 173, 184, 191, 193, 242, 245, or 281. In yet another example, the variant polypeptide having improved properties can have a deletion at position 182 (of the amino acid sequence of SEQ ID NO: 39) in combination with at least five substitutions at the following positions in the amino acid sequence of SEQ ID NO: 39: 157, 173, 184, 191, 193, 242, 245, or 281. In still another example, the variant polypeptide having improved properties can have a deletion at position 182 (of the amino acid sequence of SEQ ID NO: 39) in combination with at least six substitutions at the following positions in the amino acid sequence of SEQ ID NO: 39: 157, 173, 184, 191, 193, 242, 245, or 281. In yet a further example, the variant polypeptide having improved properties can have a deletion at position 182 (of the amino acid sequence of SEQ ID NO: 39) in combination with at least seven substitutions at the following positions in the amino acid sequence of SEQ ID NO: 39: 157, 173, 184, 191, 193, 242, 245, or 281. In still a further example, the variant polypeptide having improved properties can have a deletion at position 182 (of the amino acid sequence of SEQ ID NO: 39) in combination with substitutions at the following positions in the amino acid sequence of SEQ ID NO: 39: 157, 173, 184, 191, 193, 242, 245, and 281.

In some embodiments, the variant polypeptide having improved properties can have deletions at positions 181 and 182 (of the amino acid sequence of SEQ ID NO: 39) in combination with at least one substitution at the following position in the amino acid sequence of SEQ ID NO: 39:157, 173, 184, 191, 193, 242, 245, or 281. In an example, the variant polypeptide having improved properties can have deletions at positions 181 and 182 (of the amino acid sequence of SEQ ID NO: 39) in combination with at least two substitutions at the following positions in the amino acid sequence of SEQ ID NO: 39: 157, 173, 184, 191, 193, 242, 245, or 281. In another example, the variant polypeptide having improved properties can have deletions at positions 181 and 182 (of the amino acid sequence of SEQ ID NO: 39) in combination with at least three substitutions at the following positions in the amino acid sequence of SEQ ID NO: 39: 157, 173, 184, 191, 193, 242, 245, or 281. In a further example, the variant polypeptide having improved properties can have deletions at positions 181 and 182 (of the amino acid sequence of SEQ ID NO: 39) in combination with at least four substitutions at the following positions in the amino acid sequence of SEQ ID NO: 39: 157, 173, 184, 191, 193, 242, 245, or 281. In yet another example, the variant polypeptide having improved properties can have deletions at positions 181 and 182 (of the amino acid sequence of SEQ ID NO: 39) in combination with at least five substitutions at the following positions in the amino acid sequence of SEQ ID NO: 39: 157, 173, 184, 191, 193, 242, 245, or 281. In still another example, the variant polypeptide having improved properties can have deletions at positions 181 and 182 (of the amino acid sequence of SEQ ID NO: 39) in combination with at least six substitutions at the following positions in the amino acid sequence of SEQ ID NO: 39: 157, 173, 184, 191, 193, 242, 245, or 281. In yet a further example, the variant polypeptide having improved properties can have deletions at positions 181 and 182 (of the amino acid sequence of SEQ ID NO: 39) in combination with at least seven substitutions at the following positions in the amino acid sequence of SEQ ID NO: 39: 157, 173, 184, 191, 193, 242, 245, or 281. In still a further example, the variant polypeptide having improved properties can have deletions at positions 181 and 182 (of the amino acid sequence of SEQ ID NO: 39) in combination with substitutions at the following positions in the amino acid sequence of SEQ ID NO: 39: 157, 173, 184, 191, 193, 242, 245, and 281.

In embodiments in which the variant polypeptide having improved properties has deletions at positions 181 and 182 (of the amino acid sequence of SEQ ID NO: 39), it can also have an amino acid residue corresponding to position 193 of the amino acid sequence of SEQ ID NO: 39, which is different from an asparagine residue (e.g., N193X₈, wherein X₈ is a natural amino acid residue which is not an asparagine residue). In such embodiment, the amino acid residue corresponding to position 193 of the amino acid sequence of SEQ ID NO: 39, can be alanine (N193A), aspartic acid (N193D), cysteine (N193C), glutamine (N193Q), glutamic acid (N193E), glycine (N193G), histidine (N193H), isoleucine (N1931), leucine (N193L), lysine (N193K), methionine (N193M), phenylalanine (N193F), proline (N193P), serine (N193S), threonine (N193T), tryptophan (N193W), tyrosine (N193Y), or valine (N193V). In addition, the amino acid residue corresponding position 193 of the amino acid sequence of SEQ ID NO: 39, can be phenylalanine (N193F). In a specific embodiment, the polypeptide having improved properties can have a deletion at position 181, a deletion at position 182 as well as an amino acid substitution at position 193 (e.g., N193X₈, which can be, in some further embodiments, N193F). In such embodiments, the variant polypeptide having improved properties can have the amino acid sequence of SEQ ID NO: 45, 46, 47, 48, 49, 50, or 51.

In embodiments in which the variant polypeptide having improved properties has deletions at positions 181 and 182 (of the amino acid sequence of SEQ ID NO: 39) as well as a substitution at position 193 (e.g., N193X₈), it can also have an amino acid residue corresponding to position 242 of the amino acid sequence of SEQ ID NO: 39, which is different from a serine residue (e.g., S242X₉, wherein X₉ is a natural amino acid residue which is not a serine residue). For example, the amino acid residue corresponding to position 242 of the amino acid sequence of SEQ ID NO: 39, can be alanine (S242A), arginine (S242R), asparagine (S242N), aspartic acid (S242D), cysteine (S242C), glutamine (S242Q), glutamic acid (S242E), glycine (S242G), histidine (S242H), isoleucine (S2421), leucine (S242L), lysine (S242K), methionine (S242M), phenylalanine (S242F), proline (S242P), threonine (S242T), tryptophan (S242W), tyrosine (S242Y), or valine (S242V). In some embodiments, the amino acid residue corresponding position 242 of the amino acid sequence of SEQ ID NO: 39, can be alanine (S242A). In a specific embodiment, the polypeptide having improved properties can have a deletion at position 181, a deletion at position 182 as well as amino acid substitutions at positions 193 (e.g., N193X₈, which can be, in some further embodiments, N193F) and 242 (e.g., S242X₉, which can be, in some further embodiments, S242A). In such embodiments, the variant polypeptide having improved properties can have the amino acid sequence of SEQ ID NO: 46, or 47.

In embodiments in which the variant polypeptide having improved properties has deletions at positions 181 and 182 (of the amino acid sequence of SEQ ID NO: 39) as well as substitutions at positions 193 (e.g., N193X₈), and 242 (e.g., S242X₉), it can also have an amino acid residue corresponding to position 245 of the amino acid sequence of SEQ ID NO: 39, which is different from a proline residue (e.g., P245X₁₀, wherein X₁₀ is a natural amino acid residue which is not a proline residue). For example, the amino acid residue corresponding to position 245 of the amino acid sequence of SEQ ID NO: 39, can be alanine (P245A), arginine (P245R), asparagine (P245N), aspartic acid (P245D), cysteine (P245C), glutamine (P245Q), glutamic acid (P245E), glycine (P245G), histidine (P245H), isoleucine (P2451), leucine (P245L), lysine (P245K), methionine (P245M), phenylalanine (P245F), serine (P245S), threonine (P245T), tryptophan (P245W), tyrosine (P245Y), or valine (P245V). In some embodiments, the amino acid residue corresponding position 245 of the amino acid sequence of SEQ ID NO: 39, can be arginine (P245R). In a specific embodiment, the variant polypeptide having improved properties can have a deletion at position 181, a deletion at position 182 as well as amino acid substitutions at positions 193 (e.g., N193X₈, which can be, in some further embodiments. N193F), 242 (e.g., S242X₉, which can be, in some further embodiments, S242A), and 245 ((e.g., P245X₁₀, which can be, in some embodiment, P245R). In such embodiments, the variant polypeptide having improved properties can have the amino acid sequence of SEQ ID NO: 47.

In embodiments in which the variant polypeptide having improved properties has deletions at positions 181 and 182 (of the amino acid sequence of SEQ ID NO: 39) as well as a substitution at position 193 (e.g., N193X₈), it can also have an amino acid residue corresponding to position 157 of the amino acid sequence of SEQ ID NO: 39, which is different from an arginine residue (e.g., R157X₄, wherein X₄ is a natural amino acid residue which is not an arginine residue) as well as an amino acid residue corresponding to position 184 of the amino acid sequence of SEQ ID NO: 39, which is different from an alanine residue (e.g., A184X₆, wherein X₆ is a natural amino acid residue which is not an alanine residue). For example, the amino acid residue corresponding to position 157 of the amino acid sequence of SEQ ID NO: 39, can be alanine (R157A), asparagine (R157N), aspartic acid (R157D), cysteine (R157C), glutamine (R157Q), glutamic acid (R157E), glycine (R157G), histidine (R157H), isoleucine (R1571), leucine (R157L), lysine (R157K), methionine (R157M), phenylalanine (R157F), proline (R157P), serine (R157S), threonine (R157T), tryptophan (R157W), tyrosine (R157Y), or valine (R157V). In some embodiments, the amino acid residue corresponding to position 157 of the amino acid sequence of SEQ ID NO: 39, can be tyrosine (R157Y). For example, the amino acid residue corresponding to position 184 of the amino acid sequence of SEQ ID NO: 39, can be arginine (A184R), asparagine (A184N), aspartic acid (A184D), cysteine (A184C), glutamine (A184Q), glutamic acid (A184E), glycine (A184G), histidine (A184H), isoleucine (A1841), leucine (A184L), lysine (A184K), methionine (A184M), phenylalanine (A184F), proline (A184P), serine (A184S), threonine (A184T), tryptophan (A184W), tyrosine (A184Y), or valine (A184V). In some embodiments, the amino acid residue corresponding to position 184 of the amino acid sequence of SEQ ID NO: 39, can be threonine (A184T). In a specific embodiment, the variant polypeptide having improved properties can have a deletion at position 181, a deletion at position 182 as well as amino acid substitutions at positions 193 (e.g., N193X₈, which can be, in some further embodiments, N193F), 157 (e.g., R157X₄, which can be, in some further embodiments, R157Y), and 184 (e.g., A184X₆, which can be, in some embodiment, A184T). In such embodiments, the variant polypeptide having improved properties can have the amino acid sequence of SEQ ID NO: 48, 49, 50 or 51.

In embodiments in which the variant polypeptide having improved properties has deletions at positions 181 and 182 (of the amino acid sequence of SEQ ID NO: 39) as well as substitutions at positions 193 (e.g., N193X₈), 157 (e.g., R157X₄), and 184 (e.g., A184X₆), it can also have an amino acid residue corresponding to position 281 of the amino acid sequence of SEQ ID NO: 39, which is different from an aspartic acid residue (e.g., D281X₁₁, wherein X₁₁ is a natural amino acid residue which is not an aspartic acid residue). For example, the amino acid residue corresponding to position 281 of the amino acid sequence of SEQ ID NO: 39, can be alanine (D281A), arginine (D281R), asparagine (D281N), cysteine (D281C), glutamine (D281Q), glutamic acid (D281E), glycine (D281G), histidine (D281H), isoleucine (D2811), leucine (D281L), lysine (D281K), methionine (D281M), phenylalanine (D281F), proline (D281P), serine (D281S), threonine (D281T), tryptophan (D281W), tyrosine (D281Y), or valine (D281V). In some embodiments, the amino acid residue corresponding position 281 of the amino acid sequence of SEQ ID NO: 39, can be asparagine (D281N). In a specific embodiment, the variant polypeptide having improved properties can have a deletion at position 181, a deletion at position 182 as well as amino acid substitutions at positions 193 (e.g., N193X₈, which can be, in some further embodiments. N193F), 157 (e.g., R157X₄, which can be, in some further embodiments, R157Y), 184 (e.g., A184X₆, which can be, in some embodiment, A184T), and 281 (e.g., D281X₁₁, which can be, in some embodiments, D281N). In such embodiments, the variant polypeptide having improved properties can have the amino acid sequence of SEQ ID NO: 51.

In embodiments in which the variant polypeptide having improved properties has deletions at positions 181 and 182 (of the amino acid sequence of SEQ ID NO: 39) as well as substitutions at positions 193 (e.g., N193X₈), 157 (e.g., R157X₄), and 184 (e.g., A184X₆), it can also have an amino acid residue at position 173 of the amino acid sequence of SEQ ID NO: 39, which is different from a serine residue (e.g., S173X₅, wherein X₅ is a natural amino acid residue which is not a serine residue). For example, the amino acid residue corresponding to position 173 of the amino acid sequence of SEQ ID NO: 39, can be alanine (S173A), arginine (S173R), asparagine (S173N), aspartic acid (S173D), cysteine (S173C), glutamine (S173Q), glutamic acid (S173E), glycine (S173G), histidine (S173H), isoleucine (S1731), leucine (S173L), lysine (S173K), methionine (S173M), phenylalanine (S173F), proline (S173P), threonine (S173T), tryptophan (S173W), tyrosine (S173Y), or valine (S173V). In some embodiments, the amino acid residue corresponding to position 173 of the amino acid sequence of SEQ ID NO: 39, can be lysine (S173K). In a specific embodiment, the variant polypeptide having improved properties can have a deletion at position 181, a deletion at position 182 as well as amino acid substitutions at positions 193 (e.g., N193X₈, which can be, in some further embodiments, N193F), 157 (e.g., R157X₄, which can be, in some further embodiments, R157Y), 184 (e.g., A184X₆, which can be, in some embodiment, A184T), 281 (e.g., D281X₁₁, which can be, in some embodiments, D281N), and 173 (e.g., S173X₅, which can be, in some embodiments, S173K). In such embodiments, the variant polypeptide having improved properties can have the amino acid sequence of SEQ ID NO: 49.

In embodiments in which the variant polypeptide having improved properties has deletions at positions 181 and 182 (of the amino acid sequence of SEQ ID NO: 39) as well as substitutions at positions 193 (e.g., N193X₈), 157 (e.g., R157X₄), and 184 (e.g., A184X₆), it can also have an amino acid residue corresponding to position 191 of the amino acid sequence of SEQ ID NO: 39, which is different from a threonine residue (e.g., T191X₇, wherein X₇ is a natural amino acid residue which is not a threonine residue). For example, the amino acid residue corresponding to position 191 of the amino acid sequence of SEQ ID NO: 39, can be alanine (T191A), arginine (T191R), asparagine (T191N), aspartic acid (T191D), cysteine (T191C), glutamine (T191Q), glutamic acid (T191E), glycine (T191G), histidine (T191H), isoleucine (T1911), leucine (T191L), lysine (T191K), methionine (T191M), phenylalanine (T191F), proline (T191P), serine (T191S), tryptophan (T191W), tyrosine (T191Y), or valine (T191V). In some embodiments, the amino acid residue corresponding position 191 of the amino acid sequence of SEQ ID NO: 39, can be proline (T191P). In a specific embodiment, the variant polypeptide having improved properties can have a deletion at position 181, a deletion at position 182 as well as amino acid substitutions at positions 193 (e.g., N193X₈, which can be, in some further embodiments, N193F), 157 (e.g., R157X₄, which can be, in some further embodiments, R157Y), 184 (e.g., A184X₆, which can be, in some embodiment, A184T), 281 (e.g., D281X₁₁, which can be, in some embodiments, D281N), and 191 (e.g., T191X₇, which can be, in some embodiments, T191P). In such embodiments, the variant polypeptide having improved properties can have the amino acid sequence of SEQ ID NO: 50.

The variant polypeptide having improved properties that are derived from the wild-type parental bacterial alpha-amylase having the amino acid sequence of SEQ ID NO: 39 do not have (lack) an alanine residue (A) at position 73, a serine residue(S) at position 217, a methionine residue (M) at position 278, an asparagine residue (N) at position 281, a threonine residue (T) at position 304, a valine residue (V) at position 416, an arginine (R) at position 435, a valine (V) at position 489, a serine(S) at position 490, a tryptophan residue (W) at position 492, a serine residue(S) at position 493 and/or an aspartic acid residue (D) at position 501. In some embodiments, the polypeptide having improved properties that are derived from the wild-type parental bacterial alpha-amylase having the amino acid sequence of SEQ ID NO: 39 do not have (lack) an alanine residue (A) at position 73, a serine residue(S) at position 217, a methionine residue (M) at position 278, an asparagine residue (N) at position 281, a threonine residue (T) at position 304, a valine residue (V) at position 416, an arginine (R) at position 435, a valine (V) at position 489, a serine(S) at position 490, a tryptophan residue (W) at position 492, a serine residue(S) at position 493 and an aspartic acid residue (D) at position 501. In further embodiments, the polypeptide having improved properties that are derived from the wild-type parental bacterial alpha-amylase having the amino acid sequence of SEQ ID NO: 39 can have in some embodiments, a threonine residue (T) at position 73, an asparagine residue (N) at position 217, a threonine residue (T) at position 278, an aspartic acid residue (D) at position 281, an alanine residue (A) at position 304, a glycine residue (G) at position 416, a serine(S) at position 435, a threonine (T) at position 489, an isoleucine (I) at position 490, an arginine residue (R) at position 492, a proline residue (P) at position 493 and/or a glycine residue (G) at position 501. The polypeptide having improved properties that are derived from the wild-type parental bacterial alpha-amylase having the amino acid sequence of SEQ ID NO: 39 can have in some embodiments, a threonine residue (T) at position 73, an asparagine residue (N) at position 217, a threonine residue (T) at position 278, an aspartic acid residue (D) at position 281, an alanine residue (A) at position 304, a glycine residue (G) at position 416, a serine(S) at position 435, a threonine (T) at position 489, an isoleucine (I) at position 490, an arginine residue (R) at position 492, a proline residue (P) at position 493 and/or a glycine residue (G) at position 501. In further embodiments, the polypeptide having improved properties that are derived from the wild-type parental bacterial alpha-amylase having the amino acid sequence of SEQ ID NO: 39 can have in some embodiments, a threonine residue (T) at position 73, an asparagine residue (N) at position 217, a threonine residue (T) at position 278, an aspartic acid residue (D) at position 281, an alanine residue (A) at position 304, a glycine residue (G) at position 416, a serine(S) at position 435, a threonine (T) at position 489, an isoleucine (I) at position 490, an arginine residue (R) at position 492, a proline residue (P) at position 493 and a glycine residue (G) at position 501.

The variant polypeptide, derived from the wild-type parental bacterial alpha-amylase of SEQ ID NO: 39, can include one or more added amino acid residues at its N-terminus. In a specific embodiment, the variant polypeptide can include a methionine residue (M), a methionine and serine residues (MS), or a methionine, lysine and tyrosine (MKY) residues at its N-terminus. Microbial host cell and associated products

The recombinant microbial host cell of the present disclosure are capable of expressing at least one alpha-amylase. In some embodiments, the recombinant microbial host cell of the present disclosure are capable of expressing more than one and, in some embodiments, all of the α-amylases present in the enzyme combination. The recombinant microbial host cell thus includes a heterologous nucleic acid molecule intended to allow the expression of (e.g., encoding) one or more α-amylases. In an embodiment, the recombinant microbial host cell can include more than one heterologous nucleic acid molecules for expressing a plurality of alpha-amylases, for example, the at least one archaeal α-amylase and the at least one bacterial α-amylase of the enzyme combination of the present disclosure. In some specific embodiments, the recombinant microbial host cell can include express two (or more) distinct heterologous enzymes which can be each encode a different α-amylase. Each heterologous nucleic acid molecules can be present in one or more copies in the recombinant microbial host cell's genome. The heterologous nucleic acid molecules can be independently integrated in the recombinant microbial host's cell's chromosome at the same or a different locus and/or provided on an independent replicative element. In order to make the recombinant microbial host cells, heterologous nucleic acid molecules are introduced into the recombinant microbial host cell in order to allow the recombinant expression of the one or more α-amylase of the enzyme combination. The recombinant microbial host cell can be a recombinant yeast host cell, such as, for example, from the genus Saccharomyces sp., from the species Saccharomyces cerevisiae, from the genus Komagataella, or from the species Komagataella phaffii. The recombinant microbial host cell can be a recombinant bacterial host cell, such as, for examples from the genus Bacillus sp., from the species Bacillus subtilis, Bacillus amyloliquefaciens, Bacillus stearothermophilus, or Bacillus licheniformis. The recombinant microbial host cell can be a recombinant fungal host cell such as, for example, from the genus Aspergillus sp., Rhizopus sp., from the species Aspergillus niger, Aspergillus oryzyae, or Rhizopus oryzae.

The present disclosure also provides population of at least two distinct recombinant microbial host cells, each of the subpopulations of the population being capable of expressing a different α-amylase or a different combination of α-amylases. In such embodiment, the population has the ability to express the enzyme combination described herein. For example, the heterologous population can include a first microbial host cell subpopulation capable of expressing at least one archaeal α-amylase and a second subpopulation of microbial host cells capable of expressing at least one bacterial α-amylase. The first and second subpopulations of microbial host cells can be propagated together to express their respective α-amylases and provide the enzyme combination. The first and the second subpopulations of microbial host cells can be propagated separately (to express their respective α-amylases) and then admixed with one another to provide the enzyme combination. The population can include one or more distinct recombinant yeast host cell(s), such as, for example, from the genus Saccharomyces sp. or from the species Saccharomyces cerevisiae and/or from the genus Komagataella or from the species Komagataella phaffii. The population can include one or more distinct recombinant bacterial host cell(s), such as, for examples from the genus Bacillus sp. or from the species Bacillus subtilis, Bacillus amyloliquefaciens, Bacillus stearothermophilus or Bacillus licheniformis. The population can include one or more distinct recombinant fungal host cell(s) such as, for example, from the genus Aspergillus sp. or Rhizopus sp., from the species Aspergillus niger, Aspergillus oryzyae or Rhizopus oryzae.

The heterologous nucleic acid molecules of the present disclosure comprise a coding region for the archaeal/bacterial α-amylase. A DNA or RNA “coding region” is a DNA or RNA molecule (preferably a DNA molecule) which is transcribed and/or translated into a heterologous polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The heterologous nucleic acid molecules described herein can comprise transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are considered control regions.

In some embodiments, the heterologous nucleic acid molecules of the present disclosure include a promoter as well as a coding sequence for the archaeal/bacterial α-amylase (or variant thereof). The heterologous nucleic acid sequence can also include a terminator. In the heterologous nucleic acid molecules of the present disclosure, the promoter and the terminator (when present) are operatively linked to the nucleic acid coding sequence of the archaeal/bacterial α-amylase (or variant thereof), e.g., they control the expression and the termination of expression of the nucleic acid sequence of the archaeal/bacterial α-amylase (or variant thereof). When the archaeal/bacterial α-amylase (or variant thereof) is intended to be exported outside the microbial cell, the heterologous nucleic acid molecules of the present disclosure can also include a nucleic acid sequence coding for a signal sequence, e.g., a short peptide sequence for exporting the heterologous archaeal/bacterial α-amylase outside the host cell. When present, the nucleic acid sequence coding for the signal sequence is directly located upstream and is in frame with the nucleic acid sequence coding for the archaeal/bacterial α-amylase (or variant thereof). Once expressed and during the translocation of the archaeal/bacterial-amylase (or variant thereof) outside the recombinant microbial host cell, the signal sequence is cleaved to generate a mature form of the archaeal/bacterial α-amylase (or variant thereof). When the archaeal/bacterial α-amylase (or variant thereof) is intended to be exported inside the nucleus of the microbial cell, the heterologous nucleic acid molecules of the present disclosure can also include a nucleic acid sequence coding for a nuclear localization sequence, e.g., a short peptide sequence for exporting the heterologous archaeal/bacterial α-amylase inside the nucleus the host cell. When present, the nucleic acid sequence coding for the nuclear localization sequence can be directly located downstream and is in frame with the nucleic acid sequence coding for the archaeal/bacterial α-amylase (or variant thereof).

In the heterologous nucleic acid molecule described herein, the promoter and the nucleic acid molecule coding for the archaeal/bacterial α-amylase (or variant thereof) are operatively linked to one another. In the context of the present disclosure, the expressions “operatively linked” or “operatively associated” refers to fact that the promoter is physically associated to the nucleotide acid molecule coding for the archaeal/bacterial α-amylase (or variant thereof) in a manner that allows, under certain conditions, the expression archaeal/bacterial α-amylase (or variant thereof) from the nucleic acid molecule. In an embodiment, the promoter can be located upstream (5′) of the nucleic acid sequence coding for the archaeal/bacterial α-amylase (or variant thereof). In still another embodiment, the promoter can be located downstream (3′) of the nucleic acid sequence coding for the archaeal/bacterial α-amylase (or variant thereof). In the context of the present disclosure, one or more than one promoter can be included in each of the heterologous nucleic acid molecule. When more than one promoters are included in the heterologous nucleic acid molecule, each of the promoters is operatively linked to the nucleic acid sequence coding for the archaeal/bacterial α-amylase (or variant thereof). The promoters can be located, in view of the nucleic acid molecule coding for the archaeal/bacterial α-amylase (or variant thereof), upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. Promoters which cause a gene to be expressed during the propagation phase of a microbial cell are herein referred to as “propagation promoters”. Propagation promoters include both constitutive and inducible promoters, such as, for example, glucose-regulated, molasses-regulated, stress-response promoters (including osmotic stress response promoters) and aerobic-regulated promoters. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be native or heterologous to the nucleic acid molecule encoding the archaeal/bacterial α-amylase (or variant thereof). The promoter can be heterologous or derived from a strain being from the same genus or species as the recombinant microbial host cell. The promoter can be a single promoter or a combination of different promoters.

One or more promoters can be used to allow the expression of each archaeal/bacterial α-amylase (or variant thereof) in the recombinant microbial host cell. In the context of the present disclosure, the expression “functional fragment of a promoter” when used in combination to a promoter refers to a shorter nucleic acid sequence than the native promoter which retain the ability to control the expression of the nucleic acid sequence encoding the enzyme combination. Usually, functional fragments are either 5′ and/or 3′ truncation of one or more nucleic acid residue from the native promoter nucleic acid sequence.

In some embodiments, the heterologous nucleic acid molecules include one or a combination of terminators to end the translation of the archaeal/bacterial α-amylase (or variant thereof) coding sequence. The terminator can be native or heterologous to the nucleic acid sequence encoding the archaeal/bacterial α-amylase. In some embodiments, one or more terminators can be used. In further embodiments, the terminator can include a functional variant of a terminator. In the context of the present disclosure, the expression “functional variant of a terminator” refers to a nucleic acid sequence that has been substituted in at least one nucleic acid position when compared to the native terminator and nevertheless retains the ability to end the expression of the nucleic acid sequence coding for the archaeal/bacterial α-amylase (or variant thereof). In the context of the present disclosure, the expression “functional fragment of a terminator” refers to a shorter nucleic acid sequence than the native terminator which retains the ability to end the expression of the nucleic acid sequence coding for the archaeal/bacterial α-amylase (or variant thereof).

In some embodiments, the heterologous nucleic acid molecules include a coding sequence for one or a combination of signal sequence(s) allowing the export of the archaeal/bacterial α-amylase (or variant thereof) outside the microbial host cell. The signal sequence can simply be added to the heterologous nucleic acid molecule (usually in frame with the sequence encoding the archaeal/bacterial α-amylase or variant thereof) or replace the signal sequence already present in the wild-type archaeal/bacterial α-amylase. The signal sequence can be native or heterologous to the heterologous nucleic acid sequence encoding the archaeal/bacterial α-amylase (or variant thereof). In some embodiments, one or more signal sequences can be used. It is understood that the amino acid sequence of the signal sequence is cleaved upon the export of the archaeal/bacterial α-amylase (or variant thereof) outside the recombinant microbial cell and is usually absent from the mature archaeal/bacterial α-amylase (or variant thereof).

The heterologous nucleic acid molecule encoding the archaeal/bacterial α-amylase (or variant thereof) can be integrated in the chromosome of the microbial host cell. The term “integrated” as used herein refers to genetic elements that are placed, through molecular biology techniques, into the chromosome of a host cell. For example, genetic elements can be placed into the chromosomes of the host cell as opposed to in a vector such as a plasmid carried by the host cell. Methods for integrating genetic elements into the chromosome of a host cell are well known in the art and include, without limitation, homologous recombination. The heterologous nucleic acid molecule can be present in one or more copies in the microbial host cell's genome. The heterologous nucleic acid molecule can be integrated in one or more copies in the microbial host cell's chromosome. Alternatively, the heterologous nucleic acid molecule can be independently replicating from the microbe's genome. In such embodiment, the nucleic acid molecule can be stable and self-replicating.

In some embodiments, the heterologous nucleic acid molecules which can be introduced into the recombinant host cells are codon-optimized with respect to the intended recipient recombinant microbial host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules can be introduced in the microbial host cell using a vector or an expression cassette. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The archaeal and/or bacterial α-amylase (or variant thereof) can be expressed in the recombinant microbial host cell in a cell-associated form. As used in the context of the present disclosure, the expression “cell-associated” refers to the fact that, once expressed, the archaeal/bacterial α-amylase (or variant thereof) remain physically associated with the recombinant microbial host cells. In an embodiment, the cell-associated α-amylase (or variant thereof) is expressed and remains inside the cell (e.g., intracellular form). In another embodiment, the cell-associated α-amylase (or variant thereof) is expressed and exported, but remains physically associated with the yeast membrane and/or cell wall (e.g., tethered form). In another embodiment, the archaeal and/or bacterial α-amylase (or variant thereof) can be expressed and exported by the recombinant microbial host cell in a free form. As used in the context of the present disclosure, the expression “free form” refers to the fact that, once expressed, the archaeal/bacterial α-amylase is secreted outside the microbial host cells and it is not intended to remain physically associated with the recombinant microbial host cells. In another embodiment, the archaeal/bacterial α-amylase (or variant thereof) of the present disclosure can be exported in a tethered or free form by the recombinant microbial host cell. In such embodiment, a signal sequence can be provided and is cleaved during the export of the enzyme. As such, the signal sequence, when present, does not form part of the mature form of the enzyme. However, it is possible that, in some embodiments even when the archaeal/bacterial α-amylase (or variant thereof) is intended to be translocated outside the recombinant microbial host cell some immature intracellular archaeal/bacterial α-amylase still possess a signal sequence while they are being processed for export.

The recombinant microbial cell can include a further genetic modification (e.g., a further heterologous nucleic acid molecule) encoding a further heterologous enzyme, such as a further lytic enzyme (e.g., an enzyme involved in the cleavage or hydrolysis of its substrate). In still another embodiment, the lytic enzyme can be a glycoside hydrolase. In the context of the present disclosure, the term “glycoside hydrolase” refers to an enzyme involved in carbohydrate digestion, metabolism and/or hydrolysis, including amylases, cellulases, hemicellulases, cellulolytic and amylolytic accessory enzymes, inulinases, levanases, trehalases, pectinases, xylanases, xylosidases, arabinofuranosidases, galactosidases, endoglucanases and/or pentose sugar utilizing enzymes. In another embodiment, the lytic enzyme can be a protease. In the context of the present disclosure, the term “protease” refers to an enzyme involved in protein digestion, metabolism and/or hydrolysis. In yet another embodiment, the enzyme can be an esterase. In the context of the present disclosure, the term “esterase” refers to an enzyme involved in the hydrolysis of an ester from an acid or an alcohol, including phosphatases such as phytases.

As used in the context of the present disclosure, the expression “hydrolase” (E.C. 3) refers to a protein having enzymatic activity and capable of catalyzing the hydrolysis of a chemical bound. For example, the hydrolase can be an esterase (E.C. 3.1 for example phytase, lipase, phospholipase A1 and/or phospholipase A2), can cleaved C—N non-peptide bonds (E.C. 3.5 for example an asparaginase), can be a glycosylase (E.C. 3.2 for example an amylase (E.C. 3.2.1.1), a glucanase, a glycosidase (E.C. 3.2.1), a cellulase (E.C. 3.2.1.4), a trehalase (E.C. 3.2.1.28), a pectinase and/or a lactase (E.C. 3.2.1.108)), a protease (E.C. 3.4 for example a bacterial protease, a plant protease or a fungal protease). When the hydrolase is an amylase, it can be, for example, a fungal alpha amylase, a bacterial alpha amylase, a maltogenic alpha amylase, a maltotetrahydrolase, a plant (e.g., barley) alpha or beta amylase, a fungal alpha amylase and/or a glucoamylase. When the hydrolase is a glycosidase, it can be, for example, a beta glucosidase. When the hydrolase is a cellulase, it can be, for example, a cellulase and/or an hemicellulase (such as, for example, a xylanase, a xylosidase, an arabinofuranosidase, a galactosidase and/or an endoglucanase).

In some embodiments, the hydrolase is an amylolytic enzyme. As used herein, the expression “amylolytic enzyme” refers to a class of enzymes capable of hydrolyzing starch or hydrolyzed starch. Amylolytic enzymes include, but are not limited to α-amylases (EC 3.2.1.1, sometimes referred to fungal α-amylase, see below), maltogenic amylase (EC 3.2.1.133), glucoamylase (EC 3.2.1.3), glucan 1,4-α-maltotetraohydrolase (EC 3.2.1.60), pullulanase (EC 3.2.1.41), iso-amylase (EC 3.2.1.68) and amylomaltase (EC 2.4.1.25). In an embodiment, the one or more amylolytic enzymes can be an alpha-amylase from Aspergillus oryzae, Saccharomycopsis fibuligera (GenBank Accession #CAA29233.1), and Bacillus amyloliquefaciens (GenBank Accession #ABS72727); a maltogenic alpha-amylase from Geobacillus stearothermophilus; a glucan 1,4-alpha-maltotetraohydrolase from Pseudomonas saccharophila; a pullulanase from Bacillus naganoensis: a pullulanase from Bacillus acidopullulyticus; and/or an iso-amylase from Pseudomonas amyloderamosa; amylomaltase from Thermus thermophiles.

In some embodiments, the hydrolase is a trehalase enzyme. As used herein, the expression “trehalase enzyme” refers to a class of enzymes capable of catalyzing the conversion of trehalose to glucose. In an embodiment, the one or more trehalase enzymes can be a trehalase from Aspergillus fumigatus (GenBank Accession #XP_748551) or Neurospora crassa (GenBank Accession #XP_960845.1).

The additional heterologous enzyme can be a “cellulolytic enzyme”, an enzyme involved in cellulose digestion, metabolism and/or hydrolysis. The term “cellulase” refers to a class of enzymes that catalyze cellulolysis (i.e. the hydrolysis of cellulose). Several different kinds of cellulases are known, which differ structurally and mechanistically. There are general types of cellulases based on the type of reaction catalyzed: endocellulase breaks internal bonds to disrupt the crystalline structure of cellulose and expose individual cellulose polysaccharide chains; exocellulase cleaves 2-4 units from the ends of the exposed chains produced by endocellulase, resulting in the tetrasaccharides or disaccharide such as cellobiose. There are two main types of exocellulases (or cellobiohydrolases, abbreviate CBH)—one type working processively from the reducing end, and one type working processively from the non-reducing end of cellulose; cellobiase or beta-glucosidase hydrolyses the exocellulase product into individual monosaccharides; oxidative cellulases that depolymerize cellulose by radical reactions, as for instance cellobiose dehydrogenase (acceptor); cellulose phosphorylases that depolymerize cellulose using phosphates instead of water. In the most familiar case of cellulase activity, the enzyme complex breaks down cellulose to beta-glucose. A “cellulase” can be any enzyme involved in cellulose digestion, metabolism and/or hydrolysis, including an endoglucanase, glucosidase, cellobiohydrolase, xylanase, glucanase, xylosidase, xylan esterase, arabinofuranosidase, galactosidase, cellobiose phosphorylase, cellodextrin phosphorylase, mannanase, mannosidase, xyloglucanase, endoxylanase, glucuronidase, acetylxylanesterase, arabinofuranohydrolase, swollenin, glucuronyl esterase, expansin, pectinase, and feruoyl esterase protein.

The additional heterologous enzyme can have “hemicellulolytic activity”, an enzyme involved in hemicellulose digestion, metabolism and/or hydrolysis. The term “hemicellulase” refers to a class of enzymes that catalyze the hydrolysis of cellulose. Several different kinds of enzymes are known to have hemicellulolytic activity including, but not limited to, xylanases and mannanases.

The additional heterologous enzyme can have “xylanolytic activity”, an enzyme having the is ability to hydrolyze glycosidic linkages in oligopentoses and polypentoses. The term “xylanase” is the name given to a class of enzymes which degrade the linear polysaccharide beta-1,4-xylan into xylose, thus breaking down hemicellulose, one of the major components of plant cell walls. Xylanases include those enzymes that correspond to Enzyme Commission Number 3.2.1.8. The heterologous enzyme can also be a “xylose metabolizing enzyme”, an enzyme involved in xylose digestion, metabolism and/or hydrolysis, including a xylose isomerase, xylulokinase, xylose reductase, xylose dehydrogenase, xylitol dehydrogenase, xylonate dehydratase, xylose transketolase, and a xylose transaldolase protein. A “pentose sugar utilizing enzyme” can be any enzyme involved in pentose sugar digestion, metabolism and/or hydrolysis, including xylanase, arabinase, arabinoxylanase, arabinosidase, arabinofuranosidase, arabinoxylanase, arabinosidase, and arabinofuranosidase, arabinose isomerase, ribulose-5-phosphate 4-epimerase, xylose isomerase, xylulokinase, xylose reductase, xylose dehydrogenase, xylitol dehydrogenase, xylonate dehydratase, xylose transketolase, and/or xylose transaldolase. In an embodiment, the one or more xylanase enzymes can be a xylanase from Aspergillus niger (GenBank Accession #CAA03655.1) (and have, for example, the amino acid sequence of SEQ ID NO: 72, a variant thereof or a fragment thereof).

The additional heterologous enzyme can have “mannanic activity”, an enzyme having the is ability to hydrolyze the terminal, non-reducing 3-D-mannose residues in β-D-mannosides. Mannanases are capable of breaking down hemicellulose, one of the major components of plant cell walls. Xylanases include those enzymes that correspond to Enzyme Commission Number 3.2.25.

The additional heterologous enzyme can be a “pectinase”, an enzyme, such as pectolyase. pectozyme and polygalacturonase, commonly referred to in brewing as pectic enzymes. These enzymes break down pectin, a polysaccharide substrate that is found in the cell walls of plants.

The additional heterologous enzyme can have “phytolytic activity”, an enzyme catalyzing the conversion of phytic acid into inorganic phosphorus. Phytases (EC 3.2.3) can be belong to the histidine acid phosphatases, β-propeller phytases, purple acid phosphastases or protein tyrosine phosphatase-like phytases family. In an embodiment, the one or more phytase enzymes can be a phytase from Citrobacter braakii (GenBank Accession #AY471611.1) (and have, for example, the amino acid sequence of SEQ ID NO: 73, a variant thereof or a fragment thereof).

The additional heterologous enzyme can have “proteolytic activity”, an enzyme involved in protein digestion, metabolism and/or hydrolysis, including serine proteases, threonine proteases, cysteine proteases, aspartate proteases (e.g., proteases having aspartic activity), glutamic acid proteases and metalloproteases. In some embodiments, the heterologous enzyme having proteolytic activity is a protease enzyme. In an embodiment, the one or more protease enzymes can be a protease from Saccharomycopsis fibuligera (GenBank Accession #P22929) or Aspergillus fumigatus (GenBank Accession #P41748).

In an embodiment, the recombinant microbial host cell is a recombinant bacterial host cell. In an embodiment, the recombinant bacterial host cell can be a Gram-negative bacterial cell. For example, the recombinant bacterial host cell can be from the genus Escherichia (such as for example, from the species Escherichia coli) or from the genus Zymomonas (such as, for example, from the species Zymomonas mobilis). In another embodiment, the recombinant bacterial host cell can be a Gram-positive bacterial cell. In yet another embodiment, the recombinant bacterial host cell can be a lactic acid bacteria or LAB. LAB are a group of Gram-positive bacteria, non-respiring non-spore-forming, cocci or rods, which produce lactic acid as the major end product of the fermentation of carbohydrates. Bacterial genus of LAB include, but are not limited to, Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus, and Weissella. Bacterial species of LAB include, but are not limited to, Lactococcus lactis, Lactococcus garviae, Lactococcus raffinolactis, Lactococcus plantarum, Oenococcus oeni, Pediococcus pentosaceus, Pediococcus acidilactici, Carnococcus allantoicus, Camobacterium gallinarum, Vagococcus fessus, Streptococcus thermophilus, Enterococcus phoeniculicola, Enterococcus plantarum, Enterococcus raffinosus, Enterococcus avium, Enterococcus pallens Enterococcus hermanniensis, Enterococcus faecalis, and Enterococcus faecium. In an embodiment, the LAB is a Lactobacillus and, in some additional embodiments, the Lactobacillus species is L. acetotolerans, L. acidifarinae, L. acidipiscis, L. acidophilus, L. agilis, L. algidus. L. alimentarius, L. amylolyticus, L. amylophilus, L. amylotrophicus, L. amylovorus, L. animalis, L. antri, L. apodemi, L. aviarius, L. bifermentans, L. brevis, L. buchneri, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii (including L. delbrueckii subsp. bulgaricus, L. delbrueckii subsp. delbrueckii, L. delbrueckii subsp. lactis), L. dextrinicus, L. diolivorans, L. equi, L. equigenerosi, L. farraginis, L. farciminis, L. fermentum, L. fornicalis, L. fructivorans, L. frumenti, L. fuchuensis, L. gallinarum, L. gasseri, L. gastricus, L. ghanensis, L. graminis, L. ammesii, L. hamsteri, L. harbinensis, L. hayakitensis, L. helveticus, L. hilgardii, L. omohiochii, L. iners, L. ingluviei, L. intestinalis, L. jensenii, L. johnsonii, L. kalixensis, L. efiranofaciens, L. kefiri, L. kimchii, L. kitasatonis, L. kunkeei, L. leichmannii, L. lindneri, L. alefermentans, L. mali, L. manihotivorans, L. mindensis, L. mucosae, L. murinus, L. nagelii, L. namurensis, L. nantensis, L. oligofermentans, L. oris, L. panis, L. pantheris, L. parabrevis, L. parabuchneri, L. paracasei, L. paracollinoides, L. parafarraginis, L. parakefiri, L. aralimentarius, L. paraplantarum, L. pentosus, L. perolens, L. plantarum, L. pontis, L. protectus, L. psittaci, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. ultunensis, L. vaccinostercus, L. vaginalis, L. versmoldensis, L. vini, L. vitulinus, L. zeae or L. zymae. In another embodiment, the recombinant bacterial host cell is a recombinant Bacillus sp. host cell. In specific embodiment, the recombinant bacterial host cell is from the species Bacillus subtilis. Bacillus licheniformis, Bacillus amyloliquefaciens, Bacillus laevolacticus, Bacillus acidicola, Bacillus coagulans, Bacillus cereus, Bacillus lentis, Bacillus clausii or Bacillus brevis.

In an embodiment, the recombinant microbial host cell is a recombinant yeast host cell. The recombinant yeast host cell can be, for example, from the genus Saccharomyces, Kluyveromyces, Arxula, Debaryomyces, Candida, Pichia, Komagataella, Phaffia, Schizosaccharomyces, Hansenula, Kloeckera, Schwanniomyces, Torula or Yarrowia. Suitable yeast species can include, for example, S. cerevisiae, S. bulderi, S. barnetti, S. exiguus, S. uvarum, S. diastaticus, S. boulardii, C. utilis, K. lactis, K. marxianus or K. fragilis. In some embodiments, the yeast is selected from the group consisting of Saccharomyces cerevisiae, Schizzosaccharomyces pombe, Candida albicans, Komagataella phaffii, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In some further embodiments, the yeast is of Saccharomyces cerevisiae, Schizzosaccharomyces pombe, Candida albicans, Komagataella phaffii, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe or Schwanniomyces occidentalis. In one particular embodiment, the yeast is Saccharomyces cerevisiae. In some embodiments, the recombinant yeast host cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiment, the recombinant yeast host cell can be an oleaginous microalgae host cell (e.g., for example, from the genus Thraustochytrium or Schizochytrium). In an embodiment, the recombinant yeast host cell is from the genus Saccharomyces and, in some embodiments, from the species Saccharomyces cerevisiae. In an embodiment, the recombinant yeast host cell is from the genus Komagataella and, in some embodiments, from the species Komagataella phaffii.

The recombinant microbial host cell of the present disclosure (optionally a microbial population comprising same or a product derived therefrom) can be added to a liquefaction medium to provide the variant polypeptide or the enzyme combination to favor the hydrolysis of the starch (e.g., the hydrolysis of the liquefaction medium). As it is known in the art, an untreated or raw liquefaction medium can readily be used by a fermenting yeast to make at least one fermentation product. However, the liquefaction medium is often required to mechanically process the raw liquefaction medium so it can be used in a subsequent fermentation. On the other hand, it is possible to treat the liquefaction medium with a combination of heat and a source of alpha-amylase activity to generate a fermentation medium (an hydrolyzed liquefaction medium in this case). The liquefaction medium is a substrate comprising starch molecules intended to be cleaved into dextrins or smaller carbohydrates, at least in part, prior to fermentation, at least in part by the action of the archaeal/bacterial α-amylase of the enzyme combination. In the art, the liquefaction medium can be referred to as a slurry when the liquefaction medium is derived from corn. As it is known in the art, a slurry is a substrate whose physical integrity has been modified by crushing it. The slurry is usually suspended in water and may optionally be submitted to a heat treatment (prior to, during and/or after it has been contacted by the enzyme combination of the present disclosure).

Alternatively or in combination, the recombinant microbial host cell of the present disclosure (or a microbial population comprising same or a product derived therefrom) can be used to generate a source of alpha-amylase and, in some embodiments, the enzyme combination described herein. In such embodiment, the recombinant microbial host cell/population is placed in a culture medium under a condition so as to allow the expression of the one or more alpha-amylases. In embodiments in which a microbial population is used, the different microbial strains present in the microbial population can be cultured in separate culture media or in the same culture medium under a condition so as to allow the expression of the enzymes of the combination. Once the alpha-amylase(s) has been expressed, the recombinant microbial host cell/microbial population can be used “as is” or can optionally be disrupted or inactivated to provide the enzyme combination with an inactivated form of the recombinant microbial host cell (e.g., an inactivated microbial product). The process can include disrupting the integrity of the recombinant microbial host cell to inactivate, at least in part, the recombinant microbial host cell. In some embodiments, mechanical, thermal, chemical and/or enzymatic means can be used to disrupt the integrity of the recombinant microbial host cell. In some embodiments, the disruption of the integrity of the recombinant microbial host cells can be achieved by using mechanical means such as, for example, homogenization (including high-pressure homogenization) and bead beating.

In some embodiments, the alpha-amylase(s) (or variants thereof) that has been produced by the recombinant microbial host cell (or a microbial population comprising same) can optionally be purified/isolated, at least in part, from the recombinant microbial host cell having expressed it. As used in the context of the present disclosure, the expression “isolating/purifying the archaeal/bacterial alpha amylase(s)” refers to the removal of at least some of the components of the recombinant microbial host cell from the archaeal/bacterial alpha amylase(s) (or variant(s) thereof) and providing same in an isolated/purified form. In some embodiments, the archaeal/bacterial alpha amylase(s) can independently be provided in a substantially isolated/purified form. The expression “substantially isolating/purifying the enzyme combination” refers to the removal of the majority of the components of the recombinant microbial host cell from the archaeal/bacterial alpha amylase(s) (or variant(s) thereof) and providing same in a substantially isolated/purified form. The archaeal/bacterial alpha amylase(s) preparations can thus include a component from the microbial host cell (cell wall, cell membrane, organelle membrane, proteins, DNA, lipids, etc.). The process of providing the archaeal/bacterial alpha amylase(s) preparation can include disrupting the integrity of the recombinant microbial host cell to inactivate, at least in part (and in some embodiments lyse) at least in part, the recombinant microbial host cells. In some embodiments, the process comprises submitting the recombinant microbial host cell to an homogenization step (such as, for example, a high-pressure homogenization step) to provide homogenized microbial host cells. In some embodiments, the process comprises submitting the recombinant microbial host cell to bead beating step to provide bead beaten microbial host cells. The process can include centrifuging and/or filtering the culture medium and/or the recombinant microbial host cell (provided in an active or an inactive form). The process can include drying the archaeal/bacterial alpha-amylase to independently provide them in a solid or dried form.

The present disclosure provides a microbial product, which can be a yeast product, e.g., a product obtained from a recombinant yeast host cell or a population of recombinant yeast host cells having expressed at least one or both of the archaeal/bacterial alpha-amylases (or variants thereof). The yeast product can be an active or semi-active product, such as, for example, a cream yeast or propped yeast cell. The yeast product can be, for example, an inactivated whole cell yeast, a yeast lysate (e.g., an autolysate), a yeast extract, and/or a yeast fraction (e.g., yeast cell walls). The yeast extract can be a bead-milled yeast extract obtained from bead milling the yeast cell. The yeast extract can be a bead-beaten yeast extract obtained from bead beating the yeast cell. The yeast extract can be a high-pressure homogenized yeast extract obtained from high pressure homogenizing the yeast cell. The yeast product can be made prior to the beginning of the liquefaction by means known to those skilled in the art. Alternatively or in combination, the yeast product can be generated in situ prior to fermentation (for example during liquefaction) by adding the recombinant yeast host cell (or populations thereof) to the liquefaction medium and submitting the liquefaction medium to a heat treatment step (as described herein). In some additional embodiments, the enzymatic combination comprises a combination of microbial products, a first microbial product providing the at least one archaeal alpha-amylase (or variant thereof) and a second microbial product providing the at least one bacterial alpha-amylase (or variant thereof). In some further embodiments, the enzymatic combination comprises a combination of yeast products, a first yeast product providing the at least one archaeal alpha-amylase (or variant thereof) and a second yeast product providing the at least one bacterial alpha-amylase (or variant thereof).

In embodiments in which both the at least one archaeal alpha-amylase (or variant thereof) and the at least bacterial alpha-amylase (or variant thereof) are provided in an inactivated form (e.g. inactivated whole cell yeasts for example), the enzymatic combination can include, in weight percent of the final microbial product, at least 5, 10, 15, 20, 25, 30, 35, 40, 45, 50, 55, 60, 65, 70, 75, 80, 85, 90, 95% or more of the at least one archaeal alpha-amylase (or variant thereof). In some embodiments, the enzymatic combination can include, in weight percent of the final microbial product, at least 25% or more of the at least one archaeal alpha-amylase (or variant thereof). In some embodiments, the enzymatic combination can include, in weight percent of the final microbial product, at least 50% or more of the at least one archaeal alpha-amylase (or variant thereof). In some embodiments, the enzymatic combination can include, in weight percent of the final microbial product, at least 75% or more of the at least one archaeal alpha-amylase (or variant thereof). In some additional embodiments, the enzymatic combination can include, in weight percent of the final microbial product, at least 5, 10, 15, 20, 25, 30, 35, 40, 45, 50, 55, 60, 65, 70, 75, 80, 85, 90, 95% or more of the at least one bacterial alpha-amylase (or variant thereof). In some embodiments, the enzymatic combination can include, in weight percent of the final microbial product, at least 25% or more of the at least one bacterial alpha-amylase (or variant thereof). In some embodiments, the enzymatic combination can include, in weight percent of the final microbial product, at least 50% or more of the at least one bacterial alpha-amylase (or variant thereof). In some embodiments, the enzymatic combination can include, in weight percent of the final microbial product, at least 75% or more of the at least one bacterial alpha-amylase (or variant thereof). In some further embodiments, the enzymatic combination can include, in weight percent of the final microbial product, no more than 95, 90, 85, 80, 75, 70, 65, 60, 55, 50, 45, 40, 35, 30, 25, 20, 15, 10, 5% or less of the at least one archaeal alpha-amylase (or variant thereof). In some embodiments, the enzymatic combination can include, in weight percent of the final microbial product, no more than 25% of the at least one archaeal alpha-amylase (or variant thereof). In some embodiments, the enzymatic combination can include, in weight percent of the final microbial product, no more than 50% of the at least one archaeal alpha-amylase (or variant thereof). In some embodiments, the enzymatic combination can include, in weight percent of the final microbial product, no more than 75% of the at least one archaeal alpha-amylase (or variant thereof). In some additional embodiments, the enzymatic combination can include, in weight percent of the final microbial product, no more than 95, 90, 85, 80, 75, 70, 65, 60, 55, 50, 45, 40, 35, 30, 25, 20, 15, 10, 5% or less of the at least one bacterial alpha-amylase (or variant thereof). In some embodiments, the enzymatic combination can include, in weight percent of the final microbial product, no more than 25% of the at least one bacterial alpha-amylase (or variant thereof). In some embodiments, the enzymatic combination can include, in weight percent of the final microbial product, no more than 50% of the at least one bacterial alpha-amylase (or variant thereof). In some embodiments, the enzymatic combination can include, in weight percent of the final microbial product, no more than 75% of the at least one bacterial alpha-amylase (or variant thereof). In some embodiments, the enzymatic combination can include, in weight percent of the final microbial product, between 5, 10, 15, 20, 25, 30, 35, 40, 45, 50, 55, 60, 65, 70, 75, 80, 85, 90 or 95% and 95, 90, 85, 80, 75, 70, 65, 60, 55, 50, 45, 40, 35, 30, 25, 20, 15, 10, 5% of the at least one archaeal alpha-amylase (or variant thereof). In some embodiments, the enzymatic combination can include, in weight percent of the final microbial product, between 5, 10, 15, 20, 25, 30, 35, 40, 45, 50, 55, 60, 65, 70, 75, 80, 85, 90 or 95% and 95, 90, 85, 80, 75, 70, 65, 60, 55, 50, 45, 40, 35, 30, 25, 20, 15, 10, 5% of the at least one bacterial alpha-amylase (or variant thereof). In some specific embodiments, enzymatic combination can include, in weight percent of the final microbial product, in weight percent of the final microbial product, 25% of the at least one archaeal alpha-amylase (or variant thereof) and 75% of the at least one bacterial alpha-amylase (or variant thereof). In some specific embodiments, enzymatic combination can include, in weight percent of the final microbial product, in weight percent of the final microbial product, 50% of the at least one archaeal alpha-amylase (or variant thereof) and 50% of the at least one bacterial alpha-amylase (or variant thereof). In some specific embodiments, enzymatic combination can include, in weight percent of the final microbial product, in weight percent of the final microbial product, 75% of the at least one archaeal alpha-amylase (or variant thereof) and 25% of the at least one bacterial alpha-amylase (or variant thereof).

In embodiments in which the at least one archaeal alpha-amylase (or variant thereof) is provided in an inactivated form (e.g. inactivated whole cell yeasts for example) and the at least one bacterial alpha-amylase (or variant thereof) is provided in a substantially purified form, the enzymatic combination can include, in function of the weight of dry corn, 0.001, 0.002, 0.003, 0.004, 0.005, 0.006, 0.007, 0.008, 0.009, 0.01% w/w or more of the at least one bacterial alpha-amylase and/or 0.001, 0.002, 0.003, 0.004, 0.005, 0.006, 0.007, 0.008, 0.009, 0.01, 0.011, 0.012, 0.013, 0.014, 0.015, 0.016, 0.017, 0.018, 0.019, 0.02% dcw/w or more of the at least one archaeal alpha-amylase. In embodiments in which the at least one archaeal alpha-amylase (or variant thereof) is provided in an inactivated form (e.g. inactivated whole cell yeasts for example) and the at least one bacterial alpha-amylase (or variant thereof) is provided in a substantially purified form, the enzymatic combination can include, in function of the weight of dry corn, 0.001, 0.002, 0.003, 0.004, 0.005, 0.006, 0.007, 0.008, 0.009, 0.01% w/w or more of the at least one bacterial alpha-amylase. In embodiments in which the at least one archaeal alpha-amylase (or variant thereof) is provided in an inactivated form (e.g. inactivated whole cell yeasts for example) and the at least one bacterial alpha-amylase (or variant thereof) is provided in a substantially purified form, the enzymatic combination can include, in function of the weight of dry corn, 0.001, 0.002, 0.003, 0.004, 0.005, 0.006, 0.007, 0.008, 0.009, 0.01, 0.011, 0.012, 0.013, 0.014, 0.015, 0.016, 0.017, 0.018, 0.019, 0.02% dcw/w or more of the at least one archaeal alpha-amylase.

In some embodiments, the enzymatic combination comprises a substantially purified archaeal (or variant thereof) and/or bacterial alpha-amylase (or variant thereof). In some additional embodiments, the enzymatic combination comprises the at least one archaeal alpha-amylase (or variant thereof) in a substantially purified form and a microbial product providing the at least one bacterial alpha-amylase (or variant thereof). In some additional embodiments, the enzymatic combination comprises a microbial product comprising the at least one archaeal alpha-amylase (or variant thereof) and the at least one bacterial alpha-amylase (or variant thereof) provided in a substantially purified form. In some additional embodiments, the enzymatic combination comprises the at least one archaeal alpha-amylase (or variant thereof) and the at least one bacterial alpha-amylase (or variant thereof) both in a substantially purified form.

The present disclosure also provides a kit for the liquefaction of the biomass. The kit comprising the at least one archaeal alpha-amylase as defined herein and the at least one bacterial alpha-amylase as defined herein. The at least one archaeal alpha-amylase can be provided in the same or a different containing than the at least one bacterial alpha-amylase. In an embodiment, the at least one archaeal alpha-amylase can be provided: in a substantially purified form; by the recombinant microbial host cell described herein; by the inactivated microbial product described herein; and/or by the first subpopulation of recombinant microbial host cells described herein. In another embodiment, the at least one bacterial alpha-amylase is provided: in a substantially purified form; by the recombinant microbial host cell described herein; by the inactivated microbial product described herein; and/or by the second subpopulation of recombinant microbial host cells described herein. The kit can optionally include instructions on how to obtain a hydrolyzed liquefaction medium from the biomass. The kit can optionally include additional enzymes which can be used to obtain the hydrolyzed liquefaction medium.

### Process for Making a Hydrolyzed Liquefaction Medium and Obtaining a Fermentation Product

The alpha-amylases as well as the enzyme combinations described herein can be used to break down starch and/or dextrins that may be present in the liquefaction medium into smaller molecules. The alpha-amylases as well as the enzyme combinations can be provided as an additive to a liquefaction process to provide an hydrolyzed liquefaction medium. The hydrolyzed liquefaction medium can optionally be fermented by a fermenting yeast to provide a fermentation product.

As used herein, a “liquefaction medium” or “slurry” (when the biomass comprises corn) refers to a medium which is intended to be treated in order to render more easily fermentable by the fermenting yeast. In some embodiments, the liquefaction medium is mechanically treated to reduce the size of its particles of the medium. In such embodiment, the liquefaction medium is referred to as a raw liquefaction medium. In other embodiments, the liquefaction medium is thermally and enzymatically treated to cause hydrolysis (e.g., the liquefaction) of starch it contains. In such embodiment, after the liquefaction step, an hydrolyzed liquefaction medium will be obtained.

A “fermentation medium” comprises a raw or a hydrolyzed liquefaction medium and the fermenting yeast, optionally in combination with other components. In some embodiments, the fermentation medium includes nutrients used by the fermenting yeast during the fermentation process. Components of the fermentation medium may include a carbohydrate source, a phosphorous source and a nitrogen source. The medium can optionally include micronutrients (such as vitamins and minerals), fatty acids, nitrogen, amino acids or a combination thereof. The liquefaction process of the present application comprises contacting a liquefaction medium (or a slurry) to be hydrolyzed with the alpha-amylase(s) (optionally provided in an enzyme combination), the recombinant microbial host cell, the population and/or the microbial product comprising the alpha-amylase(s) (optionally provided in an enzyme combination). Prior to liquefaction, the liquefaction medium can comprise starch (in a gelatinized or raw form). In some embodiments, the liquefaction medium is derived from a slurry such as a corn slurry (in a gelatinized or raw form). The starch present in the liquefaction medium prior to the contact with the alpha-amylase(s) (optionally provided in an enzyme combination) can be provided in a raw form (e.g., non-gelatized) or a gelatinized form. The liquefaction medium or the slurry can be provided from various biomass sources. For example, the biomass from which the mass can be obtained can include, but is not limited to, starch, sugar and lignocellulosic materials. Starch materials can include, but are not limited to, slurries such as corn, wheat, rye, barley, rice, or milo. Sugar materials can include, but are not limited to, sugar beets, artichoke tubers, sweet sorghum, molasses or cane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-protein, extensin, and pro line-rich proteins).

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; succulents, such as but not limited to, agave; and forestry wastes, such as but not limited to, recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Other lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

It will be appreciated that suitable lignocellulosic material may be any feedstock that contains soluble and/or insoluble cellulose, where the insoluble cellulose may be in a crystalline or non-crystalline form. In various embodiments, the lignocellulosic biomass comprises, for example, wood, corn, corn stover, sawdust, bark, molasses, sugarcane, leaves, agricultural and forestry residues, grasses such as switchgrass, ruminant digestion products, municipal wastes, paper mill effluent, newspaper, cardboard or combinations thereof. Paper sludge is also a viable feedstock for lactate or acetate production. Paper sludge is solid residue arising from pulping and paper-making, and is typically removed from process wastewater in a primary clarifier. The cost of disposing of wet sludge is a significant incentive to convert the material for other uses, such as conversion to ethanol. Processes provided by the present invention are widely applicable. Moreover, the fermentation products may be used to produce ethanol or higher value added chemicals, such as organic acids, aromatics, esters, acetone and polymer intermediates.

In some embodiments, in order to favor or optimize the hydrolysis of starch during the liquefaction process, it is possible to include, besides the enzymatic combination, one or more further exogenous enzyme having amylolytic activity in the liquefaction medium or slurry. However, in alternative embodiments, the presence of the enzymatic combination in the liquefaction medium or slurry reduces or alleviates the need to supplement the liquefaction medium or the slurry with another amylolytic enzymes in order to achieve the hydrolysis of the starch molecules and obtain a liquefied liquefaction medium.

The alpha-amylase(s) (optionally provided in an enzyme combination) is contacted with the liquefaction medium (which can be a slurry) under a condition to generate an hydrolyzed liquefaction medium (which can be an hydrolyzed slurry). When a combination is used, the archaeal alpha-amylase (or variant thereof) can be added to the liquefaction medium simultaneously with or sequentially to the bacterial alpha-amylase (or variant thereof). In some embodiments, the archaeal and/or bacterial alpha-amylase (or variants thereof) is/are contacted with a liquefaction medium or a slurry which has not been submitted to a heat treatment step (and in some embodiments which is not intended to be submitted to a heat treatment step). In such instances, the archaeal and/or bacterial alpha-amylase (or variants thereof) is/are contacted with an untreated liquefaction medium or an untreated slurry under a condition so as to generate the hydrolyzed liquefaction medium. In some embodiments, the archaeal and/or bacterial alpha-amylase (or variants thereof) is/are contacted with a liquefaction medium or a slurry which has not yet been submitted to a heat treatment step but is intended to be submitted to such heat treatment step. In such instances, the archaeal and/or bacterial alpha-amylase (or variants thereof) is/are contacted with an untreated liquefaction medium or untreated slurry prior to the heat treatment step. The combination of the activity of the enzyme combination and the heat treatment step can be used to generate the hydrolyzed liquefaction medium.

In other embodiments, the archaeal and/or bacterial alpha-amylase (or variants thereof) is/are contacted with a liquefaction medium or a slurry which has already been submitted to a previous heat treatment step. In such instances, the archaeal and/or bacterial alpha-amylase (or variants thereof) is/are contacted with a gelatinized liquefaction medium or gelatinized slurry as the heat treatment would have favored at least partial disruption of the starch molecules which are present in the raw liquefaction medium/raw slurry (to provide a gelatinized liquefaction medium/slurry). In some embodiments, the contact between the gelatinized liquefaction medium and the archaeal and/or bacterial alpha-amylase (or variants thereof) can occur during the heat treatment step (at least in part). In some embodiments, the archaeal and/or bacterial alpha-amylase (or variants thereof) can be added to the liquefaction medium in the liquefaction process prior to, during and/or after a heat treatment has been applied. In some embodiments, the archaeal and/or bacterial alpha-amylase (or variants thereof) is/are used to limit or prevent the recrystallization or retrogradation of the starch molecules after the heat treatment and prior to the fermentation.

It will be recognized that the amount and the number of doses of the archaeal and/or bacterial alpha-amylase (or variants thereof) to the liquefaction medium can be adjusted in function of the properties of the liquefaction medium used (amount of total solids, amount of starch, amount of gelatinized starch, presence or absence of the heat treatment step). The liquefaction process of the present disclosure can include adding one or more doses of the archaeal and/or bacterial alpha-amylase (or variants thereof) to the liquefaction medium. When a heat treatment is used during the liquefaction process, the one or more doses of the archaeal and/or bacterial alpha-amylase (or variants thereof) can be added prior to the heat treatment, during the heat treatment, after the heat treatment or any combination thereof. In a specific embodiment, the archaeal and/or bacterial alpha-amylase (or variants thereof) is/are added to the liquefaction medium prior to the heat treatment step.

As indicated herein, the liquefaction process can be performed entirely on an untreated liquefaction medium. However, in some embodiments, it may be advantageous to include a heat treatment step to the liquefaction process to liquefy, at least in part, a liquefaction medium comprising gelatinized starch molecules. The heat treatment step can improve the conversion of the starch molecules into dextrins and/or can reduce the time required to complete the liquefaction. The heat treatment step can include submitting the liquefaction medium (which may or may not include the enzyme combination) to a liquefaction temperature and for a liquefaction time period. In some embodiments, the liquefaction of starch occurs in the presence of recombinant microbial host cells and/or the microbial product described herein. In some embodiments, the liquefaction temperature is at least about 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 95° C., 100° C., 105° C. or more can be used. In some further embodiments, the liquefaction temperature is between about 60° C. to 85° C. In some further embodiments, the liquefaction temperature is between about 70° C. to 75° C. In some further embodiments, the liquefaction temperature is between about 80° C. to 85° C. When the liquefaction temperature is between about 60° C. to 85° C. it can be maintained for a liquefaction time of about 60 minutes or more.

In some additional embodiments, a jet cooker can be used to provide the heat treatment step. In such embodiments, the liquefaction temperature can be at least about 85° C., 86° C., 87° C., 88° C., 89° C., 90° C., 91° C., 92° C., 93° C., 94° C. 95° C., 96° C., 97° C., 98° C., 99° C., 100° C., 101° C. 102° C., 103° C., 104° C., 105° C., 106° C., 107° C., 108° C., 109° C., 110° C. or more. Still in such embodiment, the liquefaction temperature can be maintained for a liquefaction time of about 1 minute or more.

The liquefaction process can be conducted until a specific viscosity and/or dextrose equivalent is obtained in the liquefied liquefaction medium. As such, the process can include determining the viscosity and/or the dextrose equivalent of the liquefaction medium. This determination can be made to assess whether it may be advisable to prolong the liquefaction process, add one or more further dose of the archaeal and/or bacterial alpha-amylase of the enzyme combination and/or submit the liquefaction medium to one or more heat treatment step. This determination can also be made to determine if the hydrolyzed liquefaction medium is ready to be submitted to a subsequent fermentation.

The hydrolyzed liquefaction medium or hydrolyzed slurry that is obtained at the completion of the liquefaction process with the combinations (which can be, in some embodiments, initially conducted on a liquefaction medium comprising a corn slurry having 30-33% solids) can have a lower maximal viscosity than a corresponding hydrolyzed slurry that was obtained under similar conditions but in the absence of the bacterial alpha-amylases (e.g., in the presence of the archaeal alpha-amylases only). The hydrolyzed liquefaction medium or hydrolyzed slurry that is obtained at the completion of the liquefaction process with the combinations (which can be, in some embodiments, initially conducted on a liquefaction medium comprising a corn slurry having 30-33% solids) can have a maximal viscosity at least 5, 10, 15, 20, 30, 40, 50 or 60% lower than a corresponding hydrolyzed slurry that was obtained under similar conditions but in the absence of the bacterial alpha-amylases (e.g., in the presence of the archaeal alpha-amylases only).

The hydrolyzed liquefaction medium or hydrolyzed slurry that is obtained at the completion of the liquefaction process with the enzyme combinations (which can be, in some embodiments, initially conducted on a liquefaction medium comprising a corn slurry having 30-33% solids) can have a lower maximal viscosity than a corresponding hydrolyzed slurry that was obtained under similar conditions but in the absence of the archaeal alpha-amylases (e.g., in the presence of the bacterial alpha-amylases only). The hydrolyzed liquefaction medium or hydrolyzed slurry that is obtained at the completion of the liquefaction process with the enzyme combination (which can be, in some embodiments, initially conducted on a liquefaction medium comprising a corn slurry having 30-33% solids) can have a maximal viscosity at least 10, 20, 30, 40 or 50% lower than a corresponding hydrolyzed slurry that was obtained under similar conditions but in the absence of the archaeal alpha-amylases (e.g., in the presence of the bacterial alpha-amylases only).

The hydrolyzed liquefaction medium or hydrolyzed slurry that is obtained at the completion of the liquefaction process with the enzyme combination (which can be, in some embodiments, initially conducted on a liquefaction medium comprising a corn slurry having 30-33% solids) can have a higher dextrose equivalent than a corresponding hydrolyzed slurry that was obtained under similar conditions but in the absence of the bacterial alpha-amylases (e.g., in the presence of the archaeal alpha-amylases only). The hydrolyzed liquefaction medium or hydrolyzed slurry that is obtained at the completion of the liquefaction process with the enzyme combination (which can be, in some embodiments, initially conducted on a liquefaction medium comprising a corn slurry having 30-33% solids) can have a dextrose equivalent that is at least 1, 2, 3, 4, 5, 5, 6, 7, 8, 9, 10% or higher than a corresponding hydrolyzed slurry that was obtained under similar conditions but in the absence of the bacterial alpha-amylases (e.g., in the presence of the archaeal alpha-amylases only).

The hydrolyzed liquefaction medium or hydrolyzed slurry that is obtained at the completion of the liquefaction process with the enzyme combination (which can be, in some embodiments, initially conducted on a liquefaction medium comprising a corn slurry having 30-33% solids) can have a higher dextrose equivalent than a corresponding hydrolyzed slurry that was obtained under similar conditions but in the absence of the archaeal alpha-amylases (e.g., in the presence of the bacterial alpha-amylases only). The hydrolyzed liquefaction medium or hydrolyzed slurry that is obtained at the completion of the liquefaction process with the enzyme combination (which can be, in some embodiments, initially conducted on a liquefaction medium comprising a corn slurry having 30-33% solids) can have a dextrose equivalent that is at least 0.1, 0.2, 0.3, 0.4, 0.5, 0.6, 0.7, 0.8, 0.0, 1.0, 1.1, 1.2, 1.3, 1.4, 1.5% or higher than a corresponding hydrolyzed slurry that was obtained under similar conditions but in the absence of the archaeal alpha-amylases (e.g., in the presence of the bacterial alpha-amylases only). Once the hydrolyzed liquefaction medium has been obtained, it can be supplemented with a fermenting yeast (which can be wild-type or genetically modified) to perform a fermentation to obtain a fermentation product. The fermentation product intended to be obtained during the fermentation process can be an alcohol, such as, for example, ethanol, isopropanol, n-propanol, 1-butanol, methanol, acetone, 1,3-propanediol and/or 1,2-propanediol. In an embodiment, the liquefied liquefaction medium is used to make an alcohol (such as ethanol) as the fermentation product.

The fermentation process can be performed at temperatures of at least about 25° C., about 28° C., about 30° C., about 31° C., about 32° C., about 33° C., about 34° C., about 35° C., about 36° C., about 37° C., about 38° C., about 39° C., about 40° C., about 41° C., about 42° C., or about 50° C.

In some embodiments, the fermenting step is conducted under anaerobic conditions. As described above, yeast tends to undergo fermentation processes while under anaerobic conditions, while it tends to undergo propagation processes while under aerobic conditions. As used herein, “anaerobic conditions” means that the liquefaction medium is under an oxygen-poor environment. An oxygen-poor environment may have an oxygen concentration below that of air. For example, the concentration of oxygen may be below 21%, 20%, 15%. 10%, 9%, 8%, 7%, 6%, 5%, 4%, 3%, 2% or 1% by volume.

In embodiments in which the fermentation product is ethanol, the hydrolyzed liquefaction medium or hydrolyzed slurry that is obtained at the completion of the liquefaction process with the alpha-amylases and/or the enzyme combination (which can be, in some embodiments, initially conducted on a liquefaction medium comprising a corn slurry having 30-33% solids) can generate, during fermentation, a higher ethanol yield than a corresponding hydrolyzed slurry that was obtained under similar conditions but in the absence of the bacterial alpha-amylases (e.g., in the presence of the archaeal alpha-amylases only). The ethanol yield obtained from the fermentation of a slurry liquefied with the enzyme combination can be at least 0.1, 0.2, 0.3, 0.4, 0.5, 0.6, 0.7, 0.8, 0.9, 1.0, 1.1, 1.2, 1.3, 1.4, 1.5% or higher than the ethanol yield obtained from the fermentation of a slurry under similar conditions but in the absence of the bacterial alpha-amylases (e.g., in the presence of the archaeal alpha-amylases only).

In embodiments in which the fermentation product is ethanol, the hydrolyzed liquefaction medium or hydrolyzed slurry that is obtained at the completion of the liquefaction process with the alpha-amylases and/or the enzyme combinations (which can be, in some embodiments, initially conducted on a liquefaction medium comprising a corn slurry having 30-33% solids) can generate, during fermentation, a higher ethanol yield/fermentation solids than a corresponding hydrolyzed slurry that was obtained under similar conditions but in the absence of the bacterial alpha-amylases (e.g., in the presence of the archaeal alpha-amylases only).

The ethanol yield/fermentation solids obtained from the fermentation of a slurry liquefied with the enzyme combination can be at least 0.1, 0.2, 0.3, 0.4, 0.5, 0.6, 0.7, 0.8, 0.9, 1.0, 1.1, 1.2, 1.3, 1.4, 1.5% or higher than the ethanol yield/fermentation solids obtained from the fermentation of a slurry under similar conditions but in the absence of the bacterial alpha-amylases (e.g., in the presence of the archaeal alpha-amylases only).

In embodiments in which the fermentation product is ethanol, the hydrolyzed liquefaction medium or hydrolyzed slurry that is obtained at the completion of the liquefaction process with the alpha-amylases and/or the enzyme combinations (which can be, in some embodiments. initially conducted on a liquefaction medium comprising a corn slurry having 30-33% solids) can generate, during fermentation, a higher ethanol yield than a corresponding hydrolyzed slurry that was obtained under similar conditions but in the absence of the archaeal alpha-amylases (e.g., in the presence of the bacterial alpha-amylases only). The ethanol yield obtained from the fermentation of a slurry liquefied with the enzyme combination can be at least 0.1, 0.2, 0.3, 0.4, 0.5, 0.6, 0.7, 0.8, 0.9, 1.0, 1.1, 1.2, 1.3, 1.4, 1.5% or higher than the ethanol yield obtained from the fermentation of a slurry under similar conditions but in the absence of the archaeal alpha-amylases (e.g., in the presence of the bacterial alpha-amylases only).

In embodiments in which the fermentation product is ethanol, the hydrolyzed liquefaction medium or hydrolyzed slurry that is obtained at the completion of the liquefaction process with the alpha-amylases and/or the enzyme combinations (which can be, in some embodiments, initially conducted on a liquefaction medium comprising a corn slurry having 30-33% solids) can generate, during fermentation, a higher ethanol yield/fermentation solids than a corresponding hydrolyzed slurry that was obtained under similar conditions but in the absence of the archaeal alpha-amylases (e.g., in the presence of the bacterial alpha-amylases only).

The ethanol yield/fermentation solids obtained from the fermentation of a slurry liquefied with the enzyme combination can be at least 0.1, 0.2, 0.3, 0.4, 0.5, 0.6, 0.7, 0.8, 0.9, 1.0, 1.1, 1.2, 1.3, 1.4, 1.5% or higher than the ethanol yield/fermentation solids obtained from the fermentation of a slurry under similar conditions but in the absence of the archaeal alpha-amylases (e.g., in the presence of the bacterial alpha-amylases only).

The hydrolyzed liquefaction medium or hydrolyzed slurry that is obtained at the completion of the liquefaction process with the alpha-amylases and/or the enzyme combinations (which can be, in some embodiments, initially conducted on a liquefaction medium comprising a corn slurry having 30-33% solids) can have a higher dextrose equivalent than a corresponding hydrolyzed slurry that was obtained under similar conditions but in the absence of the archaeal alpha-amylases (e.g., in the presence of the bacterial alpha-amylases only). The hydrolyzed liquefaction medium or hydrolyzed slurry that is obtained at the completion of the liquefaction process with the enzyme combination (which can be, in some embodiments, initially conducted on a liquefaction medium comprising a corn slurry having 30-33% solids) can have a dextrose equivalent that is at least 0.1, 0.2, 0.3, 0.4, 0.5, 0.6, 0.7, 0.8, 0.0, 1.0, 1.1, 1.2, 1.3, 1.4, 1.5% higher than a corresponding hydrolyzed slurry that was obtained under similar conditions but in the absence of the archaeal alpha-amylases (e.g., in the presence of the bacterial alpha-amylases only).

The fermenting yeast can be, for example, from the genus Saccharomyces, Kluyveromyces, Arxula, Debaryomyces, Candida, Pichia, Komagataella, Phaffia, Schizosaccharomyces, Hansenula, Kloeckera, Schwanniomyces, Torula or Yarrowia. Suitable yeast species can include, for example, S. cerevisiae, S. bulderi, S. barnetti, S. exiguus, S. uvarum, S. diastaticus, S. boulardii, C. utilis, K. lactis, K. marxianus or K. fragilis. In some embodiments, the fermenting yeast is selected from the group consisting of Saccharomyces cerevisiae, Schizzosaccharomyces pombe, Candida albicans, Komagataella phaffii, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In some further embodiments, the fermenting yeast is of Saccharomyces cerevisiae, Schizzosaccharomyces pombe, Candida albicans, Komagataella phaffii, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe or Schwanniomyces occidentalis. In one particular embodiment, the fermenting yeast is Saccharomyces cerevisiae. In some embodiments, the fermenting yeast can be an oleaginous yeast cell. For example, the oleaginous yeast cell can be from the genus Blakeslea, Candida, Cryptococcus,

Cunninghamella, Lipomyces, Mortierella, Mucor, Phycomyces, Pythium, Rhodosporidum, Rhodotorula, Trichosporon or Yarrowia. In some alternative embodiment, the fermenting yeast can be an oleaginous microalgae host cell (e.g., for example, from the genus Thraustochytrium or Schizochytrium). In an embodiment, the fermenting yeast is from the genus Saccharomyces and, in some embodiments, from the species Saccharomyces cerevisiae.

In some embodiments, the fermenting yeast comprises a genetic modification (e.g., a heterologous nucleic acid molecule) for reducing the production of one or more native enzymes that function to produce glycerol or regulate glycerol synthesis, for allowing the production of a polypeptide having a lytic enzyme (including but not limited to a glucoamylase) and/or for reducing the production of one or more native enzymes that function to catabolize formate.

As used in the context of the present disclosure, the expression “reducing the production of one or more native enzymes that function to produce glycerol or regulate glycerol synthesis” refers to a genetic modification which limits or impedes the expression of genes associated with one or more native polypeptides (in some embodiments enzymes) that function to produce glycerol or regulate glycerol synthesis, when compared to a corresponding host strain which does not bear the genetic modification. In some instances, the genetic modification reduces but still allows the production of one or more native polypeptides that function to produce glycerol or regulate glycerol synthesis. In other instances, the genetic modification inhibits the production of one or more native enzymes that function to produce glycerol or regulate glycerol synthesis. In some embodiments, the fermenting yeast bears a plurality of second genetic modifications, wherein at least one reduces the production of one or more native polypeptides and at least another inhibits the production of one or more native polypeptides.

As used in the context of the present disclosure, the expression “native polypeptides that function to produce glycerol or regulate glycerol synthesis” refers to polypeptides which are endogenously found in the fermenting yeast. Native enzymes that function to produce glycerol include, but are not limited to, the GPD1 and the GPD2 polypeptide (also referred to as GPD1 and GPD2 respectively). Native enzymes that function to regulate glycerol synthesis include, but are not limited to, the FPS1 polypeptide. In an embodiment, the fermenting yeast bears a genetic modification in at least one of the gpd1 gene (encoding the GPD1 polypeptide), the gpd2 gene (encoding the GPD2 polypeptide), the fps1 gene (encoding the FPS1 polypeptide) or orthologs thereof. In another embodiment, the fermenting yeast cell bears a genetic modification in at least two of the gpd1 gene (encoding the GPD1 polypeptide), the gpd2 gene (encoding the GPD2 polypeptide), the fps1 gene (encoding the FPS1 polypeptide) or orthologs thereof. In still another embodiment, the fermenting yeast bears a genetic modification in each of the gpd1 gene (encoding the GPD1 polypeptide), the gpd2 gene (encoding the GPD2 polypeptide) and the fps1 gene (encoding the FPS1 polypeptide) or orthologs thereof.

Examples of fermenting yeasts bearing such genetic modification(s) leading to the reduction in the production of one or more native enzymes that function to produce glycerol or regulate glycerol synthesis are described in U.S. Pat. No. 11,034,967 (herein incorporated in its entirety). Preferably, the fermenting yeast cell has a genetic modification (such as a genetic deletion or insertion) only in one enzyme that functions to produce glycerol, in the gpd2 gene, which would cause the host cell to have a knocked-out gpd2 gene. In some embodiments, the fermenting yeast cell can have a genetic modification in the gpd1 gene, the gpd2 gene and the fps1 gene resulting is a fermenting yeast being knock-out for the gpd1 gene, the gpd2 gene and the fps1 gene.

Polypeptides facilitating glycerol transport include but are not limited to polypeptides having glycerol proton symporter activity. An embodiment of a polypeptide having glycerol proton symporter activity is stl1, a polypeptide encoded by a stl1 gene ortholog and/or a polypeptide encoded by a stl1 gene paralog. stl1 can be natively expressed in yeasts and fungi. stl1 genes encoding the stl1 polypeptide include, but are not limited to, Saccharomyces cerevisiae Gene ID: 852149, Candida albicans, Kluyveromyces lactis Gene ID: 2896463, Ashbya gossypii Gene ID: 4620396, Eremothecium sinecaudum Gene ID: 28724161, Torulaspora delbrueckii Gene ID: 11505245, Lachancea thermotolerans Gene ID: 8290820, Phialophora attae Gene ID: 28742143, Penicillium digitatum Gene ID: 26229435, Aspergillus oryzae Gene ID: 5997623, Aspergillus fumigatus Gene ID: 3504696, Talaromyces atroroseus Gene ID: 31007540, Rasamsonia emersonii Gene ID: 25315795, Aspergillus flavus Gene ID: 7910112, Aspergillus terreus Gene ID: 4322759, Penicillium chrysogenum Gene ID: 8310605, Alternaria alternata Gene ID: 29120952. Paraphaeosphaeria sporulosa Gene ID: 28767590, Pyrenophora tritici-repentis Gene ID: 6350281, Metarhizium robertsii Gene ID: 19259252, Isaria fumosorosea Gene ID: 30023973. Cordyceps militaris Gene ID: 18171218, Pochonia chlamydosporia Gene ID: 28856912, Metarhizium majus Gene ID: 26274087, Neofusicoccum parvum Gene ID: 19029314, Diplodia corticola Gene ID: 31017281, Verticillium dahliae Gene ID: 20711921, Colletotrichum gloeosporioides Gene ID: 18740172, Verticillium albo-atrum Gene ID: 9537052. Paracoccidioides lutzii Gene ID: 9094964, Trichophyton rubrum Gene ID: 10373998, Nannizzia gypsea Gene ID: 10032882, Trichophyton verrucosum Gene ID: 9577427, Arthroderma benhamiae Gene ID: 9523991, Magnaporthe oryzae Gene ID: 2678012, Gaeumannomyces graminis var. tritici Gene ID: 20349750, Togninia minima Gene ID: 19329524, Eutypa lata Gene ID: 19232829, Scedosporium apiospermum Gene ID: 27721841, Aureobasidium namibiae Gene ID: 25414329, Sphaerulina musiva Gene ID: 27905328 as well as Pachysolen tannophilus GenBank Accession Numbers JQ481633 and JQ481634, Saccharomyces paradoxus stl1 and Pichia sorbitophilia. In an embodiment, the stl1 polypeptide is encoded by Saccharomyces cerevisiae Gene ID: 852149. In a specific embodiment, the stl1 polypeptide is derived from Saccharomyces sp. and in further embodiments from Saccharomyces cerevisiae. In some specific embodiments, the heterologous nucleic acid molecule encoding the stl1 polypeptide, its variants or its fragments is knocked-in at the native position at which the gene of the native stl1 polypeptide is located.

As used in the context of the present disclosure, the expression “native polypeptides that function to catabolize formate” refers to polypeptides which are endogenously found in the fermenting yeast cell. Native enzymes that function to catabolize formate include, but are not limited to, the FDH1 and the FDH2 polypeptides (also referred to as FDH1 and FDH2 respectively). In an embodiment, the fermenting yeast cell bears a genetic modification in at least one of the fdh1 gene (encoding the FDH1 polypeptide), the fdh2 gene (encoding the FDH2 polypeptide) or orthologs thereof. In another embodiment, the fermenting yeast cell bears genetic modifications in both the fdh1 gene (encoding the FDH1 polypeptide) and the fdh2 gene (encoding the FDH2 polypeptide) or orthologs thereof. Examples of fermenting yeast cells bearing such genetic modification(s) leading to the reduction in the production of one or more native enzymes that function to catabolize formate are described in U.S. Pat. No. 8,956,851 (herein incorporated in its entirety). Preferably, the fermenting yeast cell has genetic modifications (such as a genetic deletion or insertion) in the fdh1 gene and in the fdh2 gene which would cause the host cell to have knocked-out fdh1 and fdh2 genes.

In an embodiment, the fermenting yeast host cell includes a genetic modification does achieve higher pyruvate formate lyase activity in the fermenting yeast. This increase in pyruvate formate lyase activity is relative to a corresponding native yeast host cell which does not include the first genetic modification. As used in the context of the present disclosure, the term “pyruvate formate lyase” or “PFL” refers to an enzyme (EC 2.3.1.54) also known as formate C-acetyltransferase, pyruvate formate-lyase, pyruvic formate-lyase and formate acetyltransferase. Pyruvate formate lyases are capable of catalyzing the conversion of coenzyme A (CoA) and pyruvate into acetyl-CoA and formate. In some embodiments, the pyruvate formate lyase activity may be increased by expressing a heterologous pyruvate formate lyase activitating enzyme and/or a pyruvate formate lyase enzymate (such as, for example PFLA and/or PFLB).

In the context of the present disclosure, the genetic modification can include the introduction of a heterologous nucleic acid molecule encoding a pyruvate formate lyase activating enzyme and/or a puryvate formate lyase enzyme, such as PFLA. Embodiments of the pyruvate formate lyase activating enzyme and of PFLA can be derived, without limitation, from the following (the number in brackets correspond to the Gene ID number): Escherichia coli (MG1655945517). Shewanella oneidensis (1706020), Bifidobacterium longum (1022452), Mycobacterium bovis (32287203), Haemophilus parasuis (7277998), Mannheimia haemolytica (15341817), Vibrio vulnificus (33955434), Cronobacter sakazakii (29456271), Vibrio alginolyticus (31649536). Pasteurella multocida (29388611), Aggregatibacter actinomycetemcomitans (31673701), Actinobacillus suis (34291363), Finegoldia magna (34165045), Zymomonas mobilis subsp. mobilis (3073423), Vibrio tubiashii (23444968), Gallibacterium anatis (10563639), Actinobacillus pleuropneumoniae serovar (4849949), Ruminiclostridium thermocellum (35805539), Cylindrospermopsis raciborskii (34474378), Lactococcus garvieae (34204939), Bacillus cytotoxicus (33895780), Providencia stuartii (31518098), Pantoea ananatis (31510290), Teredinibacter turnerae (29648846), Morganella morganii subsp. morganii (14670737), Vibrio anguillarum (77510775106), Dickeya dadantii (39379733484), Xenorhabdus bovienii (8830449), Edwardsiella ictaluri (7959196), Proteus mirabilis (6801040), Rahnella aquatilis (34350771), Bacillus pseudomycoides (34214771), Vibrio alginolyticus (29867350), Vibrio nigripulchritudo (29462895), Vibrio orientalis (25689084), Kosakonia sacchari (23844195), Serratia marcescens subsp. marcescens (23387394), Shewanella baltica (11772864), Vibrio vulnificus (2625152), Streptomyces acidiscabies (33082227), Streptomyces davaonensis (31227069), Streptomyces scabiei (24308152), Volvox carteri f. nagariensis (9616877), Vibrio breoganii (35839746), Vibrio mediterranei (34766273), Fibrobacter succinogenes subsp. succinogenes (34755395), Enterococcus gilvus (34360882), Akkermansia muciniphila (34173806), Enterobacter hormaechei subsp. Steigerwaltii (34153767), Dickeya zeae (33924935), Enterobacter sp. (32442159), Serratia odorifera (31794665), Vibrio crassostreae (31641425), Selenomonas ruminantium subsp. lactilytica (31522409), Fusobacterium necrophorum subsp. funduliforme (31520833), Bacteroides uniformis (31507008), Haemophilus somnus (233631487328), Rodentibacter pneumotropicus (31211548), Pectobacterium carotovorum subsp. carotovorum (29706463), Eikenella corrodens (29689753), Bacillus thuringiensis (29685036), Streptomyces rimosus subsp. Rimosus (29531909), Vibrio fluvialis (29387180), Klebsiella oxytoca (29377541). Parageobacillus thermoglucosidans (29237437), Aeromonas veronii (28678409), Clostridium innocuum (26150741), Neisseria mucosa (25047077), Citrobacter freundii (23337507), Clostridium bolteae (23114831), Vibrio tasmaniensis (7160642), Aeromonas salmonicida subsp. salmonicida (4995006), Escherichia coli O157: H7 str. Sakai (917728), Escherichia coli O83: H1 str. (12877392), Yersinia pestis (11742220), Clostridioides difficile (4915332), Vibrio fischeri (3278678), Vibrio parahaemolyticus (1188496), Vibrio coralliilyticus (29561946), Kosakonia cowanii (35808238), Yersinia ruckeri (29469535), Gardnerella vaginalis (99041930), Listeria fleischmannii subsp. Coloradonensis (34329629), Photobacterium kishitanii (31588205), Aggregatibacter actinomycetemcomitans (29932581), Bacteroides caccae (36116123), Vibrio toranzoniae (34373279), Providencia alcalifaciens (34346411). Edwardsiella anguillarum (33937991), Lonsdalea quercina subsp. Quercina (33074607). Pantoea septica (32455521), Butyrivibrio proteoclasticus (31781353), Photorhabdus temperata subsp. Thracensis (29598129), Dickeya solani (23246485), Aeromonas hydrophila subsp. hydrophila (4489195), Vibrio cholerae O1 biovar El Tor str. (2613623), Serratia rubidaea (32372861), Vibrio bivalvicida (32079218), Serratia liquefaciens (29904481), Gilliamella apicola (29851437), Pluralibacter gergoviae (29488654), Escherichia coli 0104: H4 (13701423), Enterobacter aerogenes (10793245), Escherichia coli (7152373), Vibrio campbellii (5555486), Shigella dysenteriae (3795967), Bacillus thuringiensis serovar konkukian (2854507), Salmonella enterica subsp. enterica serovar Typhimurium (1252488), Bacillus anthracis (1087733), Shigella flexneri (1023839), Streptomyces griseoruber (32320335), Ruminococcus gnavus (35895414), Aeromonas fluvialis (35843699), Streptomyces ossamyceticus (35815915), Xenorhabdus doucetiae (34866557), Lactococcus piscium (34864314), Bacillus glycinifermentans (34773640), Photobacterium damselae subsp. Damselae 34509297, Streptomyces venezuelae 34035779, Shewanella algae (34011413). Neisseria sicca (33952518), Chania multitudinisentens (32575347), Kitasatospora purpeofusca (32375714), Serratia fonticola (32345867), Aeromonas enteropelogenes (32325051), Micromonospora aurantiaca (32162988), Moritella viscosa (31933483), Yersinia aldovae (31912331), Leclercia adecarboxylata (31868528), Salinivibrio costicola subsp. costicola (31850688), Aggregatibacter aphrophilus (31611082), Photobacterium leiognathi (31590325), Streptomyces canus (31293262), Pantoea dispersa (29923491), Pantoea rwandensis (29806428), Paenibacillus borealis (29548601), Aliivibrio wodanis (28541257), Streptomyces virginiae (23221817), Escherichia coli (7158493), Mycobacterium tuberculosis (887973), Streptococcus mutans (1028925), Streptococcus cristatus (29901602). Enterococcus hirae (13176624), Bacillus licheniformis (3031413), Chromobacterium violaceum (24949178), Parabacteroides distasonis (5308542), Bacteroides vulgatus (5303840), Faecalibacterium prausnitzii (34753201), Melissococcus plutonius (34410474). Streptococcus gallolyticus subsp. gallolyticus (34397064), Enterococcus malodoratus (34355146), Bacteroides oleiciplenus (32503668), Listeria monocytogenes (985766), Enterococcus faecalis (1200510), Campylobacter jejuni subsp. jejuni (905864). Lactobacillus plantarum (1063963), Yersinia enterocolitica subsp. enterocolitica (4713333), Streptococcus equinus (33961143), Macrococcus canis (35294771), Streptococcus sanguinis (4807186), Lactobacillus salivarius (3978441), Lactococcus lactis subsp. lactis (1115478), Enterococcus faecium (12999835), Clostridium botulinum A (5184387), Clostridium acetobutylicum (1117164), Bacillus thuringiensis serovar konkukian (2857050), Cryobacterium flavum (35899117), Enterovibrio norvegicus (35871749), Bacillus acidiceler (34874556), Prevotella intermedia (34516987), Pseudobutyrivibrio ruminis (34419801), Pseudovibrio ascidiaceicola (34149433), Corynebacterium coyleae (34026109), Lactobacillus curvatus (33994172). Cellulosimicrobium cellulans (33980622), Lactobacillus agilis (33975995), Lactobacillus sakei (33973512), Staphylococcus simulans (32051953), Obesumbacterium proteus (29501324), Salmonella enterica subsp. enterica serovar Typhi (1247402), Streptococcus agalactiae (1014207), Streptococcus agalactiae (1013114), Legionella pneumophila subsp. pneumophila str. Philadelphia (119832735), Pyrococcus furiosus (1468475), Mannheimia haemolytica (15340992), Thalassiosira pseudonana (7444511), Thalassiosira pseudonana (7444510). Streptococcus thermophilus (31940129), Sulfolobus solfataricus (1454925), Streptococcus iniae (35765828), Streptococcus iniae (35764800), Bifidobacterium thermophilum (31839084). Bifidobacterium animalis subsp. lactis (29695452), Streptobacillus moniliformis (29673299), Thermogladius calderae (13013001), Streptococcus oralis subsp. tigurinus (31538096), Lactobacillus ruminis (29802671), Streptococcus parauberis (29752557), Bacteroides ovatus (29454036), Streptococcus gordonii str. Challis substr. CH1 (25052319), Clostridium botulinum B str. Eklund 17B (19963260), Thermococcus litoralis (16548368), Archaeoglobus sulfaticallidus (15392443), Ferroglobus placidus (8778929), Archaeoglobus profundus (8739370), Listeria seeligeri serovar 1/2b (32488230), Bacillus thuringiensis (31632063). Rhodobacter capsulatus (31491679), Clostridium botulinum (29749009), Clostridium perfringens (29571530), Lactococcus garvieae (12478921), Proteus mirabilis (6799920), Lactobacillus animalis (32012274), Vibrio alginolyticus (29869205), Bacteroides thetaiotaomicron (31617701), Bacteroides thetaiotaomicron (31617140), Bacteroides cellulosilyticus (29608790), Bacteroides ovatus (29453452), Bacillus mycoides (29402181), Chlamydomonas reinhardtii (5726206), Fusobacterium periodonticum (35833538). Selenomonas flueggei (32477557), Selenomonas noxia (32475880), Anaerococcus hydrogenalis (32462628), Centipeda periodontii (32173931), Centipeda periodontii (32173899), Streptococcus thermophilus (31938326), Enterococcus durans (31916360). Fusobacterium nucleatum (31730399), Anaerostipes hadrus (31625694), Anaerostipes hadrus (31623667), Enterococcus haemoperoxidus (29838940), Gardnerella vaginalis (29692621), Streptococcus salivarius (29397526), Klebsiella oxytoca (29379245), Bifidobacterium breve (29241363), Actinomyces odontolyticus (25045153), Haemophilus ducreyi (24944624), Archaeoglobus fulgidus (24793671), Streptococcus uberis (24161511). Fusobacterium nucleatum subsp. animalis (23369066), Corynebacterium accolens (23249616), Archaeoglobus veneficus (10394332), Prevotella melaninogenica (9497682), Aeromonas salmonicida subsp. salmonicida (4997325), Pyrobaculum islandicum (4616932), Thermofilum pendens (4600420), Bifidobacterium adolescentis (4556560), Listeria monocytogenes (986485), Bifidobacterium thermophilum (35776852), Methanothermobacter sp. CaT2 (24854111), Streptococcus pyogenes (901706), Exiguobacterium sibiricum (31768748), Clostridioides difficile (4916015), Clostridioides difficile (4913022), Vibrio parahaemolyticus (1192264), Yersinia enterocolitica subsp. enterocolitica (4712948), Enterococcus cecorum Bifidobacterium (29475065), pseudolongum (34879480), Methanothermus fervidus (9962832), Methanothermus fervidus (9962056), Corynebacterium simulans (29536891), Thermoproteus uzoniensis (10359872), Vulcanisaeta distributa (9752274), Streptococcus mitis (8799048), Ferroglobus placidus (8778420), Streptococcus suis (8153745), Clostridium novyi (4541619), Streptococcus mutans (1029528), Thermosynechococcus elongatus (1010568), Chlorobium tepidum (1007539), Fusobacterium nucleatum subsp. nucleatum (993139), Streptococcus pneumoniae (933787), Clostridium baratii (31579258), Enterococcus mundtii (31547246). Prevotella ruminicola (31500814). Aeromonas hydrophila subsp. hydrophila (4490168), Aeromonas hydrophila subsp. hydrophila (4487541), Clostridium acetobutylicum (1117604), Chromobacterium subtsugae (31604683), Gilliamella apicola (29849369), Klebsiella pneumoniae subsp. pneumoniae (11846825), Enterobacter cloacae subsp. cloacae (9125235), Escherichia coli (7150298), Salmonella enterica subsp. enterica serovar Typhimurium (1252363), Salmonella enterica subsp. enterica serovar Typhi (1247322), Bacillus cereus (1202845), Bacteroides thetaiotaomicron (1074343), Bacteroides thetaiotaomicron (1071815), Bacillus coagulans (29814250), Bacteroides cellulosilyticus (29610027), Bacillus anthracis (2850719), Monoraphidium neglectum (25735215), Monoraphidium neglectum (25727595), Alloscardovia omnicolens (35868062), Actinomyces neuii subsp. neuii (35867196), Acetoanaerobium sticklandii (35557713), Exiguobacterium undae (32084128), Paenibacillus pabuli (32034589), Paenibacillus etheri (32019864), Actinomyces oris (31655321), Vibrio alginolyticus (31651465), Brochothrix thermosphacta (29820407), Lactobacillus sakei subsp. sakei (29638315), Anoxybacillus gonensis (29574914), variants thereof as well as fragments thereof. In an embodiment, the PFLA protein is derived from the genus Bifidobacterium and in some embodiments from the species Bifidobacterium adolescentis. In an embodiment, the heterologous nucleic acid molecule encoding the PFLA protein is present in at least one, two, three, four, five or more copies in the fermenting yeast. In still another embodiment, the heterologous nucleic acid molecule encoding the PFLA protein is present in no more than five, four, three, two or one copy/ies in the fermenting yeast.

In the context of the present disclosure, the fermenting yeast host cell has a genetic modification encoding a formate acetyltransferase enzyme and/or a puryvate formate lyase enzyme, such as PFLB. Embodiments of PFLB can be derived, without limitation, from the following (the number in brackets correspond to the Gene ID number): Escherichia coli (945514), Shewanella oneidensis (1170601), Actinobacillus suis (34292499), Finegoldia magna (34165044), Streptococcus cristatus (29901775), Enterococcus hirae (13176625), Bacillus (3031414), Providencia alcalifaciens (34345353), Lactococcus garvieae (34203444). Butyrivibrio proteoclasticus (31781354), Teredinibacter turnerae (29651613), Chromobacterium violaceum (24945652), Vibrio campbellii (5554880), Vibrio campbellii (5554796), Rahnella aquatilis HX2 (34351700), Serratia rubidaea (32375076), Kosakonia sacchari SP1 (23845740), Shewanella baltica (11772863), Streptomyces acidiscabies (33082309), Streptomyces davaonensis (31227068), Parabacteroides distasonis (5308541), Bacteroides vulgatus (5303841), Fibrobacter succinogenes subsp. succinogenes (34755392), Photobacterium damselae subsp. Damselae (34512678), Enterococcus gilvus (34361749), Enterococcus gilvus (34360863), Enterococcus malodoratus (34355213), Enterococcus malodoratus (34354022), Akkermansia muciniphila (34174913), Lactobacillus curvatus (33995135), Dickeya zeae (33924934), Bacteroides oleiciplenus (32502326). Micromonospora aurantiaca (32162989), Selenomonas ruminantium subsp. lactilytica (31522408), Fusobacterium necrophorum subsp. funduliforme (31520832), Bacteroides uniformis (31507007), Streptomyces rimosus subsp. Rimosus (29531908), Clostridium innocuum (26150740), Haemophilus] ducreyi (24944556), Clostridium bolteae (23114829), Vibrio tasmaniensis (7160644), Aeromonas salmonicida subsp. salmonicida (4997718). Listeria monocytogenes (986171), Enterococcus faecalis (1200511), Lactobacillus plantarum (1064019), Vibrio fischeri (3278780), Lactobacillus sakei (33973511), Gardnerella vaginalis (9904192), Vibrio vulnificus (33954428), Vibrio toranzoniae (34373229), Anaerostipes hadrus (34240161), Edwardsiella anguillarum (33940299), Edwardsiella anguillarum (33937990), Lonsdalea quercina subsp. Quercina (33074710), Enterococcus faecium (12999834), Aeromonas hydrophila subsp. hydrophila (4489100), Clostridium acetobutylicum (1117163), Escherichia coli (7151395). Shigella dysenteriae (3795966), Bacillus thuringiensis serovar konkukian (2856201), Salmonella enterica subsp. enterica serovar Typhimurium (1252491). Shigella flexneri (1023824), Streptomyces griseoruber (32320336), Cryobacterium flavum (35898977), Ruminococcus gnavus (35895748), Bacillus acidiceler (34874555), Lactococcus piscium (34864362), Vibrio mediterranei (34766270), Faecalibacterium prausnitzii (34753200), Prevotella intermedia (34516966), Photobacterium damselae subsp. Damselae (34509286), Pseudobutyrivibrio ruminis (34419894), Melissococcus plutonius (34408953). Streptococcus gallolyticus subsp. gallolyticus (34398704), Enterobacter hormaechei subsp. Steigerwaltii (34155981), Enterobacter hormaechei subsp. Steigerwaltii (34152298), Streptomyces venezuelae (34036549), Shewanella algae (34009243), Lactobacillus agilis (33976013), Streptococcus equinus (33961013), Neisseria sicca (33952517), Kitasatospora purpeofusca (32375782), Paenibacillus borealis (29549449), Vibrio fluvialis (29387150), Aliivibrio wodanis (28542465), Aliivibrio wodanis (28541256), Escherichia coli (7157421). Salmonella enterica subsp. enterica serovar Typhi (1247405), Yersinia pestis (1174224), Yersinia enterocolitica subsp. enterocolitica (4713334), Streptococcus suis (8155093). Escherichia coli (947854), Escherichia coli (946315), Escherichia coli (945513), Escherichia coli (948904), Escherichia coli (917731), Yersinia enterocolitica subsp. enterocolitica (4714349), variants thereof as well as fragments thereof. In an embodiment, the PFLB protein is derived from the genus Bifidobacterium and in some embodiments from the specifies Bifidobacterium adolescentis. In an embodiment, the heterologous nucleic acid molecule encoding the PFLB protein is present in at least one, two, three, four, five or more copies in the fermenting yeast. In still another embodiment, the heterologous nucleic acid molecule encoding the PFLB protein is present in no more than five, four, three, two or one copy/ies in the fermenting yeast.

In some embodiments, the fermenting yeast host cell comprises a first genetic modification for expressing a PFLA protein, a PFLB protein or a combination. In a specific embodiment, the fermenting yeast host cell comprises a first genetic modification for expressing a PFLA protein and a PFLB protein which can, in some embodiments, be provided on distinct heterologous nucleic acid molecules. As indicated below, the fermenting yeast host cell can also include additional genetic modifications to provide or increase its ability to transform acetyl-CoA into an alcohol such as ethanol.

Alternatively or in combination, the fermenting yeast host cell can bear one or more genetic modification for utilizing acetyl-CoA for example, by providing or increasing acetaldehyde and/or alcohol dehydrogenase activity. Acetyl-coA can be converted to an alcohol such as ethanol using first an acetaldehyde dehydrogenase and then an alcohol dehydrogenase.

Alcohol dehydrogenases are classified in EC number 1.1.1.1 and catalyze the conversion of a primary or a secondary alcohol with NAD (+) in an aldehyde or a ketone with NADH. In some specific embodiments, the one or more first genetic modifications comprise a genetic modification for overexpressing a native polypeptide having alcohol dehydrogenase activity and/or expressing a heterologous polypeptide having alcohol dehydrogenase activity. The recombinant yeast cell of the present disclosure can include a genetic modification for overexpressing a native polypeptide having alcohol dehydrogenase activity. The recombinant yeast cell of the present disclosure can include a genetic modification for expressing a heterologous polypeptide having alcohol dehydrogenase activity. In some embodiments, recombinant yeast cell comprising the one or more first genetic modification for increasing alcohol dehydrogenase activity can be further modified to reduce the expression and/or inactivate at least one copy of a native gene encoding a polypeptide having alcohol dehydrogenase activity. Heterologous alcohol dehydrogenases includes, but are not limited to the adhA polypeptide, a polypeptide encoded by an adha gene ortholog or gene paralog, the adhB polypeptide or a polypeptide encoded by an adhb gene ortholog or gene paralog. In an embodiment, the polypeptide having alcohol dehydrogenase activity is derived from a Zymomonas genus and, in specific embodiments, from Zymomonas mobilis. In another embodiment, heterologous alcohol dehydrogenases includes, but are not limited to the ADH polypeptide and a polypeptide encoded by an adh gene ortholog or gene paralog. In an embodiment, the polypeptide having alcohol dehydrogenase activity is derived from a Sporotrichum genus and, in specific embodiments, from Sporotrichum pulverulentum.

In some embodiments, the one or more first genetic modifications include a genetic modification capable of causing or which causes a modulation (and is some embodiments a decrease) in aldehyde dehydrogenase (NADP(+)) activity. Aldehyde dehydrogenase (NADP(+)) are classified in EC number 1.2.1.4 and catalyze the conversion of an aldehyde with NADP(+) in carboxylate with NADPH. In some specific embodiments, the one or more first genetic modifications comprise a genetic modification for reducing the expression and/or inactivating at least one copy of a native gene encoding a polypeptide having aldehyde dehydrogenase (NADP(+)) activity. In some specific embodiments, the one or more first genetic modifications comprise a genetic modification for reducing the expression and/or inactivating at least one copy of a native gene encoding an ald6 polypeptide.

In some embodiments, the one or more first genetic modifications include a genetic modification capable of causing or which causes a modulation (and is some embodiments an increase) in acetaldehyde (acetylating) dehydrogenase activity. Acetaldehyde dehydrogenases (acetylating) are classified in EC number 1.2.1.10 and catalyze the conversion of an acetaldehyde, CoA and NAD (+) in acetyl-CoA and NADH. In some specific embodiments, the one or more first genetic modifications comprise a genetic modification for overexpressing a native polypeptide having acetaldehyde dehydrogenase (acetylating) activity and/or expressing a heterologous polypeptide having acetaldehyde dehydrogenase (acetylating) activity. The recombinant yeast cell of the present disclosure can include a genetic modification for overexpressing a native polypeptide having acetaldehyde dehydrogenase (acetylating) activity. The recombinant yeast cell of the present disclosure can include a genetic modification for expressing a heterologous polypeptide having acetaldehyde dehydrogenase (acetylating).

For example, the genetic modification can comprise introducing a heterologous nucleic acid molecule encoding an acetaldehyde dehydrogenase. In another example, the genetic modification can comprise introducing a heterologous nucleic acid molecule encoding an alcohol dehydrogenase. In still another example, the genetic modification can comprise introducing at least two heterologous nucleic acid molecules, a first one encoding a heterologous acetaldehyde dehydrogenase and a second one encoding a heterologous alcohol dehydrogenase. In another embodiment, the genetic modification comprises introducing a heterologous nucleic acid d encoding a heterologous bifunctional acetylaldehyde/alcohol dehydrogenases (AADH) such as those described in U.S. Pat. No. 8,956,851 and U.S. patent application No. 20160194669 (both incorporated herein in their entirety). Heterologous AADHs of the present disclosure include, but are not limited to, the ADHE polypeptides or a polypeptide encoded by an adhe gene ortholog.

The fermenting yeast host cell can be further genetically modified to allow for the production of additional heterologous polypeptides. In an embodiment, the fermenting yeast cell can be used for the production of an enzyme, and especially an enzyme involved in the cleavage or hydrolysis of its substrate (e.g., a lytic enzyme and, in some embodiments, a saccharolytic enzyme). In still another embodiment, the enzyme can be a glycoside hydrolase. In the context of the present disclosure, the term “glycoside hydrolase” refers to an enzyme involved in carbohydrate digestion, metabolism and/or hydrolysis, including amylases (other than those described above), cellulases, hemicellulases (including, but not limited to xylanases, xylosidases, arabinofuranosidases, galactosidases and/or endoglucanase), cellulolytic and amylolytic accessory enzymes, inulinases, levanases, trehalases, pectinases, and pentose sugar utilizing enzymes. In another embodiment, the enzyme can be a protease. In the context of the present disclosure, the term “protease” refers to an enzyme involved in protein digestion, metabolism and/or hydrolysis. In yet another embodiment, the enzyme can be an esterase. In the context of the present disclosure, the term “esterase” refers to an enzyme involved in the hydrolysis of an ester from an acid or an alcohol, including phosphatases such as phytases. In the fermentation process described herein, it is possible to add an exogenous source (e.g., to dose) of an enzyme to facilitate improve fermentation yield. As such, the fermentation process can comprise including one or more dose(s) of one or more enzyme(s) during the fermentation step. The exogenous enzyme that can be used during the fermentation process can include, without limitation, an alpha-amylase (such as the enzyme combination described herein), a glucoamylase, a protease, a phytase, a pullulanase, a cellulase, a hemi-cellulase such as a xylanase, a trehalase, a protease, or any combination thereof. The enzyme can be substantially purified and/or provided as part of a cocktail. The fermentation process of the present disclosure can include a step of adding a single dose (or multiple doses) of an exogenous enzyme (which may be substantially purified) to increase the fermentation yield or allow the yeast to complete the fermentation. In such embodiment, the requirement to add one or more dose(s) can be determined prior to or during fermentation.

In some embodiments, the process can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, or at least about 500 mg per hour per liter.

The present invention will be more readily understood by referring to the following examples which are given to illustrate the invention rather than to limit its scope.

### Example I—Combination of Archaeal and Bacterial Alpha-Amylases

Bacterial alpha-amylase preparations. The bacterial alpha-amylase preparation is a commercially available source of an alpha-amylase from Geobacillus stearothermophilus. Archaeal alpha-amylase preparations. Saccharomyces cerevisiae strain M27733 was engineered to express a first tethered archaeal alpha-amylase from Thermococcus hydrothermalis (pre-protein: 1-597 of SEQ ID NO: 25; mature protein: 20-597 of SEQ ID NO: 25), a second tethered archaeal alpha-amylase from Pyrococcus furiosus (pre-protein 1-486 of SEQ ID NO: 26; mature protein 20-486 of SEQ ID NO: 26) as well as additional intracellular archaeal alpha-amylases from Thermococcus hydrothermalis (pre-protein: 1-435 of SEQ ID NO: 23; mature protein: 2-435 of SEQ ID NO: 23, and pre-protein: SEQ ID NO: 30; mature protein: SEQ ID NO: 67). Strain M27733 was submitted to a fed-batch propagation and then submitted to a high pressure homogenizer.

Corn liquefaction. Corn liquefactions were conducted on 30-33% total solids corn slurry for 3 hours. A 300 g corn slurry (pH 5.2) was dosed with each respective enzyme preparation and immediately placed in a 85° C. water bath with 200 rpm mixing for the entire 3 h. The bacterial alpha-amylase preparation was dosed at 0.005% weight of enzyme per weight of dry corn. The archaeal alpha-amylase yeast product was dosed, in the absence of the bacterial alpha-amylase, at 0.03% of dry cell weight (DCW) per weight of dry corn, with the combination of the enzymes dosed at 0.005% w/w bacterial and 0.01% dcw/w archaeal. The molecular weight distribution of the resulting dextrins was obtained by size exclusion chromatography. The viscosity was measured in a 35 g liquefaction at 85° C. using the same corn slurry as the 300 g liquefactions, using the Perkin-Elmer Rapid Visco-Analyzer™. The dextrose equivalents analysis was performed using a dinitrosalicylic acid (DNS) reducing sugar assay and compared to a glucose standard.

Fermentations. Fermentations were performed at 32.5% solids from a 33% solids lab-scale liquefaction conducted at 300 g scale, pH 5.2, and 85° C. for 3 h (with the enzymes preparations described above). Each fermentation was conducted at 25 g in 250 ml Pyrex™ bottles, in triplicates, dosed with 0.3 AGU/gTs glucoamylase with 400 ppm urea. Fermentations were held at 33.3° C. for 24 h and lowered to 31.1° C. for the remaining 65 h. Final samples were collected and ethanol measured via HPLC. The mashes were fermented with Saccharomyces cerevisiae strain M12156 which has been genetically engineered to express a heterologous glucoamylase from Saccharomycopsis fibuligera and includes additional “glycerol reduction background modifications” refers the genetic modifications described in U.S. Pat. Nos. 9,605,269, 8,956,851 and US Patent Application 2022/0002661 (all incorporated in their entirety) allowing the reduction of production of glycerol.

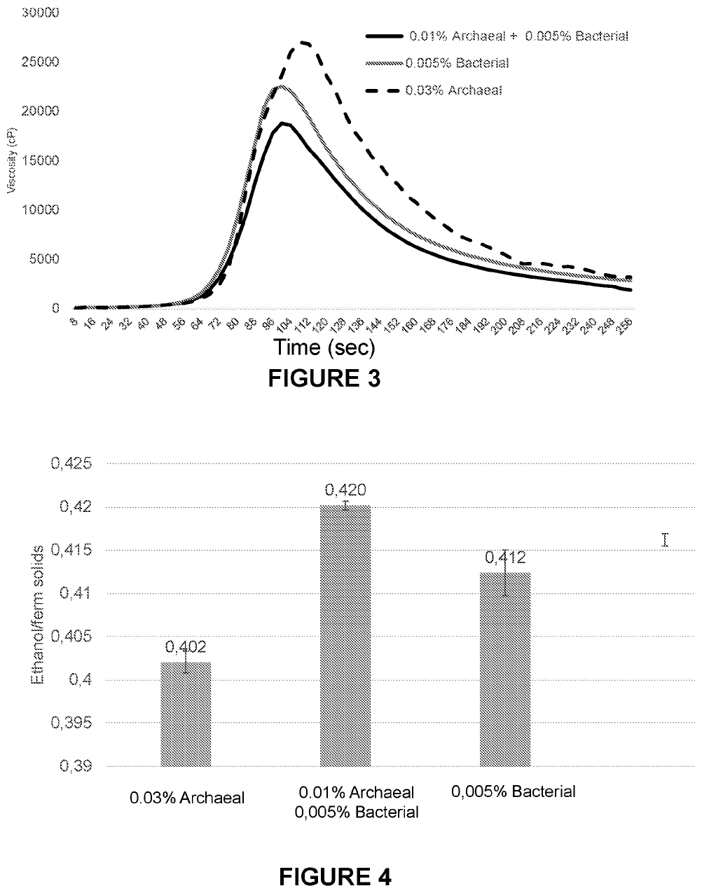

FIG. 1 depicts the oligomer profile using size exclusion chromatography of 33% corn mashes liquefied with either a commercial bacterial amylase product or an archaeal product. As shown on FIGS. 2 and 3, the combination of the archaeal and bacterial preparations improved the percentage of dextrose equivalent and viscosity profiles (with lower peaks and faster break times), when compared to the liquefaction conducted with the archaeal preparation only or with the bacterial preparation only. As shown on FIG. 4, the liquefied mash obtained with a combination of the archaeal and bacterial preparations achieved, after fermentation, a higher ethanol yield/fermentation solids, when compared to the fermentations conducted with liquefied mash obtained with the archaeal preparation only or with the bacterial preparation only.

### Example II—Combination of a Bacterial Alpha-Amylase with Multiple Archaeal Alpha-Amylases

Bacterial alpha-amylase preparations. The bacterial alpha-amylase preparation is a commercially available source of an alpha-amylase from Geobacillus stearothermophilus.

Archaeal alpha-amylase preparations. Saccharomyces cerevisiae strain M26998 was engineered to express a first tethered archaeal alpha-amylase from Thermococcus hydrothermalis (pre-protein: 1-597 of SEQ ID NO: 25; mature protein: 20-597 of SEQ ID NO: 25), a second tethered archaeal alpha-amylase from Pyrococcus furiosus (pre-protein 1-486 of SEQ ID NO: 26; mature protein 20-486 of SEQ ID NO: 26) as well as additional intracellular archaeal alpha-amylases from Thermococcus hydrothermalis (pre-protein: 1-435 of SEQ ID NO: 23; mature protein: 2-435 of SEQ ID NO: 23, and pre-protein: SEQ ID NO: 30; mature protein: SEQ ID NO: 67). Strain M26998 was submitted to a fed-batch propagation and then submitted to a high pressure homogenizer.

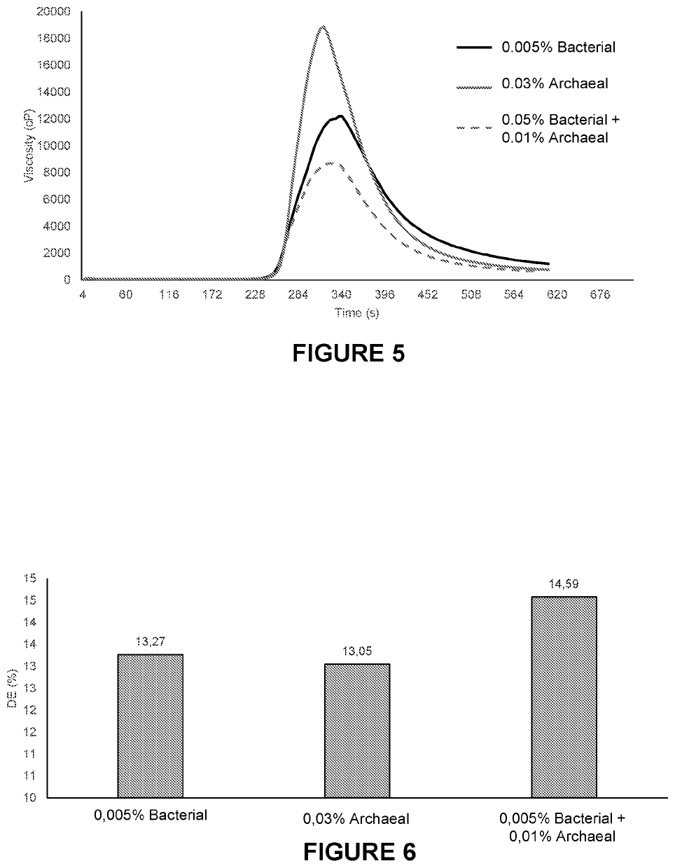

Corn liquefaction. Corn liquefactions were conducted on 30-33% total solids corn slurry for 3 hours. The temperature of a corn slurry (pH 5.2) was raised to 65° C. The enzyme preparations were then added to the liquefaction. The temperatures were ramped to 85° C. at 10° C./min and maintaining at 85° C. for the remaining time. The bacterial alpha-amylase preparation was dosed at 0.005% weight of enzyme per weight of dry corn. The archaeal alpha-amylase yeast product was dosed, in the absence of the bacterial alpha-amylase, at 0.01-0.03% of dry cell weight (DCW) per weight of dry corn. The molecular weight distribution of the resulting dextrins was obtained by size exclusion chromatography. The viscosity was measured at 85° C. using the Perkin-Elmer Rapid Visco-Analyzer™. The dextrose equivalents analysis was performed using a dinitrosalicylic acid (DNS) reducing sugar assay and compared to a glucose standard. The combination of archaeal and bacterial alpha-amylases improved both viscosity (FIG. 5) as well as dextrose equivalent percentage (FIG. 6) after liquefaction.

### Example III—Combination of Yeast-Derived Bacterial and Archaeal Alpha-Amylases

Bacterial alpha-amylase preparations. Saccharomyces cerevisiae strain M29187 was engineered to express, in an intracellular form, an alpha-amylase from Geobacillus stearothermophilus (SEQ ID NO: 28). The strain was grown in 500 mL YP-sucrose (1% yeast extract. 2% peptone, 2% sucrose) using shake flasks at 32° C. for 48 h. Cells were pelleted and the supernatant removed. The cell pellet was washed once with water before being resuspended in water to generate a yeast slurry at 11.94% dry cell weight (DCW). The cells were then disrupted using bead beating with glass beads to provide a slurry.

Archaeal alpha-amylase preparations. Saccharomyces cerevisiae strain M26684 was engineered to express, in an intracellular form, an alpha-amylase from Thermococcus hydrothermalis (SEQ ID NO: 30). The strain was grown in 500 mL YP-sucrose (1% yeast extract. 2% peptone, 2% sucrose) using shake flasks at 32° C. for 48 h. Cells were pelleted and the supernatant removed. The cell pellet was washed once with water before being resuspended in water to generate a yeast slurry at 10.66% dry cell weight (DCW). The cells were then disrupted using bead beating with glass beads to provide a slurry.

Corn liquefaction. Each respective strain was independently dosed into duplicate liquefactions. as well as a blend of each strain at 75%/25%, 50%/50%, and 25%/75% (based on DCW), with each ratio totaling 0.016 g DCW dosing. The molecular weight distribution of the resulting dextrins was obtained by size exclusion chromatography. Liquefactions were characterized on a Rapid-Visco Analyzer™ at 35 g to measure viscosity reduction for the first 5 mins and then transferred to a shaking water bath for the remaining 3 h cook time, with the temperature held at 85° C. throughout at pH 5.2. The dextrose equivalents analysis was performed using a dinitrosalicylic acid (DNS) reducing sugar assay and compared to a glucose standard. Each liquefaction was stopped by freezing the entire mash at −20° C. until fermentations were prepared.

Corn fermentation. Fermentations were performed at 34% solids at 12 g total mass in 25 mL vials, in duplicate per each duplicate liquefaction to generate quadruplicate fermentations per condition. The fermentations were dosed with 0.69 AGU/gTs of a commercial glucoamylase preparation, 500 ppm urea, and fermented using 0.05 g DCW/L of a non-genetically modified Saccharomyces cerevisiae yeast. Fermentations were held at 33.3° C. for 24 h and lowered to 31.1° C. for the remaining 65 h. Final samples were collected and ethanol measured via HPLC.

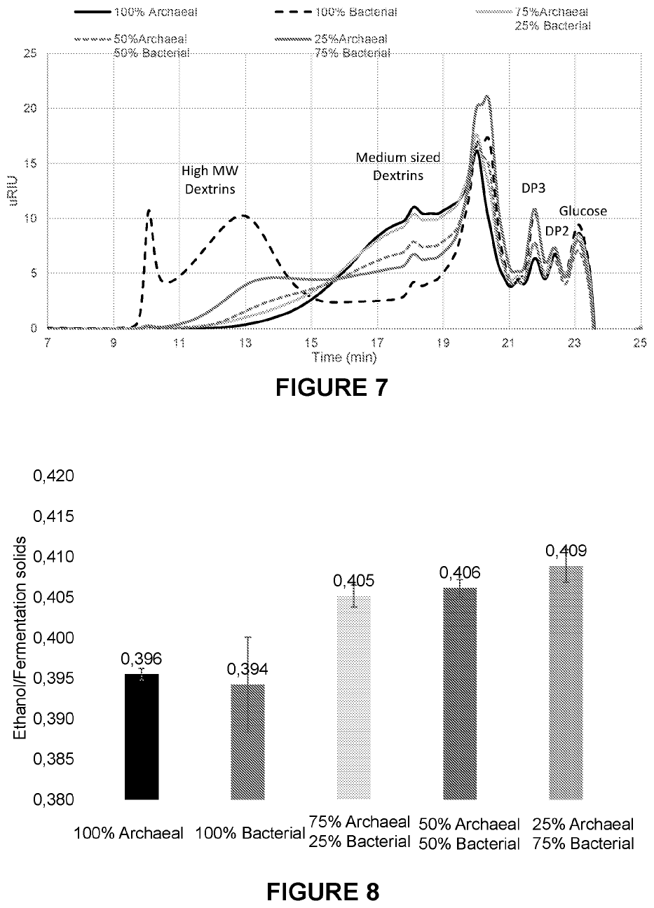

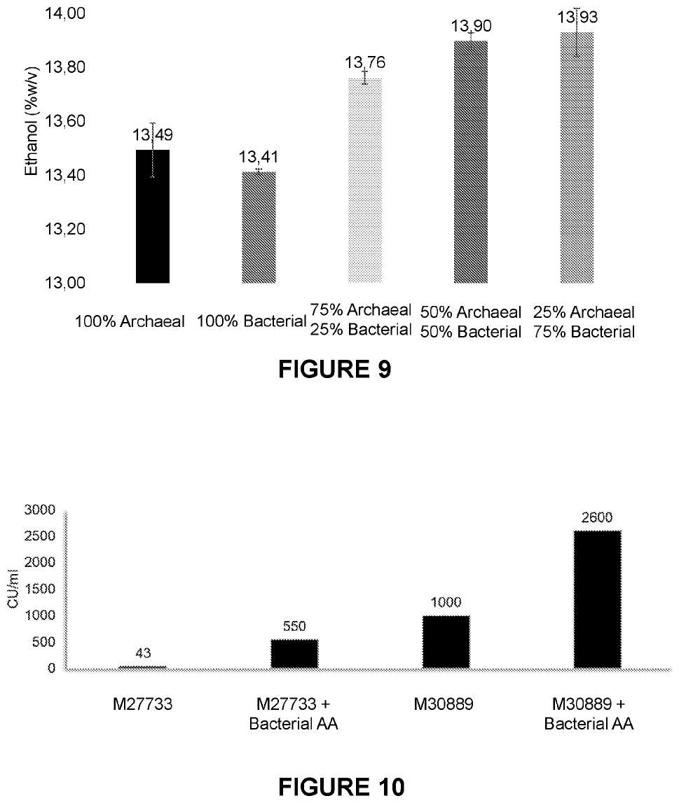

Mashes that were liquefied with bacterial alpha-amylases included higher amounts of high molecular weight dextrins, whereas mashes that were liquefied with archaeal bacterial alpha-amylases (only or in combination with bacterial alpha-amylases) had higher amounts of medium-sized molecular weight dextrins (FIG. 7). In addition, the mashes obtained with the combinations of an archaeal with a bacteria alpha-amylase provided improved ethanol yields when compared to mashes liquefied with each independent alpha-amylase. The yield benefits are observed with both the ethanol normalized to the fermentation solids (FIG. 8) or the overall ethanol (FIG. 9).

### Example IV—Yeast-Enzyme Blends

Bacterial alpha-amylase preparations. The bacterial alpha-amylase preparation is a commercially available source of an alpha-amylase from Geobacillus stearothermophilus.

Archaeal alpha-amylase preparations. Saccharomyces cerevisiae strain M27733 was engineered to express a first tethered archaeal alpha-amylase from Thermococcus hydrothermalis (pre-protein: 1-597 of SEQ ID NO: 25; mature protein: 20-597 of SEQ ID NO: 25), a second tethered archaeal alpha-amylase from Pyrococcus furiosus (pre-protein 1-486 of SEQ ID NO: 26; mature protein 20-486 of SEQ ID NO: 26) as well as additional intracellular archaeal alpha-amylases from Thermococcus hydrothermalis (pre-protein: 1-435 of SEQ ID NO: 23; mature protein: 2-435 of SEQ ID NO: 23, and pre-protein: SEQ ID NO: 30; mature protein: SEQ ID NO: 67). Strain M27733 was submitted to a fed-batch propagation and then submitted to a high pressure homogenizer (HPH).

Combined bacterial alpha-amylase preparations. Saccharomyces cerevisiae strain M30889 was engineered to co-express (i) a bacterial alpha-amylase (pre-protein: SEQ ID NO: 52; mature: SEQ ID NO: 53) in the nucleus and (ii) an archaeal alpha-amylase (pre-protein: SEQ ID NO: 54; mature: SEQ ID NO: 55) as a tethered enzyme. Strain M30889 was submitted to a fed-batch propagation and then submitted to a high pressure homogenizer.

Yeast-enzyme blends were prepared by combining yeast (M27733 or M30889) and the commercial bacterial alpha-amylase at dosages of 0.01% w/w (gDCW yeast/gDS corn) and 0.005% w/w (g AA/gDS corn), respectively. Alpha-amylase activity was measured using the Megazyme Ceralpha Assay kit (Megazyme K-CERA). Ceralpha activity is expressed as Ceralpha Units (CU) per mL of enzyme sample.

The results of the different yeast-enzyme blends are provided in FIG. 10. Under the conditions tested, the HPH preparation obtained with strain M30889 (co-expressing the archaeal and bacterial alpha-amylases) had greater alpha-amylase activity than the HPH preparation obtained with strain M27733 (expressing only archaeal alpha-amylases) alone or combined with a commercial bacterial alpha-amylase preparation.

### Example V—Bacterial Alpha-Amylases Variants

It was decided to engineer more thermostable alpha-amylases from the wild-type Geobacillus stearothermophilus alpha-amylases. G. stearothermophilus can express more than one alpha-amylases and it was decided to investigate whether the thermostability/resistance to chelating agents of these enzymes could be increased. Table 1 provides the genotypes of the Saccharomyces cerevisiae strains or isolates used in the Example.

TABLE 1
Genotypes of the Saccharomyces cerevisiae strains or isolates used in Example
V. The genetically modified strains were all derived from parental strain M10580.

| | Description of alpha- | |
|---|---|---|
| | amylase expressed | SEQ ID NO of alpha-amylase |
| Strain or isolate | (localization) | expressed |
| M10580 | None - this is a wild-type strain | |
| M25694 | Wild-type | Pre-protein SEQ ID NO: 30 |
| | (nucleus) | Mature protein SEQ ID NO: 67 |
| M27902 | Wild-type 1.0 | Pre-protein SEQ ID NO: 61 |
| | (secreted) | Mature protein SEQ ID NO: 4 |
| M27908 | Wild-type 3.0 | Pre-protein SEQ ID NO 62 |
| | (secreted) | Mature protein SEQ ID NO: 39 |
| M31186 | 1.0 ΔI181 and ΔG182 | Pre-protein SEQ ID NO: 63 |
| | (secreted) | Mature protein SEQ ID NO: 43 |
| M31187 | 3.0 ΔI181 and ΔG182 | Pre-protein SEQ ID NO: 64 |
| | (secreted) | Mature protein SEQ ID NO: 44 |
| M34404 | 3.0 ΔI181 and ΔG182 | Pre-protein SEQ ID NO: 71 |
| | (cytoplasmic) | Mature protein SEQ ID NO: 44 |
| M34413 | 3.0 ΔI181 and ΔG182 | SEQ ID NO: 45 |
| | N193F | |
| | (cytoplasmic) | |
| M34405 | 3.0 ΔI181 and ΔG182 | SEQ ID NO: 46 |
| | N193F, S242A | |
| | (cytoplasmic) | |
| M34409 | 3.0 ΔI181 and ΔG182 | SEQ ID NO: 47 |
| | N193F, S242A, P245R | |
| | (cytoplasmic) | |
| M34417 | 3.0 ΔI181 and ΔG182 | SEQ ID NO: 48 |
| | N193F, R157Y, A184T | |
| | (cytoplasmic) | |
| M34422 | 3.0 ΔI181 and ΔG182 | SEQ ID NO: 49 |
| | N193F, R157Y, A184T, | |
| | S173K | |
| | (cytoplasmic) | |
| M34426 | 3.0 ΔI181 and ΔG182 | SEQ ID NO: 50 |
| | N193F, R157Y, A184T, | |
| | T191P | |
| | (cytoplasmic) | |
| M34430 | 3.0 ΔI181 and ΔG182 | SEQ ID NO: 51 |
| | N193F, R157Y, A184T, | |
| | D281N | |
| | (cytoplasmic) | |

Thermostability assessment on gelatinized starch. S. cerevisiae strains were grown for 24-40 h. at 30° C. with shaking in YPD40 media. 10 μL of whole cell culture lysate (for intracellular strains) or supernatant (for secreted strains) was added to 50 ul of 1% (w/v) gelatinized starch in 50 mM NaOAc buffer pH 5.0. In some samples, 1, 2 or 5 mM of EGTA and/or 1 mM of CaCl₂) was added to the mixture to determine the effect of such chelating agent on the alpha-amylase activity of the enzymes being tested. In some cases, the whole cell culture lysate or supernatant was pre-incubated at an elevated temperature (as indicated in the figure legend) prior to adding substrate to determine the thermostability of the enzyme. Sample mixtures were incubated at 85° C. for 2-20 min. Starch activity was then determined by measuring the formation of reducing sugar as follows: 100 ul of 1% dinitrosalicylic acid (DNS) was added and the mixture was incubated at 99° C. for 5 min. Insoluble impurities were removed via centrifugation, and the absorbance of clarified samples was measured at 540 nm.

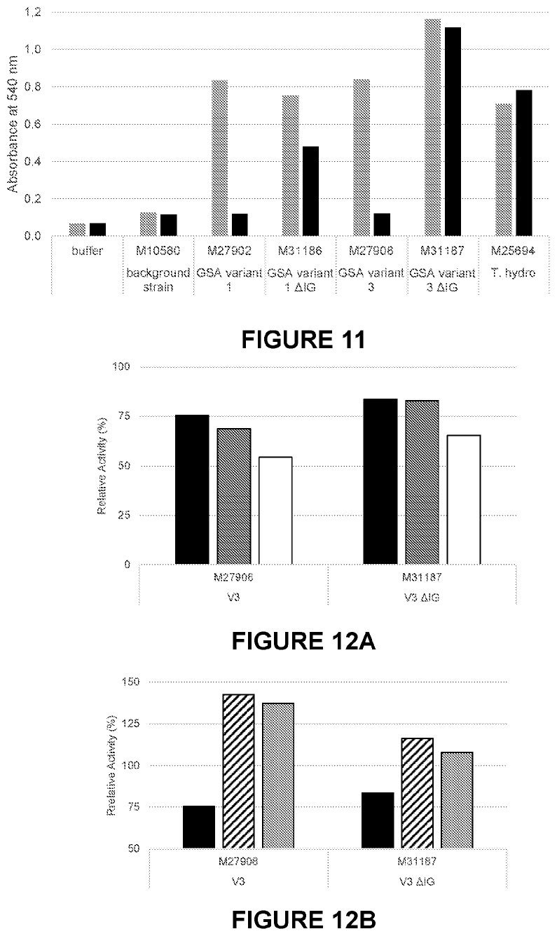

It was first determined if the deletion of I¹⁸¹ and G¹⁸² could increase the thermostability of the wild-type alpha-amylases. Strain M27902 expressed a wild-type G. stereothermophilus alpha-amylase, while strain M31186 expressed a variant of this enzyme in which I¹⁸¹ and G¹⁸² have been deleted. Strain M27908 expressed a wild-type G. stereothermophilus alpha-amylase, while strain M31187 expressed a variant of this enzyme in which I¹⁸¹ and G¹⁸² have been deleted. As shown on FIG. 11, the ΔI181/ΔG182 variant alpha-amylases (expressed by strains M31186 and M31187) have increased thermostability at 85° C., when compared to the wild-type bacterial alpha-amylases (expressed by strains M27902 and M27908 respectively). The thermostability of the enzyme expressed by strain M31187 exhibited higher thermostability than the archaeal alpha-amylase expressed by strain M25694.

It was also determined if the presence of a chelating agent and/or calcium could have an impact on the alpha-amylase activity of the various enzymes. The alpha-amylases expressed by S. cerevisiae strains M27908 and M31187 were assayed for gelatinized starch activity as indicated above, in the presence and in the absence of 1, 2 or 5 mM EGTA, optionally in the presence of 1 mM of CaCl₂). Relative activity was expressed as a percentage of activity with or without the addition of 1 mM EGTA. As shown on FIG. 12A, the variant alpha-amylase expressed by strain M31187 bound the calcium ion more tightly, thus making it less susceptible to chelating agents, such as EGTA. To confirm that the decrease in relative activity in the presence of EGTA was directly related to calcium chelation and not a result of EGTA toxicity or other inhibitory effects, 1 mM EGTA was supplemented with 1 mM CaCl₂). As shown on FIG. 12B, not only did the CaCl₂) addition restore activity in the presence of EGTA, but relative activity surpassed 100% for all enzymes tested. A similar trend was seen with the addition of 1 mM CaCl₂) alone, confirming that the calcium is directly influencing alpha-amylase activity. More importantly, the enzyme expressed by strain M31187 was more resilient to added calcium, suggesting that the variant alpha-amylase functioned more independently from exogenous doses of calcium and chelating agents.

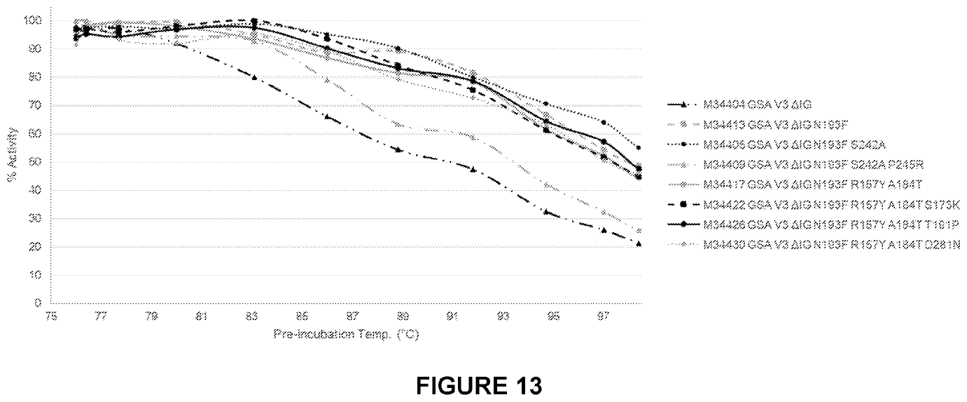

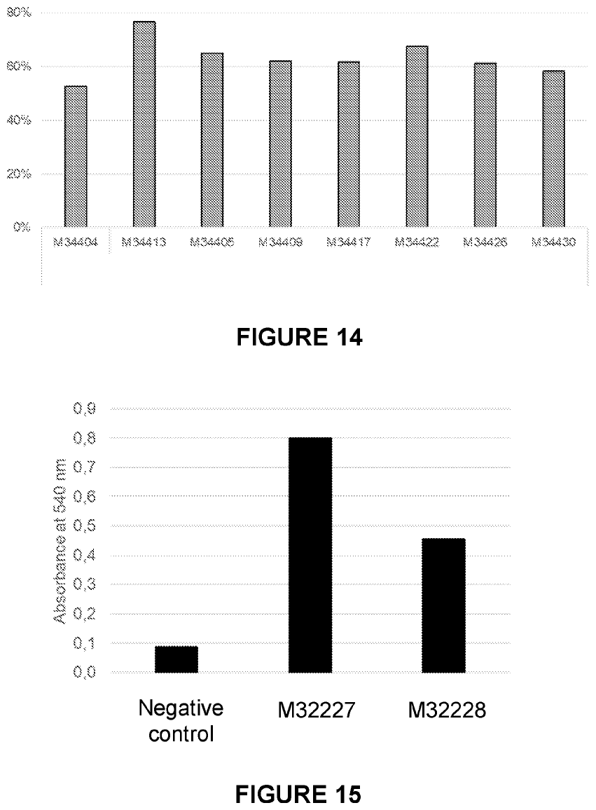

It was then determined if the thermostability/resistance to chelating agents of the variant alpha-amylases comprising the deletion of I¹⁸¹ and G¹⁸² residues could further be increased. To this end, various additional variants comprising one or more amino acid substitutions (listed in Table 1) were tested. As shown on FIG. 13, the additional variants were all more thermostable than the initial 3.0 ΔI181/ΔG182 variant. As also shown on FIG. 14, the additional variants were all more resistant to EGTA than the initial 3.0 ΔI181/ΔG182 variant.

### Example VI—Archaeal Alpha-Amylases Variants

TABLE 2
Genotypes of the Saccharomyces cerevisiae strains used in
Example VI. The genetically modified strains were all derived
from parental strain M10580 (described in Example V).

| | Description of alpha- | |
|---|---|---|
| | amylase expressed | SEQ ID NO of alpha-amylase |
| Strain or isolate | (localization) | expressed |
| M25694 | Wild-type | Pre-protein SEQ ID NO: 30 |
| | (nucleus) | Mature protein SEQ ID NO: 67 |
| M30813 | C385Q, C429V | Pre-protein: SEQ ID NO: 56 |
| | (nucleus) | Mature protein : SEQ ID NO: 57 |
| M30818 | Y123N, C385Q, C429V | Pre-protein: SEQ ID NO: 70 |
| | (nucleus) | Mature protein: SEQ ID NO: 59 |

Thermostability assessment on gelatinized starch. S. cerevisiae strains were grown for 48 h. at 30° C. with shaking in YP media with 20 g/L sucrose. Ten (10) μL of whole cell lysate (obtained by bead beating) or supernatant was pre-incubated at elevated temperatures of 75-100° C. for 10 min. Following this pre-incubation step, 50 ul of 1% (w/v) gelatinized starch in 50 mM NaOAc buffer pH 5.0 was added and samples were incubated at 85° C. for 5 min. Starch activity was then determined by measuring the formation of reducing sugar as follows: 100 ul of 1% dinitrosalicylic acid (DNS) was added and the mixture was incubated at 99° C. for 5 min. Insoluble impurities were removed via centrifugation, and the absorbance of clarified samples was measured at 540 nm.

Phenotypic observations. Visual inspection (in liquid medium and on solid medium) as well as microscopic analysis were conducted to determine the phenotypes of the different yeast strains.

It was noticed that S. cerevisiae strains expressing a wild-type archaeal alpha-amylase (M25694) had a rugose phenotype (when grown on solid medium) and a tendency to flocculate (when grown in liquid medium, see Table 3). It was decided to engineer a S. cerevisiae strain expressing alpha-amylases which would limit flocculation and the rugose phenotype, as these features can be usually considered detrimental during commercial propagation. Two substitutions at cysteines 385 and 429 were introduced in the wild-type alpha amylase and this variant enzyme was expressed in strain M30813. As shown in Table 3, strain M30813 had less of a tendency to flocculate (when compared to strain M25694) but still remained rugose. A further substitution was introduced at position 123 and this variant enzyme was expressed in strain M30818. As shown in Table 3, strain M30818 did not flocculate and exhibited a smooth phenotype when grown on a solid medium.

TABLE 3
Phenotypic analysis of S. cerevisiae strains M25694, M30813, and M30818.

| | | Tendency to | | | |
|---|---|---|---|---|---|
| | Tendency | form | | Alpha-amylase | |
| | to | aggregates | | activity and | % in |
| | flocculate | determined | Smooth or | thermostability | intracellular |
| | in liquid | by light | rugose | on gelatinized | enzyme |
| Strain | medium | microscopy | phenotype | starch | retention |
| M25694 | +++ | +++ | Rugose | +++ | 46 |
| M30813 | + | + | Rugose | +++ | 68 |
| M30818 | − | − | Smooth | +++ | 88 |

The hydrolysis activity and thermostability of the alpha-amylase enzymes were compared between the strain expressing the wild-type archaeal alpha-amylase and the two variants presented in Table 2. As indicated in Table 3, the substitutions introduced at positions 123, 385, and 429 do not negatively affect hydrolysis activity or thermostability of the enzyme. In addition, the alpha-amylase expressed in strains M30813 and M30818 exhibited an increase in the percentage of the intracellular alpha-amylase activity when compared to the alpha-amylase expressed in strain M25694, further suggesting that the strains expressing the variant enzymes are more stable.

### Example VII—Expression of Alpha-Amylases in Komagataella and Bacillus Host Cells

TABLE 4
Genotypes of the Saccharomyces cerevisiae, Komagataella phaffii,
and Bacillus subtilis strains used in Example VII.

| | | Description of alpha- | |
|---|---|---|---|
| | | amylase expressed | SEQ ID NO of alpha-amylase |
| Designation | Host cell | (localization) | expressed |
| M32227 | K. phaffi | G. stereothermophilus | Pre-protein SEQ ID NO: 64 |
| | | 3.0 ΔI181 and ΔG182 | Mature protein SEQ ID NO: 44 |
| | | (secreted) | |
| M32228 | K. phaffi | T. hydrothermalis Y123N, | Pre-protein: SEQ ID NO: 58 |
| | | C385Q, C429V | Mature protein: SEQ ID NO: 59 |
| | | (secreted) | |
| M2390 | S. cerevisiae | None - these are wild-type strains | |
| M10580 | | | |
| M20672 | S. cerevisiae | T. hydrothermalis | SEQ ID NO: 23 |
| | | (cytoplasmic) | |
| | | P. furiosus | Pre-protein: SEQ ID NO: 66 |
| | | (secreted) | Mature protein: SEQ ID NO: 14 |
| M27902 | S. cerevisiae | G. stereothermophilis 1.0 | Pre-protein SEQ ID NO: 61 |
| | | (secreted) | Mature protein SEQ ID NO: 4 |
| M27908 | S. cerevisiae | G. stereothermophilis 3.0 | Pre-protein SEQ ID NO 62 |
| | | (secreted) | Mature protein SEQ ID NO: 39 |
| M21018 | B. subtilis | None - this is a wild-type strain | |
| M22569 | B. subtilis | T. hydrothermalis wild- | Pre-protein SEQ ID NO: 19 |
| | | type | Mature protein: SEQ ID NO: 13 |
| | | (cytoplasmic) | |
| M27597 | B. subtilis | G. stereothermophilis 1.0 | Pre-protein: SEQ ID NO: 69 |
| | | (secreted) | Mature protein: SEQ ID NO: 4 |
| M27599 | B. subtilis | G. stereothermophilis 3.0 | Pre-protein: SEQ ID NO: 68 |
| | | (secreted) | Mature protein: SEQ ID NO: 39 |

Thermostability assessment on gelatinized starch. S. cerevisiae strains were grown for 24-40 h. at 30° C. with shaking in YPD40 media. B. subtilis strains were grown for 24 h. at 37° C. with shaking in LB or LB/Kan5 media. K. phaffi strains were grown for 24 h. at 28° C. with shaking in YP media with 20 g/L sucrose. K. phaffi strains were then submitted to a 24 h. batch propagation in Fermentation BsasI salts medium (26.7 g/L phosphoric acid, 0.93 g/L calcium sulfate, 18.2 g/L potassium sulfate, 14.9 g/L magnesium sulfate×7H₂O, 4.13 g/L potassium hydroxide, and 40 g/L glycerol), followed by a 48 h. fed-batch propagation with a MeOH/glycerol feed. Ten (10) μL of whole cell culture lysate (for intracellular strains) or supernatant (for secreted strains) was added to 50 μL of 1% (w/v) gelatinized starch in 50 mM NaOAc buffer pH 5.0. In some cases, the whole cell culture lysate or supernatant was pre-incubated at 85° C. prior to adding substrate (as indicated in the figure legend). Sample mixtures were incubated at 85° C. for 10-30 min. Starch activity was then determined by measuring the formation of reducing sugar as follows: 100 ul of 1% dinitrosalicylic acid (DNS) was added and the mixture was incubated at 99° C. for 5 min. Insoluble impurities were removed via centrifugation, and the absorbance of clarified samples was measured at 540 nm.

A bacterial and an archaeal alpha-amylase were expressed independently in two different K. phaffii strains M32227, and M32228 (see Table 4). As shown on FIG. 15, both of these alpha-amylases exhibited alpha-amylase activity on gelatinized starch.

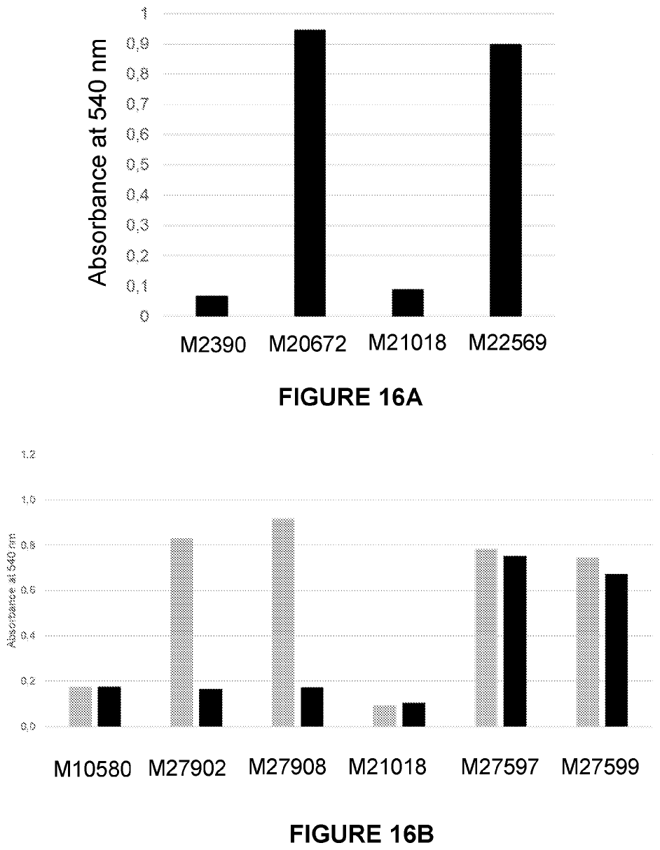

A bacterial and an archaeal alpha-amylase were expressed independently in different S. cerevisiae (M20672, M27902, and M27908) and B. subtilis (M22569, M27597, and M27599) strains (see Table 3). As shown on FIGS. 16A and 16B, all of these alpha-amylases expressed in S. cerevisiae (M20672, M27902, and M27908) and B. subtilis (M22569, M27597, and M27599) exhibited alpha-amylase activity on gelatinized starch.

While the invention has been described in connection with specific embodiments thereof, it will be understood that the scope of the claims should not be limited by the preferred embodiments set forth in the examples, but should be given the broadest interpretation consistent with the description as a whole.

1. An enzyme combination comprising at least one archaeal alpha-amylase and at least one bacterial alpha-amylase.

2.-24. (canceled)

25. A recombinant microbial host cell capable of expressing the enzyme combination defined in claim 1.

26.-30. (canceled)

31. An inactivated microbial product comprising the enzyme combination defined in claim 1 and a component of a recombinant microbial host cell having expressed the enzyme combination.

32. A population of recombinant microbial host cells comprising a first subpopulation of recombinant microbial host cells capable of expressing at least one archaeal alpha-amylase and a second subpopulation of recombinant microbial host cells capable of expressing at least one bacterial alpha-amylase.

33.-37. (canceled)

38. An inactivated microbial product comprising (i) the enzyme combination defined in claim 1 and (ii) a component of a first subpopulation of recombinant microbial host cells having expressed the at least one archaeal alpha-amylase and/or a second subpopulation of recombinant microbial host cells having expressed the at least one bacterial alpha-amylase.

39.-41. (canceled)

42. A process for making an hydrolyzed liquefaction medium, the process comprising (i) contacting an untreated liquefaction medium with at least one archaeal alpha-amylase and at least one bacterial alpha-amylase and (ii) hydrolyzing the untreated liquefaction medium to generate the hydrolyzed liquefaction medium.

43.-49. (canceled)

50. A process for making a fermented product, the process comprising contacting the hydrolyzed liquefaction medium, obtainable by the process of claim 42 with a fermenting yeast under a condition to allow the conversion of the hydrolyzed liquefaction medium into a fermentation product.

51.-54. (canceled)

55. A variant polypeptide having alpha-amylase activity, wherein the variant polypeptide has at least 70% identity and less than 100% identity to the amino acid sequence of SEQ ID NO: 13, and is less dependent on the presence of a metallic ion, more thermostable and/or more resistant to chelation than the polypeptide consisting of the amino acid sequence of SEQ ID NO: 13.

56. The variant polypeptide of claim 55 having one or more amino acid residue substitutions.

57.-62. (canceled)

63. A recombinant microbial host cell capable of expressing the variant polypeptide of claim 55.

64. An inactivated microbial product comprising the variant polypeptide of claim 55 and a component of a recombinant microbial host cell having expressed the variant polypeptide.

65. (canceled)

66. A process for making an hydrolyzed liquefaction medium, the process comprising (i) contacting an untreated liquefaction medium with the variant polypeptide of claim 55 and (ii) hydrolyzing the untreated liquefaction medium to generate the hydrolyzed liquefaction medium.

67. A process for making a fermented product, the process comprising contacting the hydrolyzed liquefaction medium obtainable by the process of claim 66 with a fermenting yeast under a condition to allow the conversion of the hydrolyzed liquefaction medium into a fermentation product.

68. A variant polypeptide having alpha-amylase activity, wherein the variant polypeptide has at least 70% identity and less than 100% identity with the amino acid sequence of SEQ ID NO: 39, and is less dependent on a metallic ion, more thermostable and/or more resistant to chelation than the polypeptide consisting of the amino acid sequence of SEQ ID NO: 39.

69. The variant polypeptide of claim 68 having one or more amino acid residue deletion.

70.-73. (canceled)

74. A recombinant microbial host cell capable of expressing the variant polypeptide of claim 68.

75. An inactivated microbial product comprising the variant polypeptide of claim 68 and a component of a recombinant microbial host cell having expressed the variant polypeptide.

76. (canceled)

77. A process for making an hydrolyzed liquefaction medium, the process comprising (i) contacting an untreated liquefaction medium with the variant polypeptide of claim 68 and (ii) hydrolyzing the untreated liquefaction medium to generate the hydrolyzed liquefaction medium.

78. A process for making a fermented product, the process comprising contacting the hydrolyzed liquefaction medium obtainable by the process of claim 77 with a fermenting yeast under a condition to allow the conversion of the hydrolyzed liquefaction medium into a fermentation product.
